SEQUENCE LISTING (40 sequences)

SEQ ID NO: 1 - protein, 379 aa, Artificial Sequence
  Polypeptide sequence of Amatilin
MKYLLPTAAAGLLLLAAQPAMAMGRICRCICGRGICRCICGVPGVGVPGVGGATGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWALWKTMLKELGTMALHAGKAALGAAADTISQGTQVPGVGVPGVGKLAAALEHHHHHH

SEQ ID NO: 2 - DNA, 1140 bp, Artificial Sequence
  Coding sequence of Amatilin
atgaaatacctgctgccgaccgctgctgctggtctgctgctcctcgctgcccagccggcgatggccatggggcgtatttgccgttgcatttgcggccgtggcatttgccgctgcatctgtggcgtgccgggtgttggtgttccgggtgtgggtggtgcgaccggatccgatgtgaactttgatctgagcaccgcgaccgcgaaaacctataccaaattcatcgaagattttcgtgcgaccctgccgtttagccataaagtgtatgatatcccgctgctgtatagcaccattagcgatagccgtcgttttattctgctggatctgaccagctatgcgtatgaaaccattagcgtggcgattgatgtgaccaacgtgtatgtggtggcgtatcgtacccgtgatgtgagctactttttcaaagaaagcccgccggaagcgtacaacattctgtttaaaggcacccgtaaaattaccctgccgtataccggcaactatgaaaacctgcagaccgcggcgcataaaattcgtgaaaacatcgatctgggcctgccggccctgagcagcgcgattaccaccctgttttattataacgcgcagagcgcgccgagcgcgctgctggtgctgattcagaccaccgcggaagcggcgcgttttaaatatattgaacgccacgtggcgaaatatgtggcgaccaactttaaaccgaacctggccattattagcctggaaaaccagtggagcgccctgagcaaacaaatttttctggcccagaaccagggcggcaaatttcgtaatccggtggatctgattaaaccgaccggcgaacgttttcaggtgaccaatgtggatagcgatgtggtgaaaggcaacattaaactgctgctgaacagccgtgcgagcaccgcggatgaaaactttattaccaccatgaccctgctgggcgaaagcgtggtggaattcccgtgggcgctgtggaaaaccatgctgaaagaactgggcaccatggcgctgcatgcgggtaaagcggcgctgggtgcggcagcggataccattagccagggcacccaggttccgggcgtgggcgttccgggcgttggtaagcttgcggccgcactcgagcaccaccaccaccaccactga

SEQ ID NO: 3 - protein, 5 aa, Artificial Sequence
  A polypeptide of the present invention
VPXVG

SEQ ID NO: 4 - protein, 18 aa, Artificial Sequence
  A polypeptide of the present invention
GRICRCICGRGICRCICG

SEQ ID NO: 5 - protein, 268 aa, Artificial Sequence
  A polypeptide of the present invention
GSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 6 - protein, 34 aa, Artificial Sequence
  A polypeptide of the present invention
ALWKTMLKELGTMALHAGKAALGAAADTISQGTQ

SEQ ID NO: 7 - protein, 18 aa, Artificial Sequence
  A polypeptide sequence of naturally occurring and synthetic theta Defensin proteins
GFCRCLCRRGVCRCICTR

SEQ ID NO: 8 - protein, 18 aa, Artificial Sequence
  A polypeptide sequence of naturally occurring and synthetic theta Defensin proteins
RCLCRRGVCRCLCRRGVC

SEQ ID NO: 9 - protein, 18 aa, Artificial Sequence
  A polypeptide sequence of naturally occurring and synthetic theta Defensin proteins
RCICTRGFCRCICTRGFC

SEQ ID NO: 10 - protein, 18 aa, Artificial Sequence
  A polypeptide sequence of naturally occurring and synthetic theta Defensin proteins
GICRCICGRGICRCICGR

SEQ ID NO: 11 - protein, 18 aa, Artificial Sequence
  A polypeptide sequence of naturally occurring and synthetic theta Defensin proteins
GICRCICGRGICRCICGR

SEQ ID NO: 12 - protein, 18 aa, Artificial Sequence
  A polypeptide sequence of naturally occurring and synthetic theta Defensin proteins
RICRCICGRRICRCICGR

SEQ ID NO: 13 - protein, 18 aa, Artificial Sequence
  A polypeptide sequence of naturally occurring and synthetic theta Defensin proteins
GICRCICGRGICRCICGR

SEQ ID NO: 14 - protein, 18 aa, Artificial Sequence
  A polypeptide sequence of naturally occurring and synthetic theta Defensin proteins
GICRCICGKGICRCICGR

SEQ ID NO: 15 - protein, 18 aa, Artificial Sequence
  A polypeptide sequence of naturally occurring and synthetic theta Defensin proteins
GICRCYCGRGICRCICGR

SEQ ID NO: 16 - protein, 18 aa, Artificial Sequence
  A polypeptide sequence of naturally occurring and synthetic theta Defensin proteins
GICRCICGRGICRCYCGR

SEQ ID NO: 17 - protein, 18 aa, Artificial Sequence
  A polypeptide sequence of naturally occurring and synthetic theta Defensin proteins
GYCRCICGRGICRCICGR

SEQ ID NO: 18 - protein, 18 aa, Artificial Sequence
  A polypeptide sequence of naturally occurring and synthetic theta Defensin proteins
GICRCICGRGYCRCICGR

SEQ ID NO: 19 - protein, 18 aa, Artificial Sequence
  A polypeptide sequence of naturally occurring and synthetic theta Defensin proteins
GICYCICGRGICRCICGR

SEQ ID NO: 20 - protein, 18 aa, Artificial Sequence
  A polypeptide sequence of naturally occurring and synthetic theta Defensin proteins
GICICICGYGICRCICGR

SEQ ID NO: 21 - protein, 18 aa, Artificial Sequence
  A polypeptide sequence of naturally occurring and synthetic theta Defensin proteins
GICICICGRGICYCICGR

SEQ ID NO: 22 - protein, 18 aa, Artificial Sequence
  A polypeptide sequence of naturally occurring and synthetic theta Defensin proteins
GICICICGRGICYCICGR

SEQ ID NO: 23 - protein, 18 aa, Artificial Sequence
  A polypeptide sequence of naturally occurring and synthetic theta Defensin proteins
RGCICRCIGRGCICRCIG

SEQ ID NO: 24 - protein, 18 aa, Artificial Sequence
  A polypeptide sequence of naturally occurring and synthetic theta Defensin proteins
RGCICRCIGRGCICRCIG

SEQ ID NO: 25 - protein, 18 aa, Artificial Sequence
  A polypeptide sequence of naturally occurring and synthetic theta Defensin proteins
GICRCICGRGICRCICGR

SEQ ID NO: 26 - protein, 18 aa, Artificial Sequence
  A polypeptide sequence of naturally occurring and synthetic theta Defensin proteins
GICRCICGKGICRCYCGR

SEQ ID NO: 27 - protein, 4 aa, Artificial Sequence
  An example of fusion proteins according to the present invention
GGGS

SEQ ID NO: 28 - protein, 360 aa, Artificial Sequence
  An example of fusion proteins according to the present invention
SFGLCRLRRGFCAHGRCRFPSIPIGRCSRFVQCCRRVWVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNSCASRCKGHCRARRCGYYVSVLYRGRCYCKCLRCVPGVGVPGVG

SEQ ID NO: 29 - protein, 401 aa, Artificial Sequence
  An example of fusion proteins according to the present invention
LEKRKWKLFKKIEKVGQRVRDAVISAGPAVATVAQATALAKNVPGVGVPGVGGATGSDVSFRLSGATSKKKVYFISNLRKALPNEKKLYDIPLVRSSSGSKATAYTLNLANPSASQYSSFLDQIRNNVRDTSLIYGGTDVAVIGAPSTTDKFLRLNFQGPRGTVSLGLRRENLYVVAYLAMDNANVNRAYYFKNQITSAELTALFPEVVVANQKQLEYGEDYQAIEKNAKITTGDQSRKELGLGINLLITMIDGVNKKVRVVKDEARFLLIAIQMTAEAARFRYIQNLVTKNFPNKFDSENKVIQFQVSWSKISTAIFGDCKNGVFNKDYDFGFGKVRQAKDLQMGLLKYLGRPKSSSIEANSTDDTADVLVPGVGVPGVGKTCENLADTFRGPCFATSNC

SEQ ID NO: 30 - protein, 297 aa, Artificial Sequence
  An example of fusion proteins according to the present invention
MGRICRCICGRGICRCICGVPGVGVPGVGGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 31 - protein, 297 aa, Artificial Sequence
  An example of fusion proteins according to the present invention
MGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWVPGVGVPGVGGRICRCICGRGICRCICG

SEQ ID NO: 32 - protein, 313 aa, Artificial Sequence
  An example of fusion proteins according to the present invention
MGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPWVPGVGVPGVGALWKTMLKELGTMALHAGKAALGAAADTISQGTQ

SEQ ID NO: 33 - protein, 313 aa, Artificial Sequence
  An example of fusion proteins according to the present invention
MALWKTMLKELGTMALHAGKAALGAAADTISQGTQVPGVGVPGVGGSDVNFDLSTATAKTYTKFIEDFRATLPFSHKVYDIPLLYSTISDSRRFILLDLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVEFPW

SEQ ID NO: 34 - protein, 328 aa, Artificial Sequence
  An example of fusion proteins according to the present invention
VPGVGVPGVGKWCFRVCYRGICYRRCRVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNVPGVGVPGVGHGVSGHGQHGVHG

SEQ ID NO: 35 - protein, 336 aa, Artificial Sequence
  An example of fusion proteins according to the present invention
VPGVGVPGVGFLPLLAGLAANFLPTIICFISYKCVPGVGVPGVGGATGSDVNFDLSTATAKTYTKFIEDFRATLPFSHKVYDIPLLYSTISDSRRFILLNLTSYAYETISVAIDVTNVYVVAYRTRDVSYFFKESPPEAYNILFKGTRKITLPYTGNYENLQTAAHKIRENIDLGLPALSSAITTLFYYNAQSAPSALLVLIQTTAEAARFKYIERHVAKYVATNFKPNLAIISLENQWSALSKQIFLAQNQGGKFRNPVDLIKPTGERFQVTNVDSDVVKGNIKLLLNSRASTADENFITTMTLLGESVVNVPGVGVPGVGKLAKLAKKLAKLAK

SEQ ID NO: 36 - protein, 325 aa, Artificial Sequence
  Polypeptide sequence of RetroGAD1
GICRCIGRGICRCICGRVPGVGVPGVGGATGSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPKGLWSKIKEAAKAAGKAALNAVTGLVNQGDQPSVPGVGVPGVG

SEQ ID NO: 37 - DNA, 1185 bp, Artificial Sequence
  Coding sequence of Amatilin
gggcagtgagcggaaggcccatgaggccagttaattaagaggtaccgaattctcattcggtttgtgtagattgagaagaggtttctgtgctcacggtagatgtagattcccatccatcccaatcggtagatgttccagattcgttcagtgttgtagaagagtttgggtcccaggtgttggtgttccaggtgttggaggtgctactggttctgatgttaacttcgacttgtccactgctactgctaagacttacactaagttcatcgaggacttcagagctactttgccattctcccacaaggtttacgacatccctttgttgtactccactatctccgactccagaagattcatcttgttgaacttgacttcctacgcttacgagactatctccgttgctatcgacgttacaaacgtttacgttgttgcttacagaactagagatgtttcctacttcttcaaagagtccccaccagaggcttacaacatcttgttcaagggtactagaaagatcactttgccatacactggtaactacgagaacttgcagactgctgctcacaagatcagagagaacatcgacttgggtttgccagctttgtcctccgctatcactactttgttctactacaacgctcagtccgctccatccgctttgttggttttgatccagactactgctgaggctgctagattcaagtacatcgagagacacgttgctaagtacgttgctacaaacttcaagccaaacttggctatcatctccttggagaaccagtggtctgctttgtccaagcagatcttcttggctcaaaaccagggtggtaagttcagaaacccagtcgacttgatcaagccaaccggtgagagattccaggttactaatgttgactccgacgttgttaagggtaacatcaagttgttgttgaactccagagcttccactgctgacgagaacttcatcactactatgactttgttgggtgagtccgttgttaactcctgtgcttccagatgtaagggtcactgtagagctagaagatgtggttactacgtttccgttctgtacagaggtagatgttactgtaaatgtttgagatgtgtccccggtgttggagtccctggtgtcggtgcggccgcgagctcatggcgcgcctaggccttgacggccttccgccaattcgc

SEQ ID NO: 38 - DNA, 1084 bp, Artificial Sequence
  Coding sequence for RetroGAD1
cgaattggcggaaggccgtcaaggccacgtgtcttgtccaggtaccgaattcggaatctgtagatgcatctgcggtagaggtatctgcagatgtatttgtggaagagtcccaggtgttggtgttccaggtgttggaggtgctactggttctggtttggacactgtttcattctccactaagggtgctacttacatcacttacgttaactttttgaacgagttgagagttaagttgaagccagagggtaactcccacggtatccctttgttgagaaagaagtgtgacgacccaggtaagtgtttcgttttggttgctttgtccaacgacaacggtcagttggctgagattgctatcgacgttacttccgtttacgttgttggttaccaggttagaaacagatcctacttcttcaaggacgctccagacgctgcttacgaaggtttgttcaagaacactatcaagactagattgcacttcggtggttcctacccatctttggaaggtgagaaggcttacagagagactactgacttgggtatcgagccattgagaatcggtatcaagaagttggacgagaacgctatcgacaactacaagccaactgagatcgcttcctccttgttggttgttatccagatggtttccgaggctgctagattcactttcatcgagaaccagatcagaaacaacttccagcagagaatcagaccagctaacaacactatttccttggagaacaagtggggtaagttgtccttccagatcagaacatccggtgctaacggtatgttctctgaggctgttgagttggagagagctaacggtaagaagtactacgttactgctgttgaccaggttaagccaaagatcgctttgttgaagttcgttgacaaggacccaaagggtttgtggtccaagatcaaagaggctgctaaggctgctggtaaggctgctttgaatgctgttactggtttggttaaccagggtgaccaaccatctgtccctggtgttggagtccctggtgtcggtgcggccgcgagctctggagcacaagactggcctcatgggccttccgctcactgc

SEQ ID NO: 39 - DNA, 998 bp, Artificial Sequence
  Coding sequence of Tampal 1
ggatccgttccgggtgtgggtgttccgggtgttggtaaatggtgtttcgtgtttgttatcgcggtatttgttatcgtcgttgtcgtgtgccaggcgttggcgttccaggcgtgggtggtgcaaccggtagtgatgttaattttgatctgagcaccgcaaccgcaaaaacctataccaaatttatcgaagattttcgtgcaaccctgccgtttagccataaagtttatgatattccgctgctgtatagcaccattagcgatagccgtcgttttattctgctgaatctgaccagctatgcctatgaaaccattagcgttgcaattgatgtgaccaatgtttatgttgttgcatatcgtacccgtgatgtgagctattttttcaaagaaagccctccggaagcctataacattctgtttaaaggcacccgcaaaatcaccctgccgtataccggtaattatgaaaatctgcagaccgcagcacataaaattcgcgaaaatattgatctgggtctgcctgcactgagcagcgcaattaccaccctgttttattacaatgcacagagcgcaccgagcgcactgctggttctgattcagaccaccgcagaagcagcacgctttaaatacattgaacgtcatgttgccaaatacgtggccaccaactttaaaccgaatctggcaattattagcctggaaaatcagtggtcagcactgagcaaacaaatttttctggcacagaatcagggtggcaaatttcgtaatccggttgatctgattaaaccgaccggtgaacgttttcaggttaccaatgttgatagtgatgtggtgaaaggcaacattaaactgctgctgaatagccgtgcaagcaccgcagatgaaaactttattaccaccatgaccctgctgggtgaaagcgttgttaatgttcctggtgttggcgtgcctggtgttggtcatggtgttagcggtcatggtcagcatggtgttcatggttaaaagctt

SEQ ID NO: 40 - DNA, 1023 bp, Artificial Sequence
  Coding sequence of K5
ggatccgttccgggtgtgggtgttccgggtgttggctttctgggtgcactgtttaaagttgcaagcaaagttctgccgagcgttaaatgtgcaattaccaaaaaatgtgttcctggcgttggtgttccaggcgtgggtggtgcaaccggtagtgatgttaattttgatctgagcaccgcaaccgcaaaaacctataccaaatttatcgaagattttcgtgcaaccctgccgtttagccataaagtttatgatattccgctgctgtatagcaccattagcgatagccgtcgttttattctgctgaatctgaccagctatgcctatgaaaccattagcgttgcaattgatgtgaccaatgtttatgttgttgcatatcgtacccgtgatgtgagctattttttcaaagaaagccctccggaagcctataacattctgtttaaaggcacccgcaaaatcaccctgccgtataccggtaattatgaaaatctgcagaccgcagcacataaaattcgcgaaaatattgatctgggtctgcctgcactgagcagcgcaattaccaccctgttttattacaatgcacagagcgcaccgagcgcactgctggttctgattcagaccaccgcagaagcagcacgctttaaatacattgaacgtcatgttgccaaatacgtggccaccaactttaaaccgaatctggcaattattagcctggaaaatcagtggtcagcactgagcaaacaaatttttctggcacagaatcagggtggcaaatttcgtaatccggttgatctgattaaaccgaccggtgaacgttttcaggttaccaatgttgatagtgatgtggtgaaaggcaacattaaactgctgctgaatagccgtgcaagcaccgcagatgaaaactttattaccaccatgaccctgctgggtgaaagcgttgttaatgttccaggtgttggtgtgcctggtgtgggtaaactggcaaaactggccaaaaaactggctaagctggcgaaataaaagctt

(19) United States
(12) Patent Application Publication
(10) Pub. No.: US 2015/0284438 A1
(43) Pub. Date: Oct. 8, 2015

(54) DOSAGE REGIME OF FUSION COMPOUNDS

(71) Applicant: BioValence Sdn. Bhd., Petaling, Jaya (MY)

(72) Inventors: Shamala Devi K C Sekaran, Petaling Jaya (MY); Hussin A. Rothan, Kuala Lumpur (MY); Eng Huan Ung, Kota Kinabalu (MY); Ag. Muhammad Sagaf Abu Bakar, Kota Kinabalu (MY)

(73) Assignee: BioValence Sdn. Bhd., Petaling, Jaya (MY)

(21) Appl. No.: 14/410,353

(22) PCT Filed: Jun. 25, 2013
(86) PCT No.: PCT/MA2013/000114

§ 371 Date: Dec. 22, 2014

(30) Foreign Application Priority Data
Jun. 26, 2012 (MY) PI 2012002923

(51) Int. Cl. C07K 14/47; C07K 7/08

(52) U.S. Cl. CPC C07K 14/4723; C07K 14/4705; C07K 7/08; C07K 2319/00; A61K 38/00

(57) ABSTRACT

Use of at least one fusion protein comprising at least one Type 1 Ribosome Inactivating Protein, polypeptide B; and at least one polypeptide A which is a antimicrobial peptide and/or at least one Cationic AntiMicrobial Peptide, polypeptide C for the preparation of a medicament for treating a cancer and/or a microbial infection wherein the medicament is suitable for oral administration pre food intake.

### FIELD OF THE INVENTION

The present invention relates to dosage regimes of fusion polypeptides, and fragments thereof. In particular, the present invention relates to the oral administration of the fusion polypeptides relative to food intake.

### BACKGROUND TO THE INVENTION

There are several pharmacological differences between drugs, which are of a chemical nature, and that which are made of proteins. One main difference is that protein drugs usually have to be administered parenterally i.e. by injection that include intravenous, intraarterial, intracardiac, intraspinal or intrathecal, intramuscular, intrasynovial, intracutaneous or intradermal and subcutaneous means as well as topical, intranasal and intravitreal means. On the other hand, any possible route including oral administration may administer chemical drugs. Where a disease necessitates regular injections that it be painful and traumatic for the patient after some time.

Oral delivery of proteins to the gastrointestinal tract is known to be problematic as a result of poor protein stability due to both gastric pH and proteolytic digestion by enzymes such as pepsin and trypsin, as well as poor absorption as it is not always easy to cross the epithelial intestinal barrier. Many attempts have been made to deliver proteins by the oral route or locally to the stomach. These attempts usually use nanoparticles containing chitosan to improve the absorption and stability of proteins in the gastrointestinal (GI) tract or crystalline proteins to avoid degradation in the stomach and for treatment of diseases of the GI tract. These methods however, can be either too troublesome or too expensive for manufacturing thus making the cost of the final drug inaccessible to the user.

It is also well known in the art that peptide delivery systems may include the use of polysaccharide/liposaccharide conjugates such as chitin and dextrans, carboxylated chitosans, lipopeptides such as TRIS-glycine-Tripalmitate, liposomes that are basically phospholipid vesicles, PEG-conjugates of polyethylene glycol, adhesive proteins such as laminin and fibronectin as peptide carriers, PEA microspheres, PLA microspheres and the like. The use of these systems increases circulation half-life of the protein drug by up to 50-fold and allow for oral administration. But again some of these systems have some risks of adverse reactions and also some of these systems make the final cost of the drug high and may be cumbersome to exploit as opposed to the use of a straightforward naked protein drug.

Further, because the limit of glomerular filtration is estimated to be 50-70 kDa, renal elimination of larger entities may prove problematic leading to potential toxicity. Also, most protein pharmaceuticals exhibit a short half-life measured in minutes and high clearances because they are usually smaller than 30 kDa. There is thus almost no known naked protein drug that has been formulated for oral administration.

The understanding of biologic proteins is still in its infancy and it has been proposed that pharmacokinetic studies will drive the discovery of new biotherapeutic methodologies. Thus, there remains a need for pharmaceutical formulations, unit dosage forms and/or regimes that can improve stability of a protein in the fed stomach.

### SUMMARY OF THE INVENTION

The present invention is defined in the appended independent claims. Some optional features of the present invention are defined in the appended dependent claims.

According to one aspect of the present invention, there is provided a use of a fusion protein comprising at least one polypeptide B which is a Type 1 Ribosome Inactivating Protein (RIP) or fragment thereof; and
(i) at least one polypeptide A which is an antimicrobial peptide; and/or
(ii) at least one, polypeptide C which is a Cationic Antimicrobial Peptide (CAP) or fragment thereof
   for the preparation of a medicament suitable for oral administration wherein the medicament is for treating a microbial infection and/or cancer in a subject and the medicament is for administration before food intake of the subject.

The polypeptide A may be a type of defensin. In particular, the defensin may be an alpha, beta, theta or big defensin, an analogue, or a fragment thereof.

According to one aspect aspect of the present invention, there is provided a method of improving the oral delivery of at least one peptide to a subject, the method comprising the step of linking or tethering the peptide to a MAP30 protein. The peptide may have antimicrobial and/or anticancer activity.

According to a further aspect of the present invention, there is provided a fusion protein according to any aspect of the present invention for oral administration and for use in treatment of a microbial infection and/or cancer, wherein the fusion protein is to be administered to the subject before food. In particular, the fusion protein may be administered at least an hour before food.

According to another aspect of the present invention, there is provided a method of treating and/or preventing microbial infection and/or cancer in a subject, the method comprising a step of oral administration of an effective amount of the fusion protein according to any aspect of the present invention before food intake. In particular, the fusion protein may be administered at least an hour before food.

As will be apparent from the following description, preferred embodiments of the present invention allow for a fusion protein with an optimal effectiveness with a broad spectrum therapy and/or allowing oral delivery of the protein as some of the several applications.

### BRIEF DESCRIPTION OF THE FIGURES

Preferred embodiments of the fusion protein will now be described by way of example with reference to the accompanying figures in which:

FIG. 1 is a translation map of RetroMAD1 (SEQ ID NO:1 and SEQ ID NO:2).

FIG. 2 is a gel image showing A) Time course expression and B) Solubility of RetroMAD1 expression in E. Coli BL21(DE3) cells. Cells harbouring pRMD were harvested before induction (0 h), and after induction for 1 h, 2 h and 3 h represents the pellet phase, the hours with asterisk (*) represents the supernatant phase. Proteins were analysed on a 15% SDS-PAGE. M: PageRuler™ Protein Ladder Fermentas, U: uninduced, IND: induced and IB: purified inclusion bodies. Arrow indicates E. coli produced RetroMAD1 (41.2 kDa)

FIGS. 3A and B are standard curves to determine the concentration of RetroMAD1 in cat serum using capture ELISA.

FIG. 4A is a graph showing the concentration of RetroMAD1 in the serum of control and treated mice derived from capture ELISA.

FIG. 4B is a graph showing the triplicate data confirming the excellent conformity of results used to derive RetroMAD1 concentration in the serum in FIG. 6(A).

FIG. 5A-C are graphs showing the concentration of RetroMAD1 in the serum of control and treated Guinea Pig serum (A), Small Intestine (B) and Stomach (C) against time

FIG. 6A-D are images of SDS-page results showing Day 1 (A), Day 3 (B), Day 7 (C) and Day 30 (D) thermostability of RetroMAD1.

FIG. 7A is SDS-page results showing the 6th month thermostability of RetroMAD1 in various temperatures.

FIG. 7B is SDS-page results showing the 6th Month thermostability with various temperatures, using β-mercaptoethanol (BME) as reducing agent onto RetroMAD1. In this SDS PAGE, sample of same stock from −20° C. was introduced as a control as well as sample from 4° C.

FIG. 8 is a pathway map showing the ability of RetroMAD1 to up-regulate and down-regulate cellular pathways in normal and virally infected cells.

FIG. 9A-D are graphs showing the ability of RetroMAD1 to significantly inhibit the Dengue Fever Virus NS2B-NS3 polyprotein protease. (A) NS2B-NS3 protease inhibition by RetroMAD1: IC50=4.986±0.629 μM, Ki˜2.49 μM; (B) NS2B-NS3 protease inhibition by (01) (SEQ ID NO:34): IC50=4.740±0.173 μM, Ki˜2.37 μM.

FIG. 10A-D are images of SDS-page proteolytic digestion of RetroGAD1 with pepsin (pH2), trypsin (pH8) and chymotrypsin (pH8) for 1 hour (A), 2 hours (B), 3 hours (C) and 4 hours (D) at 37° C. Sample without presence of enzymes and pre-dissolved RetroGAD1 (stock) were used as negative controls (no digestion). 20 uL of each protein sample with 4× sample buffer was loaded onto SDS-PAGE gels and fragments of protein was analysed.

FIG. 11A-D are images of SDS-page proteolytic digestion of Amatilin with pepsin (pH2), trypsin (pH8) and chymotrypsin (pH8) for 1 hour (A), 2 hours (B), 3 hours (C) and 4 hours (D) at 37° C. Sample without presence of enzymes and pre-dissolved Amatilin (stock) were used as negative controls (no digestion). 20 uL of each protein sample with 4× sample buffer was loaded onto SDS-PAGE gels and fragments of protein was analysed.

FIG. 12A-D are results of SDS-page proteolytic digestion of Tamapal1 with pepsin (pH2), trypsin (pH8) and chymotrypsin (pH8) for 1 hour (A), 2 hours (B), 3 hours (C) and 4 hours (D) at 37° C. Sample without presence of enzymes and pre-dissolved Tamapal1 (stock) were used as negative controls (no digestion). 20 uL of each protein sample with 4× sample buffer was loaded onto SDS-PAGE gels and fragments of protein was analysed.

FIG. 13 is an image of SDS-page proteolytic digestion of RetroMAD1 with pepsin (pH2), trypsin (pH8) and chymotrypsin (pH8) for 1 hour, 2 hours and 3 hours at 37° C. Sample without presence of enzymes and pre-dissolved RetroMAD1 (stock) were used as negative controls (no digestion). 20 uL of each protein sample with 4× sample buffer was loaded onto SDS-PAGE gels and fragments of protein was analysed.

FIG. 14A-E are images of SDS-page results showing Day 1 (A), Day 7 (B), Day 30 (C), Day 1, 7, 30 at 60° C. (D) and Day 90 (E) thermostability of RetroMAD1 (temperatures stated on the top of image and the different time points stated on the bottom of the wells). Protein ladder is the molecular weight markers; sample incubated at −20° C. is the control for respective drugs; BME is 2×β-mercaptoethanol, each sample is loaded with (+) or without (−) BME.

FIG. 15A-D are images of SDS-page results showing Day 1 (A), Day 7 (B), Day 1 and 7, at 50° C. (C) and Day 30 (D) thermostability of RetroGAD1 (temperatures stated on the top of image and the different time points stated on the bottom of the wells). Protein ladder is the molecular weight markers; sample incubated at −20° C. is the control for respective drugs; BME is 2× β-mercaptoethanol, each sample is loaded with (+) or without (−) BME.

FIG. 16A-D are images of SDS-page results showing Day 1 (A), Day 7 (B), Day 1 and 7, at 50° C. (C) and Day 30 (D) thermostability of Amatilin (temperatures stated on the top of image and the different time points stated on the bottom of the wells). Protein ladder is the molecular weight markers; sample incubated at −20° C. is the control for respective drugs; BME is 2×β-mercaptoethanol, each sample is loaded with (+) or without (−) BME.

FIG. 17A-D are images of SDS-page results showing Day 1 (A), Day 7 (B), Day 1 and 7, at 50° C. (C) and Day 30 (D) thermostability of Tamapal1 (temperatures stated on the top of image and the different time points stated on the bottom of the wells). Protein ladder is the molecular weight markers; sample incubated at −20° C. is the control for respective drugs; BME is 2×β-mercaptoethanol, each sample is loaded with (+) or without (−) BME.

FIG. 18 are gel images of showing the stability of fusion proteins, RetroMAD1, RetroGAD1, Amatilin and Tamapal1: A1 and A2 are RetroMAD1 subjected to temperature fluctuations; B1 and B2 are RetroGAD1 subjected to temperatures; C1 and C2 are Amatilin subjected to temperature fluctuations; D1 and D2 are Tamapal1 subjected to temperature fluctuations. Protein Ladder is the marker for protein size; Control is untreated drug; T1-4 are the different temperature fluctuations (as shown in Table 6) BME is 2×β-mercaptoethanol, the samples are loaded with (+) or without (−) BME.

FIG. 19A-C are graphs showing the percentage of viral reduction caused by Amatilin (A), RetroGAD1 (B) and Tamapal1 (C) incubated at different temperatures for 1, 7 and 30 days in simultaneous treatment determined by PCR. * Thermostability was not tested for 50° C. for 30 days incubation

FIG. 20 is a graph showing the percentage of viral reduction caused by Amatilin, RetroGAD1 and Tamapal1 exposed to various temperature fluctuations in simultaneous treatment determined by PCR.

FIG. 21A-D are graphs showing the inhibition of NS2B-NS3 by various drugs. (A) Inhibitory activity of RetroMAD1 against NS2B-NS3; (B) inhibitory activity of RetroGAD1 against NS2B-NS3; (C) inhibitory activity of Amatilin against NS2B-NS3; and (D) inhibitory activity of Tamapal1 against NS2B-NS3.

FIG. 22A-D are graphs showing the concentration of RetroMAD1 (A), RetroGAD1 (B), Amatilin (C) Tamapal1 (D) (μg/ml) in mice blood serum after oral administration of RetroMAD1 (A), RetroGAD1 (B), Tamapal1 (C) at 0.5, 1, 2, 4, 8, 12 hours for Day 1 and 30 minutes post feeding for Day 2, Day 3, Day 4, Day 5, Day 6, Day 7 and Day 10.

FIG. 23A-D are graphs showing the concentration of RetroMAD1 (A), RetroGAD1 (B), Amatilin (C) and Tamapal1 (D) (μg/ml) in stomach, liver, intestine and kidney against Time

FIGS. 24A-D are graphs showing concentration of RetroMAD1 (μg/ml) (A), RetroGAD1 (μg/ml) (B), Amatilin (μg/ml) (C), Tamapal1 (μg/ml) (D) leached out against Time (minutes)

FIG. 25 is a graph showing concentration of RetroMAD1 in hepatopancreas, tail muscle, faeces and control against time in a short-term pharmacokinetics study

FIG. 26 is a graph showing concentration of RetroMAD1 in hepatopancreas, tail muscle, faeces and control against time in a long-term pharmacokinetics study

FIG. 27 is a schematic diagram of the process that may be used for Supercritical Fluid Drying (SCFD)

FIG. 28 is an SEM picture of RetroMAD1 crystals

FIG. 29 is a graph showing the percentage of viral reduction caused by RetroMAD1 micronized powder in simultaneous treatment determined by PCR.

FIG. 30 is an image of protein profile of RetroMAD1 against HSV2; cells as control, cells treated with RetroMAD1, Cells infected with HSV2 and HSV2 infected cells treated with RetroMAD1.

FIG. 31 is the pathway of HSV2 infection in cells (i) Entry (ii) Uncoating and nuclear transport (iii) Replication (iv) Translation (v) Transport to cytoplasm and (vi) Egress. Proteins involved are mainly in viral entry, replication and translation.

FIG. 32 is an image of protein profile of RetroMAD1 against DENV2; cells as control, cells treated with RetroMAD1, Cells infected with HSV2 and HSV2 infected cells treated with RetroMAD1.

FIG. 33 is the pathway of DENV2 infection in cells (i) Entry (ii) Uncoating and nuclear transport (iii) Replication (iv) Translation (v) Transport to cytoplasm and (vi) Egress. Proteins involved are mainly in viral entry, replication and translation.

### DETAILED DESCRIPTION OF THE INVENTION

For convenience, certain terms employed in the specification, examples and appended claims are collected here.

The term “adjuvant”, as used in the context of the invention refers to an immunological adjuvant. By this, an adjuvant is meant to be a compound that is able to enhance or facilitate the immune system's response to the ingredient in question, thereby inducing an immune response or series of immune responses in the subject. The adjuvant can facilitate the effect of the therapeutic composition by forming depots (prolonging the half-life of the ingredient), provide additional T-cell help and stimulate cytokine production. Facilitation of antigen survival and unspecific stimulation by adjuvants may, in some cases, be required if the antigenic molecule are only weakly antigenic or only exerts weak to moderate interactions with compounds, molecules, or cells of the immune system.

The term “analogue” as used in the context of the invention refers to a peptide that may be modified by varying the amino acid sequence to comprise one or more naturally-occurring and/or non-naturally-occurring amino acids, provided that the peptide analogue is capable of reducing or preventing growth of a microorganism or killing a microorganism. For example, the term “analogue” encompasses an inhibitory peptide comprising one or more conservative amino acid changes. The term “analogue” also encompasses a peptide comprising, for example, one or more D-amino acids. Such an analogue has the characteristic of, for example, protease resistance. Analogues also include peptidomimetics, e.g., in which one or more peptide bonds have been modified. Preferred analogues include an analogues of a peptide as described according to any embodiment here comprising one or more non-naturally-occurring amino acid analogues.

The term “antimicrobial”, as used in the context of the invention refers to the biological activity of the peptide or analogue or derivative thereof of the present invention, and means that the proteins of the present invention have the capacity to kill, disrupt reproduction or otherwise disable microbial growth. The peptide or analogue or derivative thereof of the present invention is capable of killing a microorganism and/or reducing or preventing growth of a microorganism.

i.e., the peptide has microbicidal activity and/or microbiostatic activity. The peptide may be a drug, compound or molecule, including the fused protein according to any embodiment of the present invention for use in treating or preventing microbial infection. Methods for determining the antimicrobial activity of a peptide or analogue or derivative thereof will be apparent to a skilled person and/or described herein. For example, the peptide or analogue or derivative is applied to a substrate upon which a microorganism has been previously grown and, after a suitable period of time, the level of growth inhibition and/or cell death of the microorganism are determined. Polypeptide A is a non limiting example of an antimicrobial peptide. Polypeptide A may be a theta defensin, an analogue, or a fragment thereof and the like.

The term “comprising” as used in the context of the invention refers to where the various components, ingredients, or steps, can be conjointly employed in practicing the present invention. Accordingly, the term “comprising” encompasses the more restrictive terms “consisting essentially of” and “consisting of.” With the term “consisting essentially of” it is understood that the epitope/antigen of the present invention “substantially” comprises the indicated sequence as “essential” element. Additional sequences may be included at the 5′ end and/or at the 3′ end. Accordingly, a polypeptide “consisting essentially of” sequence X will be novel in view of a known polypeptide accidentally comprising the sequence X. With the term “consisting of” it is understood that the polypeptide, polynucleotide and/or antigen according to the invention corresponds to at least one of the indicated sequence (for example a specific sequence indicated with a SEQ ID Number or a homologous sequence or fragment thereof).

The term “derivative” as used in the context of the invention includes e.g., a fragment or processed form of the stated peptide, a variant or mutant comprising one or more amino acid substitutions, deletions of additions relative to the stated peptide, a fusion protein comprising the stated peptide or a peptide comprising one or more additional non-peptide components relative to the stated peptide e.g., a chemical component, e.g., polyethylene glycol (PEG). The term “derivative” also encompasses polypeptides comprising the fusion protein according to the invention. For example, the polypeptide comprises a label, such as, for example, an epitope, e.g., a FLAG epitope or a V5 epitope or an HA epitope. For example, the epitope is a FLAG epitope. Such a tag is useful for, for example, purifying the polypeptide. A preferred derivative of an antimicrobial fusion protein of the invention has enhanced stability. For example, a cleavage site of a protease active in a subject to which a fusion protein is to be administered is mutated and/or deleted to produce a stable derivative of an antimicrobial fusion protein of the invention. The term “derivative” also encompasses a derivatized peptide, such as, for example, a peptide modified to contain one or more-chemical moieties other than an amino acid. The chemical moiety may be linked covalently to the peptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound.

Accordingly, acceptable amino acid substitutions are generally therefore based on the relative similarity of the amino acid side-chain substituents, for example, their hydrophobicity, hydrophilicity, charge, size, and the like. Exemplary substitutions which take several of the foregoing characteristics into consideration are well known to those of skill in the art and include: arginine and lysine; glutamate and aspartate; serine and threonine; glutamine and asparagine; and valine, leucine and isoleucine. The isolated peptides of the present invention can be prepared in a number of suitable ways known in the art including typical chemical synthesis processes to prepare a sequence of polypeptides.

The term “fragment” as used in the context of the invention refers to an incomplete or isolated portion of the full sequence of the fusion protein according to any aspect of the present invention which comprises the active site(s) that confers the sequence with the characteristics and function of the protein. In particular, it may be shorter by at least one amino acid. For example a fragment of the fusion protein according to the present invention comprises the active site(s) that enable the protein to recognise a microorganism. The fragment may at least be 10 amino acids in length. For example, a non-limiting fragment of RIP may at least comprise the core or the bioactive site of the RIP which may be approximately 5 kDa in size.

The term “fusion protein(s)” as used in the context of the invention refers to proteins created through the joining of two or more genes, which originally coded for separate proteins. Translation of this fusion gene results in a single polypeptide with functional properties derived from each of the original proteins. Recombinant fusion proteins are created artificially by recombinant DNA technology for use in biological research or therapeutics. For example, the fusion protein according to any aspect of the present invention may comprise a Type 1 RIP, polypeptide B; and a polypeptide A capable of viral entry inhibition and/or a CAP, polypeptide C. The structure of the fusion protein may be A-B-C, A-C-B, C-A-B, C-B-A, B-A-C, B-C-A, A-B-C-C, A-B, B-C, B-C-C or C-C-B-C-C. In particular, the fusion protein may comprise dimers and/or tandem repeats. More in particular, the structure of the fusion protein according to any aspect of the present invention may be repeats of the structure mentioned above. For example, the structure may be A-A-B-C-C, C-C-B-C-C, A-A-B-A-A and the like. The polypeptide A, B or C in each fusion protein may be the same protein or may be a different protein when repeated. Polypeptide A may be theta defensin, an analogue, or a fragment thereof. A fusion protein according to the present invention may comprise the sequence of SEQ ID NO:1, a variant, derivative or fragment thereof. The term “RetroMAD1” is used in the present invention to refer to a fusion protein with the structure A-B-C and with amino acid sequence SEQ ID NO:1. In particular, in RetroMAD1 polypeptide A may be Retrocyclin 101, polypeptide B may be MAP30 and polypeptide C may be Dermaseptin 1. These peptides may be directly fused to one another or connected to one another by a linker peptide.

The term “linker peptide”, as used in the context of the invention is used interchangeably with the term “linker” herein. A linker peptide is a peptide that covalently or non-covalently connects two or more molecules or peptides, thereby creating a larger complex consisting of all molecules or peptides including the linker peptide. A non-limiting example of a linker peptide may be SEQ ID NO:3.

The term “microbial infection” as used in the context of the invention refers to the invasion, development and/or multiplication of a microorganism within or on another organism. A microbial infection may be localized to a specific region of an organism or systemic. Infections for which a fusion peptide, analog and/or derivative of the invention are useful for treating include any infection which affects mammals, invertebrates, vertebrates and/or plants, caused by any microorganism, for example but not limited to bacteria, fungi, yeasts, protozoa and viruses. The infection may include a protist infection or a rogue cell line. A skilled person would understand what is considered a microbial infection. In particular, a fusion protein or analogue or derivative or formulation of the present invention is useful for treating an infection by a virus.

Examples of viruses include but are not limited to measles virus, herpes simplex virus (HSV-1 and -2), herpes family members (HIV, hepatitis C, vesicular stomatitis virus (VSV), visna virus, cytomegalovirus (CMV) and the like.

The term “microorganism” as used in the context of the invention encompasses any microscopic organism or microbe. For example, but not limiting, the microorganism includes a bacterium, an archaebacterium, a virus, a yeast, a fungus or a protist. In particular, the microorganism is a virus. The virus may include but are not limited to, cytomegalovirus (CMV) pneumonia, enteritis and retinitis; Epstein-Barr virus (EBV) lymphoproliferative disease; chicken pox/shingles (caused by varicella zoster virus, VZV); HSV-1 and -2 mucositis; HSV-6 encephalitis, BK-virus hemorrhagic cystitis; viral influenza; pneumonia from respiratory syncytial virus (RSV); AIDS (caused by HIV); and hepatitis A, B or C. Additional examples of viruses include but are not limited to Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astro viruses).

For example, the viruses may be specific to aquaculture such as but not limited to Crustacean viruses such as WSSV, HPV, MBV, IHHNV, YHV, TSV, GAV, LSNV, IMNV, MoV, KHV1, KHV2, KHV3, VNN. The viruses specific to aquaculture may include fish viruses from any one of the family of Birnaviridae, Herpesviridae, Iridoviridae, Retroviridae or Rhabdoviridae. In particular, the fish viruses may be pancreatic necrosis virus (IPNV) from the Birnaviridae family, channel catfish virus (CCV) from the Herpesviridae family, fish lymphocystis disease virus (FLDV) from the Iridoviridae family, hematopoietic necrosis virus (IHNV) and viral hemorrhagic septicemia virus (VHSV) belonging to the Rhabdoviridae family and the like. Abalone viruses include AVG, AMAV and the like.

For example, the viruses may be specific to poultry such as but not limited to viruses that cause avian pox, Newcastle disease, infectious bronchitis, quail bronchitis, Marek's Disease (Visceral Leucosis), Lymphoid Leucosis, Infectious Bursal Disease, avian influenza, epidemic tremor and the like.

For example, the viruses may be specific to pigs such as but not limited to swine hepatitis E virus, Circoviruses, Herpesviruses and the like. In particular, the viruses may be Porcine cytomegalovirus, pseudorabies virus.

Viruses significant to cats include but are not limited to Feline Panleukopenia virus (FPV), Feline herpesvirus, Feline calicivirus, Feline Leukemia Virus (FeLV), Feline Immunodeficiency Virus (FIV) and the like. The viruses may be specific to dogs and these may include but are not limited to Rabies virus, canine parvovirus, canine coronavirus, canine distemper virus, canine influenza, canine hepatitis virus, canine herpesvirus, a virus that causes pseudorabies, canine minute virus and the like.

A virus may include a bacteriophage, also known as a phage that includes a group of viruses that infect specific bacteria, usually causing their disintegration or dissolution. A bacteriophage may be selected from a group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, Tectiviridae and the like. In particular, the phage may be Lambda phage (A phage)—lysogen (λ phage), T2 phage, T4 phage, T7 phage, T12 phage, R17 phage, M13 phage, MS2 phage, G4 phage, P1 phage, Enterobacteria phage P2, P4 phage, Phi X 174 phage, N4 phage, Pseudomonas phage φ6, φ29 phage, 186 phage and the like.

A bacteria may include Aeromonas hydrophila, Aeromonas salmonicida, Aeromonas sobrio, Enterobacter aerogenes, Enterococcus faecalis, Escherichia coli, Flavobacterium meningosepticum, Helicobacter pylori, Klebsiella pneumonia, Listeria monocytogenes, Listonella anguillarum, Methicillin-resistant Staphylococcus aureus, Micrococcus luteus, Morganella morganii, Pasturella multocida, Pseudomonas aeruginosa, Salmonella typhimurium, Staphylococcus aureus, Staphylococcus epidermidis, Staphylococcus haemolyticus, Streptococcus agalactiae, Streptococcus equi, Streptococcus iniae, Streptococcus uberis, Vibrio alginolyticus, Vibrio anguillarum, Vibrio cholera, Vibrio damsel, Vibrio fluvialis, Vibrio furnissi, Vibrio harveyi, Vibrio hollisae, Vibrio metschnikovii, Vibrio mimicus, Vibrio parahaemolyticus, Vibrio proteolyticis, Vibrio vulnificus, Vibrio splendidus, Yersinia ruckeri and the like.

The term “polypeptide” as used in the context of the invention may refer to a long, continuous, and unbranched peptide and may include cyclic polypeptides. Proteins consist of one or more polypeptides arranged in a biologically functional way and may often be bound to cofactors, or other proteins. In particular, the protein according to any aspect of the present invention may be naturally occurring, de novo and/or synthetic.

The term “subject” as used in the context of the invention refers to any animal, including a human, non-human animal, plant or insect that may be infected by a microorganism. In particular, the subject is any animal, including a human, plant or insect that may be infected by a microorganism against which a fusion protein or analogue or derivative of the invention is active. More in particular, the subject may be a non-aquatic animal. For example, a prawn, fish, crustacean, etc.

The term “treatment”, as used in the context of the invention refers to prophylactic, ameliorating, therapeutic or curative treatment.

The term “tumour” or “cancer”, as used in the context of the invention refers to an abnormal mass of tissue as a result of abnormal proliferation of cells. The term “tumour” refers to a mass of cells, which may not necessarily be cancer. Cancer is a type of malignant tumour. The term “tumour” or “cancer” as used herein may be used to describe a disease selected from the group consisting of Non-Hodgkin's Lymphoma, brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, pontine glioblastoma, Kaposi's sarcoma, or any other type of solid or liquid cancer.

The term “variant”, as used in the context of the invention can alternatively or additionally be characterised by a certain degree of sequence identity to the parent polypeptide from which it is derived. More precisely, a variant in the context of the present invention exhibits at least 30% sequence identity, in particular at least 40%, 50%, 60%, 70%, 80% or 90% sequence identity. More in particular, a variant in the context of the present invention exhibits at least 95% sequence identity to its parent polypeptide. The variants of the present invention exhibit the indicated sequence identity, and preferably the sequence identity is over a continuous stretch of 100, 150, 200, 300, 315, 320, 330, 340, 344 or more amino acids. The similarity of nucleotide and amino acid sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL available e.g. on http://www.ebi.ac.uk/Tools/clustalw/. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). Preferably, sequence matching analysis may be supplemented by established homology mapping techniques like Shuffle-LAGAN (Brudno M., Bioinformatics 2003b, 19 Suppl 1:154-162) or Markov random fields. When percentages of sequence identity are referred to in the present application, these percentages are calculated in relation to the full length of the longer sequence, if not specifically indicated otherwise.

A person skilled in the art will appreciate that the present invention may be practiced without undue experimentation according to the method given herein. The methods, techniques and chemicals are as described in the references given or from protocols in standard biotechnology and molecular biology text books.

In one aspect of the present invention, there is provided a use of a fusion protein comprising at least one polypeptide B which is a Ribosome Inactivating Protein (RIP) or fragment thereof; and
(i) at least one polypeptide A which is an antimicrobial peptide capable of viral entry inhibition; and/or
(ii) at least one polypeptide C which is a Cationic AntiMicrobial Peptide (CAP) or fragment thereof
   for the preparation of a medicament suitable for oral administration wherein the medicament is for treating a microbial infection and/or cancer.

The medicament may be administered before food intake or during food intake. In particular, the medicament may be administered before food intake in the subject. The medicament may be administered at least 15 minutes before food intake. In particular, the medicament may be administered ≧20 mins, 30 mins, 40 mins, 45 mins, 50 mins, 55 mins, 60 mins, 65 mins, 75 mins, 80 mins, 90 mins, 95 mins, 100 mins or 120 mins before feeding and/or food intake.

The configuration of the medicament may be considered a tethering configuration where a bioactive tethering polypeptide may be attached to bioactive polypeptide payloads to form a naked fusion protein drug of the present invention to enable rapid appearance within the serum post-oral feeding with pharmacokinetic evidence. The medicament of the present invention are shown to be resistant to both pepsin and trypsin digestion for an extended period of time sufficient to survive the GI tract. These medicaments may be considered heat stable and do not require expensive cold-chain transportation. In particular, these medicaments and/or fusion proteins of the present invention may be micronized into a free flowing powder with high process yields to enable further ease of oral drug delivery in tablet and/or capsule form.

This dosage regime allows for a peptide according to any aspect of the present invention that may be larger than 30 kDa but smaller than 50 kDa to exhibit the potential for oral delivery which means it may be able to withstand proteolysis by the main digestive enzymes such as pepsin, trypsin and chymotrypsin at their respective pH optima, and above that to exhibit a half-life in hours rather than minutes and finally to exhibit broad-spectrum antiviral activity.

This may allow for the fusion protein according to any aspect of the present invention to be industrially useful as a class of recombinant protein compounds that not only has broad-spectrum antiviral capabilities but are also able to be conveniently used and thus accessible to the general public as they may be orally administered. No complicated modification, encapsulation, conjugation, treatment and formulation may thus be required. The members of this class of proteins may be between 35-45 kDa making them not too small as to have too short a retention time as in the case of proteins below 30 kDa and not too large as to have problems with renal elimination as in the case of proteins above 50 kDa.

Administering via oral delivery is preferred over any other route because of simplicity and convenience. Low permeability, low lipophilicity and inactivation in the GI tract by its digestive enzymes are the main obstacles. Out of 9 new technologies being developed to allow proteins to be orally administered, 6 of these technologies focus on protection against digestive enzyme activity. Oral delivery of protein drugs may also be a future driver for personalized medicine.

Nanoparticle encapsulation and covalent modification with glycosylphosphotidylinositol (GPI) may be used for oral delivery of the fusion proteins according to any aspect of the present invention into the subject.

The medicament may be for administration with a drink. The drink may include water, pure water, flavoured and the like. For example, since RetroMAD1 according to the present invention may maintain a peak at 1-2 hrs before tapering off at 4 hrs, and may clear out of the system at about 12 hrs may be considered significant retention time that may be amenable to dosage regimes that are planned 30 min-1 hr before food, administered diluted with water as a drink, and administered 2-3 times per day depending upon the severity of the viral infection or cancer.

The medicament may be administered via a medicated chewing gum.

A fusion protein according to the first aspect can also comprise a variant or a derivative. The terms “variant” and “derivative” are defined above.

Polypeptide A may be at least one peptide with antimicrobial activity. In particular, the polypeptide A may be a peptide with microbial entry inhibition activity. More in particular, the polypeptide A may be a may be a defensin, an analogue, or a fragment thereof. Even more in particular, the defensin may be an alpha, a beta, a theta or a big defensin, an analogue, or a fragment thereof. In particular, polypeptide A may be a Retrocyclin, polypeptide B may be MAP30 and polypeptide C may be a Dermaseptin. More in particular, polypeptide A may be Retrocyclin 101 (RC101) and polypeptide C may be Dermaseptin 1. A polypeptide comprising RC101, MAP30 and Dermaseptin 1 as polypeptide A, B and C respectively is termed RetroMAD1 in the present invention. RetroMAD1 may exhibit significant viral copy reduction in cell challenge assays for HSV1, HSV2, DEN1, DEN2, DEN3 and DEN4 viruses in pre-treatment, simultaneous and post-treatments as ascertained by RT-PCR.

In particular, polypeptide A may comprise amino acid sequence with SEQ ID NO: 4, a fragment or variant thereof, polypeptide B may comprise amino acid sequence with SEQ ID NO:5, a fragment or variant thereof, and polypeptide C may comprise amino acid sequence with SEQ ID NO:6, a fragment or variant thereof.

Polypeptide B may be a Ribosome Inactivating Protein (RIP) or fragment thereof. More in particular, polypeptide B may be a Type I RIP or fragment thereof. In particular, the type 1 RIP may be selected from the group consisting of α-Ebulitin, β-Ebulitin, γ-Ebulitin, Nigritin f1, Nigritin f2, Amarandin-S, Amaranthus antiviral/RIP, Amarandin-1, Amarandin-2, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparin 1, Asparin 2, Agrostin, Dianthin 29, DAP-30, DAP-32, Dianthin 30, Dianthus chinensis RIP1, Dianthus chinensis RIP2, Dianthus chinensis RIP3, Lychnin, Petroglaucin, Petrograndin, Saponaria ocymoides RIP, Vacuolas saporin, Saporin-1, Saporin-2, Saporin-3, Saporin-5, Saporin-6, Saporin-7, Saporin-9, Vaccaria hispanica RIP, Benincasin, α-benincasin, β-benincasin, Hispin, Byrodin I, Byrodin II, Colocin I, Colocin 2, Cucumis figarei RIP, Melonin, C. moschata RIP, Cucurmosin, Moschatin, Moschatin I, Moschatin II, Moschatin III, Moschatin IV, Moschatin V, Pepocin, Gynostemmin I, Gynostemmin II, Gynostemmin III, Gynostemmin IV, Gynostemmin V, Gynostemma pentaphyllum RIP, Gypsophilin, Lagenin, Luffaculin, Luffangulin, Luffin-alpha, Luffin-B, MOR-I, MOR-II, Momordin II, Alpha-momorcharin, β-momorcharin, γ δ-momorcharin, γ-momorcharin, Momorcochin, Momorcochin-S, Sechiumin, Momorgrosvin, Trichoanguin, α-kirilowin, β-kirilowin, α-trichosanthin, TAP-29, Trichokirin, Trichomislin, Trichosanthin, Karasurin-A, Karasurin-B, Trichomaglin, Trichobakin, Crotin 2, Crotin 3, Euserratin 1, Euserratin 2, Antiviral Protein GAP-31, Gelonin, Hura crepitans RIP, Curcin, Jathropa curcas RIP, Mapalmin, Manutin 1, Manutin 2, α-pisavin, Charibdin, Hyacinthus orientalis RIP, Musarmin 1, Musarmin 2, Musarmin 3, Musarmin 4, Iris hollandica RIP, Cleroendrum aculeatum RIP, CIP-29, CIP-34, Crip-31, Bouganin, Bougainvilla spectbilis RIP, Bougainvillea×buttiana Antiviral protein 1 (BBAP1), malic enzyme 1 (ME1), ME2, MAP-S, pokeweed antiviral protein (PAPa-1), PAPa-2, PAP-alpha, PAP-I, PAP-II, PAP-S, PD-SI, DP-S2, Dodecandrin, Anti-viral protein PAP, PIP, PIP2, Phytolacca octandra anti-viral protein, Phytolacca, octandra anti-viral protein II, Hordeum vulgare RIP-I, Hordeum vulgare RIP-II, Hordeum vulgare sub sp. Vulgare Translational inhibitor II, Secale cereale RIP, Tritin, Zea, diploperemis RIP-I, Zea diploperemis RIP-II, Malus×domestica RIP, Momordica Anti-HIV Protein (MAP30), Gelonium multiflorum (GAP31), pokeweed antiviral protein (PAP), Mirabilis expansa 1 (ME1), malic enzyme 2 (ME2), Bougainvillea×buttiana antiviral protein 1 (BBAP1), phage MU1, betavulgin (Bvg), curcin 2, saporin 6, Maize RIP (B-32), Tobacco RIP (TRIP), beetin (BE), BE27, Mirabilis antiviral protein (MAP), Trichosanthin (TCS), α-luffin, α-Momorcharin (α-MMC), (β-MMC luffin, Ocymoidin, Bryodin, Pepopsin, β-trichosanthin, Camphorin, YLP, Insularin, Barley RIP, Tritins, Lamjaring Volvariella volvacea RIP and the like from any plant origin.

Polypeptide C, a Cationic Antimicrobial Peptide (CAP) may be an anti-viral CAP that may play a role in viral fusion inhibition, viral gene suppression, viral membrane disruption and/or viral entry inhibition. CAPs may be a maximum of 100 amino acids in length. CAPs may mostly be of animal origin. However, there may also be CAPs, which are from plants, which include but are not limited to cyclotides. For example, bacteria CAPs which may function as fusion inhibitors may include but are not limited to Siamycin, NP-06 and Gramicidin A. Plant CAPs which may function as fusion inhibitors may include Circulin A, B, Kalata B1 and B8; Plant CAPs which may function as entry inhibitors may include Kalata B8; Plant CAPs which may function as viral gene suppressors may include Ginkbilobin, Alpha-Basrubin, Lunatusin and Sesquin. Plant CAPs which may function as viral membrane disruptors may include Circulin A, C and D, Tricyclon A and Cycloviolacin H4. Animal CAPs which may function as fusion inhibitors may include Polyphemusin I and II, hfl-B5, Protegrin (Pig Cathelicidin), Rat Defensin NP1, NP2, NP3 and NP4, Human β-defensin I and II, Temporin A, Temporin-LTc, Temporin-Pta, Caerin 1.1, Ranatuerin 6 and 9, Reptile Defensin and Piscidin 1 and 2; Animal CAPs which may function as entry inhibitors include Lactoferricin B, Rabbit Neutrophil-1 Corticostatin III a, Rabbit Neutrophil-3A, Rabbit α-Defensin, Retrocyclin-1, Retrocyclin-2, Retrocyclin-3, Human α-Defensin HNP-1, 2, 3, 4, 5 & 6, Human β-defensin III (HBD3), Rhesus minidefensin (RTD-1,θ-defensin), RTD-2 rhesus θ-defensin, RTD-3 rhesus θ-defensin, Human neutrophil peptide-2, Human neutrophil peptide-3 and human neutrophil peptide-4; Animal CAPs which may function as viral gene suppressors: Cecropin A, Melittin, EP5-1, Magainin 2, hepcidin TH1-5, and Epinecidin-1; Animal CAPs which may function as viral membrane disruptors may include Indolicidin, Cathelicidin-4, Human neutrophil peptide-1, LL-37 Cathelicidin, Dermaseptin-S1, S4 and S9, Maximin 1, 3, 4 and 5, Brevinin 1, Ranatuerin 2P, 6 and 9 Esculentin 2P, Esculentin-1 Arb, Caerin 1.1, 1.9 and 4.1, Brevinin-2-related, Maculatin 1.3, Maximin H5 and Piscidin 1 and 2. Other CAPs may include Mundticin KS Enterocin CRL-35, Lunatusin, FK-13 (GI-20 is a derivative), Tachyplesin I, Alpha-MSH, Antiviral protein Y3, Piscidin 3, Palustrin-3AR, Ponericin L2, Spinigerin, Melectin, Clavanin B, Cow cathelicidin BMAP-27, BMAP-28, Guinea pig cathelicidin CAP11, Sakacin 5X, Plectasin, Fungal Defensin, GLK-19, lactoferrin (Lf) peptide 2, Kalata B8, Tricyclon A, Alloferon 1, Uperin 3.6, Dahlein 5.6, Ascaphin-8, Human Histatin 5, Guineapig neutrophil CAP2 & CAP1, Mytilin B & C, EP5-1, and Hexapeptide (synthetic) Corticostatin IV Rabbit Neutrophil 2.

### The Type 1 RIP may:

(i) act as an RNA N-Glycosidase which hydrolyses the N-C glycosidic bond of adenosine at position 4324 of the universally conserved sarcin/ricin domain (S/R domain) of the 28S-rRNA in the eukaryotic ribosome and render it incapable of carrying out protein synthesis thus, functionally, blocking translation,
(ii) act directly on the virus particles or viral nucleic acids by means of their polynucleotide: adenosine glycosidase activity, and/or
(iii) act as a DNA glycosylase/apurinic (AP) lyase capable of irreversibly relaxing HIV-1 supercoiled DNA and catalyzing double-stranded breakage to form inactive products.

The fusion protein according to any aspect of the present invention may be an antimicrobial and/or anticancer compound capable of a broad spectrum of applications and that may be economically produced without any limitation of raw material supply unlike certain antimicrobial and/or anticancer compounds known in the art. The fusion protein according to any aspect of the present invention may thus be economically produced in a large scale without any limitations of raw material supply.

In order to achieve broad-spectrum activity, the fusion peptide according to any aspect of the present invention may be able to interfere with the viral infection and propagation processes in a number of different pathways, that is to say, in viral entry inhibition, viral fusion inhibition, viral integrase inhibition and viral translation inhibition. The fusion peptide may thus have a multidomain and/or multifunctional ability. An entire new class of antiviral drugs may thus be produced from the fusion protein according to any aspect of the present invention. The number of combinations and permutations that may be obtained from expressed polypeptides A, B and C as fusion antiviral proteins potentially numbers in the tens of thousands.

The use of the fusion proteins according to any aspect of the present invention, may involve combining anticancer properties from 2, or more likely 3 genes, to produce potent anticancer chimeric proteins that are capable of oral administration and are stable at room temperature to avoid costly cold-chain transportation. Also, the fusion products according to any aspect of the present invention may have potent antiviral activities that can be useful a significant percentage of human cancers are caused by viral infections. In particular, these fusion products may be capable of inhibition of polyprotein serine proteases as demonstrated by their inhibition of the NS2B NS3 protease of Dengue Virus. Also, these fusion products may be capable of killing HSV-2 as shown in the Examples.

In particular, the fusion protein may comprise at least one formula selected from the group consisting of formulas I-XIII:

A-B-C, Formula I:

A-B-C-C, Formula II:

A-B, Formula III:

A-C-B, Formula IV:

C-A-B, Formula V:

C-B-A, Formula VI:

C-B, Formula VII:

B-A-C, Formula VIII:

B-A-C-C, Formula IX:

B-C-A, Formula X:

B-A-C, Formula XI:

B-C, Formula XII:

B-A, Formula XIII:

C-C-B-C-C, Formula XIV:

C-B-C. Formula XV:

Polypeptide A may be an antimicrobial peptide. In particular, polypeptide A may be an viral entry inhibitory protein. More in particular, polypeptide A may be a defensin, an analogue, or a fragment thereof. Even more in particular, the defensin may be an alpha, a beta, a theta or a big defensin, an analogue, or a fragment thereof, polypeptide B may be Type 1 RIP, or a fragment thereof, polypeptide C may be Cationic AntiMicrobial Peptide (CAP), or a fragment thereof, polypeptide D may be synthetic anticancer sequence; and - may be a direct linkage or a linker peptide.

In particular, the linker peptide may comprise a polypeptide sequence: [VPXVG]ₙ, (SEQ ID NO:3) wherein X is an unknown or other amino acid and n is the number of repeats of SEQ ID NO:3 in each linker peptide. For example, n may be 1, 2, 3, 4 or 5. More in particular, X in SEQ ID NO:3 is G and n is 2.

In another example, the linker peptide may be a glycine-serine linker. In particular, the glycine-serine linker may have a sequence of [G-G-G-S]ₙ (SEQ ID NO:27).

In particular, the fusion protein may comprise the formula I:

A-B-C-

wherein, polypeptide A may be a defensin, an analogue, or a fragment thereof. Even more in particular, the defensin may be an alpha, a beta, a theta or a big defensin. In particular, the polypeptide A may be a theta defensin, an analogue, or a fragment thereof, polypeptide B is Type 1 RIP, or a fragment thereof, and polypeptide C may be a CAP, or a fragment thereof and - may be a direct linkage or a linker peptide.

More in particular, polypeptide A may be fused to polypeptide B via at least one first linker peptide of SEQ ID NO: 3. Even more in particular, polypeptide A may be fused to polypeptide B via a peptide of SEQ ID NO: 3, wherein X is G and n is 2. Polypeptide B may be directly linked to polypeptide C with no linker peptide in-between. Polypeptide C in formula I may comprise a second linker peptide on the free end not linked to B. The second linker peptide may comprise the formula SEQ ID NO: 3. Even more in particular, in the second linker peptide X is G and n is 2.

Polypeptide A may be a viral entry inhibitor protein. Polypeptide A may be a defensin (α, β, θ or big). Defensins are known to be up-regulated in tumors and exhibit anti-angiogenic antitumor effects. In particular, polypeptide A may be a theta Defensin of a vertebrate or invertebrate origin. In particular, theta Defensin may be from a bacterium, fungus, mammal, amphibian or reptile. The mammal may be a non-human primate and/or the invertebrate may be a Horseshoe crab and/or an insect. The theta Defensin may be selected from the group consisting of Rhesus minidefensin (RTD-1), RTD-2, RTD-3, Retrocyclin-1, Retrocyclin-2, Retrocyclin-3 from Macaca mulatta of SEQ ID Nos: 7-12 respectively and the like (Tang Y Q, 1999; Leonava L, 2001; Wang W, 2004).

The theta Defensin may be synthetic and may be selected from a group of retrocyclin congeners RC100-RC108 and RC110-RC114 of SEQ ID NO:13-25 respectively (Cole et. al. 2002: Wang et. al. 2003). The sequences of Retrocyclin (RC) 100-108 and RC110-RC114 are shown in Table 1a below.

TABLE 1a
Polypeptide sequences of natually occurring and
synthetic theta Defensin proteins.

| SEQ ID NO: | Sequences |
|---|---|
| 7 | GFCRCLCRRGVCRCICTR |
| 8 | RCLCRRGVCRCLCRRGVC |
| 9 | RCICTRGFCRCICTRGFC |
| 10 | GICRCICGRGICRCICGR |
| 11 | GICRCICGRGICRCICGR |
| 12 | RICRCICGRRICRCICGR |
| 13 | GICRCICGRGICRCICGR |
| 14 | GICRCICGKGICRCICGR |
| 15 | G1CRCYCGRGICRCICGR |
| 16 | GICRCICGRGICRCYCGR |
| 17 | GYCRCICGRGICRCICGR |
| 18 | GICRCICGRGYCRCICGR |
| 19 | GICYCICGRGICRCICGR |
| 20 | GICICICGYGICRCICGR |
| 21 | GICICICGRGICYCICGR |
| 22 | GICICICGRGICYCICGR |
| 23 | RGCICRCIGRGCICRCIG |
| 24 | RGCICRCIGRGCICRCIG |
| 25 | GICRCICGRGICRCICGR |
| 26 | GICRCICGKGICRCYCGR |

Polypeptide A may be an alpha-defensin selected from the group consisting of human neutrophil protein 1 (HNP-1), HNP-2, HNP-3, HNP-4, Human defensin 5 and Human defensin 6, an analogue, or a fragment thereof. The alpha defensin may be from mice, monkeys, rats, rabbits, guinea pigs, hamster, horse, elephant, baboon, hedgehog, horse, chimpanzee, orangutan, macaque, marmoset and the like from any mammalian origin.

In another example, the polypeptide A may be a beta-defensin selected from the group consisting of DEFB 1, DEFB 4A, DEFB 4B, DEFB 103A, DEFB 103B, DEFB 104A, DEFB 104B, DEFB 105A, DEFB 105B, DEFB 106A, DEFB 106B, DEFB 107A, DEFB 107B, DEFB 108B, DEFB 108 P1-4, DEFB 109 P1, DEFB 109 P1B, DEFB 109 P2-3, DEFB 110, DEFB 112-119, DEFB 121-136 and the like from any mammalian origin.

Polypeptide A may be a big defensins originating from (i) Amphioxus—Branchiostoma florida and Branchiostoma belcheri; (ii) Horseshoecrab—Tachypleus tridentatus; (iii) Mussel—Mytilus galloprovincialis; (iv) Clam—Ruditapes philippinarum; and (v) Oyster—Crassostrea giga and the like from any arthropod origin.

Polypeptide B may be a Type 1 Ribosome Inactivating Protein selected from the group consisting of Ebulitins, Nigritins, Amarandins, Amaranthus antiviral/RIP, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparins, Agrostin, Dianthins, DAPs, Dianthus chinensis′, Lychnin, Petroglaucin, Petrograndin, Saponaria ocymoides RIP, Vacuolas saporin, Saporins, Vaccaria hispanica RIP, Benincasins, Hispin, Byrodin's, Colocins, Cucumis figarei RIP, Melonin, C. moschata RIP, Cucurmosin, Moschatins, Pepocin, Gynostemmin, Gynostemma pentaphyllum RIP, Gypsophilin, Lagenin, Luffaculin, Luffangulin, Luffin, MORs, Momordin II, Momorcharin's, Momorcochin, Momorcochin-S, Sechiumin, Momorgrosvin, Trichoanguin, Kirilowin, α-trichosanthin, TAP-29, Trichokirin, Trichomislin, Trichosanthin, Karasurin, Trichomaglin, Trichobakin, Crotin, Euserratin Antiviral Protein GAP-31, Gelonin, Hura crepitans RIP, Curcin, Jathropa curcas RIP, Mapalmin, Manutins, α-pisavin, Charibdin, Hyacinthus orientalis RIP, Musarmin, Iris hollandica RIP, Cleroendrum aculeatum RIP, CIPs), Crip-31, Bouganin, Bougainvilla spectbilis RIP, Bougainvillea×buttiana Antiviral protein 1 (BBAP1), Malic enzymes, MAP-S, pokeweed antiviral proteins (PAP), PD-SI, DP-S2, Dodecandrin, PIP, PIP2, Phytolacca octandra anti-viral proteins, Hordeum vulgare RIPs, Hordeum vulgare sub sp. Vulgare Translational inhibitor II, Secale cereale RIP, Tritin, Zea diploperemis RIPs, Malus×domestica RIP, Momordica Anti-HIV Protein, Gelonium multiflorum, Mirabilis expansa 1, phage MU1, betavulgin (Bvg), curcin 2, saporin 6, Maize RIP (B-32), Tobacco RIP (TRIP), Beetins, Mirabilis antiviral protein (MAP), Trichosanthin (TCS), luffins, Momorcharins, Ocymoidin, Bryodin, Pepopsin, 3-trichosanthin, Camphorin, YLP, Insularin, Barley RIP, Tritins, Lamjarin, Volvariella volvacea RIP and the like from any plant origin.

Polypeptide C may be selected from the group consisting of Cyclotides, Siamycins, NP-06, Gramicidin A, Circulins, Kalatas, Ginkbilobin, Alpha-Basrubin, Lunatusin, Sesquin, Tricyclon A, Cycloviolacins, Polyphemusins, hfl-B5, Protegrins (Pig Cathelicidin), Rat Defensins, Human 11-defensins, Temporins, Caerins, Ranatuerins, Reptile Defensin, Piscidin's, Lactoferricin B, Rabbit Neutrophils, Rabbit α-Defensin, Retrocyclins, Human α-Defensins, Human β-defensin III (HBD3), Rhesus minidefensin (RTD-1,θ-defensin), rhesus θ-defensins, Human neutrophil peptides, Cecropin As, Melittin, EP5-1, Magainin 2s, hybrid (CE-MA), hepcidin TH1-5, Epinecidin-1, Indolicidin, Cathelicidin-4, LL-37 Cathelicidin, Dermaseptins, Maximins, Brevinins, Ranatuerins, Esculentins, Maculatin 1.3, Maximin H5 and Piscidins, Mundticin KS Enterocin CRL-35, Lunatusin, FK-13 (GI-20 is a derivative), Tachyplesins, Alpha-MSH, Antiviral protein Y3, Palustrin-3AR, Ponericin L2, Spinigerin, Melectin, Clavanin B, Cow cathelicidin's, Guinea pig cathelicidin CAP11, Sakacin 5X, Plectasin, Fungal Defensin, GLK-19, lactoferrin (Lf) peptide 2, Alloferon 1, Uperin 3.6, Dahlein 5.6, Ascaphin-8, Human Histatin 5, Guineapig neutrophils, Mytilins, EP5-1, Hexapeptide (synthetic) Corticostatin IV Rabbit Neutrophil 2, Aureins, Latarcin, Plectasin, Cycloviolins, Vary Peptide E, Palicourein, VHL-1.

In particular, polypeptide C may be Gaegurin 5, Gaegurin 6, their analogues, derivatives or fragments thereof, which may have pro-apoptotic properties that may act upon drug sensitive and multidrug resistant tumour cell lines.

Polypeptide D may be bi-functional peptides i.e. 2-domain fusion molecules that act on 2 separate active sites. Polypeptide D may be pro-apoptotic peptide. In particular, polypeptide D may be a Bax-derived membrane-active peptide. Bax-derived membrane-active peptides are apoptosis-inducing peptides that may be capable of causing apoptosis in cancer cells. For example, polypeptide D may be (KLAKLAK)2, SSX2, D-K₄R₂L₉ (Hoskin D. W. et al, 2008), p18 (Tang C et al, 2010) and the like.

In particular, (KLAKLAK)2 may be conjugated with leukemia cell differentiating peptide motifs; with bcl-2 antisense oligonucleotides targeting mitochondrial outer membrane permeability; to αᵥβ₃ integrin receptors targeting endothelial cell apoptosis; to self-assembling cylindrical nanofibres targeting breast cancer cells and to CGKRK glioblastoma-homing peptide motifs together with (KLAKLAK)2 being coated on iron oxide ‘nanoworms’. More particularly, (KLAKLAK)2 may be conjugated with MAP30.

A Cationic Antimicrobial Peptide (CAP) may be an anti-microbial CAP that may have anticancer and/or antiviral properties. CAPs may be a maximum of 100 amino acids in length. CAPs may either be a naturally occurring CAP with sequence with reported anticancer properties or a synthetic CAP sequence with anticancer properties. CAPs may mostly be of animal origin. However, there may also be CAPs, which are from plants, which include but are not limited to cyclotides. For example, bacteria CAPs may include but are not limited to Siamycin, NP-06 and Gramicidin A. Plant CAPs may include Circulin A, B, Kalata B1 and B8; Plant CAPs which may function as entry inhibitors may include Kalata B8, Ginkbilobin, Alpha-Basrubin, Lunatusin and Sesquin, Circulin A, C and D, Tricyclon A and Cycloviolacin H4. Animal CAPs may include Polyphemusin I and II, hfl-B5, Protegrin (Pig Cathelicidin), Rat Defensin NP1, NP2, NP3 and NP4, Human β-defensin I and II, Temporin A, Temporin-LTc, Temporin-Pta, Caerin 1.1, Ranatuerin 6 and 9, Reptile Defensin and Piscidin 1 and 2, Lactoferricin B, Rabbit Neutrophil-1 Corticostatin III a, Rabbit Neutrophil-3A, Rabbit α-Defensin, Retrocyclin-1, Retrocyclin-2, Retrocyclin-3, Human α-Defensin HNP-1, 2, 3, 4, 5 & 6, Human β-defensin III (HBD3), Rhesus minidefensin (RTD-1,θ-defensin), RTD-2 rhesus θ-defensin, RTD-3 rhesus θ-defensin, Human neutrophil peptide-2, Human neutrophil peptide-3 and human neutrophil peptide-4, Cecropin A, Melittin, EP5-1, Magainin 2, hepcidin TH1-5, and Epinecidin-1, Indolicidin, Cathelicidin-4, Human neutrophil peptide-1, LL-37 Cathelicidin, Dermaseptin-S1, S4 and S9, Maximin 1, 3, 4 and 5, Brevinin 1, Ranatuerin 2P, 6 and 9 Esculentin 2P, Esculentin-1 Arb, Caerin 1.1, 1.9 and 4.1, Brevinin-2-related, Maculatin 1.3, Maximin H5 and Piscidin 1 and 2. Other CAPs may include Mundticin KS Enterocin CRL-35, Lunatusin, FK-13 (GI-20 is a derivative), Tachyplesin I, Alpha-MSH, Antiviral protein Y3, Piscidin 3, Palustrin-3AR, Ponericin L2, Spinigerin, Melectin, Clavanin B, Cow cathelicidin BMAP-27, BMAP-28, Guinea pig cathelicidin CAP11, Sakacin 5X, Plectasin, Fungal Defensin, GLK-19, lactoferrin (Lf) peptide 2, Kalata B8, Tricyclon A, Alloferon 1, Uperin 3.6, Dahlein 5.6, Ascaphin-8, Human Histatin 5, Guineapig neutrophil CAP2 & CAP1, Mytilin B & C, EP5-1, and Hexapeptide (synthetic) Corticostatin IV Rabbit Neutrophil 2.

Cationic antimicrobial peptides (CAP) may exhibit cytotoxic activity against cancer cells as the electrostatic attraction between negatively charged components of cancer cells are attracted to positively charged CAPs resulting first in binding and then further on in cell disruption. Cancer cells may carry a net negative charge due to over-expression of phosphatidylserine, 0-glycosylated mucins and heparin sulphate. Furthermore, cancer cells may have increased numbers of microvilli leading to an increase in cell surface area, which may in turn enhance their vulnerability to CAP action. CAPs are also known for various antiviral properties and some of them also possess anticancer properties.

The Type 1 RIP may:
(iv) act as a pro-apoptotic polypeptide which upregulate pro-apoptotic genes that may include but not limited to caspase-12, Bax and the like, or downregulate anti-apoptotic gene including but not limited to Bcl-2 and the like in tumour or cancer cells (Fan, J-M., et al, Mol Biotechnol, 2008, 39, 79-86);
(v) act as a DNA glycosylase/apurinic (AP) lyase capable of irreversibly relaxing tumour or cancer cell supercoiled DNA and catalyzing double-stranded breakage to form inactive products;
(vi) act in alternative cytochrome patways as well as Mn²⁺ and Zn²⁺ interactions with negatively charged surfaces next to catalytic sites, facilitating DNA substrate binding instead of directly participating in catalysis (Wang et al, Cell, 1999, 99, 433-442);
(vii) as an RNA N-Glycosidase which hydrolyses the N-C glycosidic bond of adenosine at position 4324 of the universally conserved sarcin/ricin domain (S/R domain) of the 28S-rRNA in the eukaryotic ribosome and render it incapable of carrying out protein synthesis thus, functionally, blocking translation.

In particular, the type 1 RIP may be selected from the group consisting of α-Ebulitin, β-Ebulitin, γ-Ebulitin, Nigritin f1, Nigritin f2, Amarandin-S, Amaranthus antiviral/RIP, Amarandin-1, Amarandin-2, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparin 1, Asparin 2, Agrostin, Dianthin 29, DAP-30, DAP-32, Dianthin 30, Dianthus chinensis RIP1, Dianthus chinensis RIP2, Dianthus chinensis RIP3, Lychnin, Petroglaucin, Petrograndin, Saponaria ocymoides RIP, Vacuolas saporin, Saporin-1, Saporin-2, Saporin-3, Saporin-5, Saporin-6, Saporin-7, Saporin-9, Vaccaria hispanica RIP, Benincasin, α-benincasin, ρ-benincasin, Hispin, Byrodin I, Byrodin II, Colocin I, Colocin 2, Cucumis figarei RIP, Melonin, C. moschata RIP, Cucurmosin, Moschatin, Moschatin I, Moschatin II, Moschatin III, Moschatin IV, Moschatin V, Pepocin, Gynostemmin I, Gynostemmin II, Gynostemmin III, Gynostemmin IV, Gynostemmin V, Gynostemma pentaphyllum RIP, Gypsophilin, Lagenin, Luffaculin, Luffangulin, Luffin-alpha, Luffin-B, MOR-I, MOR-II, Momordin II, Alpha-momorcharin, β-momorcharin, γ δ-momorcharin, γ-momorcharin, Momorcochin, Momorcochin-S, Sechiumin, Momorgrosvin, Trichoanguin, α-kirilowin, β-kirilowin, α-trichosanthin, TAP-29, Trichokirin, Trichomislin, Trichosanthin, Karasurin-A, Karasurin-B, Trichomaglin, Trichobakin, Crotin 2, Crotin 3, Euserratin 1, Euserratin 2, Antiviral Protein GAP-31, Gelonin, Hura crepitans RIP, Curcin, Jathropa curcas RIP, Mapalmin, Manutin 1, Manutin 2, α-pisavin, Charibdin, Hyacinthus orientalis RIP, Musarmin 1, Musarmin 2, Musarmin 3, Musarmin 4, Iris hollandica RIP, Cleroendrum aculeatum RIP, CIP-29, CIP-34, Crip-31, Bouganin, Bougainvilla spectbilis RIP, Bougainvillea×buttiana Antiviral protein 1 (BBAP1), malic enzyme 1 (ME1), ME2, MAP-S, pokeweed antiviral protein (PAPa-1), PAPa-2, PAP-alpha, PAP-I, PAP-II, PAP-S, PD-SI, DP-S2, Dodecandrin, Anti-viral protein PAP, PIP, PIP2, Phytolacca octandra anti-viral protein, Phytolacca, octandra anti-viral protein II, Hordeum vulgare RIP-I, Hordeum vulgare RIP-II, Hordeum vulgare sub sp. Vulgare Translational inhibitor II, Secale cereale RIP, Tritin, Zea, diploperemis RIP-I, Zea diploperemis RIP-II, Malus×domestica RIP, Momordica Anti-HIV Protein (MAP30), Gelonium multiflorum (GAP31), pokeweed antiviral protein (PAP), Mirabilis expansa 1 (ME1), malic enzyme 2 (ME2), Bougainvillea×buttiana antiviral protein 1 (BBAP1), phage MU1, betavulgin (Bvg), curcin 2, saporin 6, Maize RIP (B-32), Tobacco RIP (TRIP), beetin (BE), BE27, Mirabilis antiviral protein (MAP), Trichosanthin (TCS), α-luffin, α-Momorcharin (α-MMC), β-MMC luffin, Ocymoidin, Bryodin, Pepopsin, f3-trichosanthin, Camphorin, YLP, Insularin, Barley RIP, Tritins, Lamjarin, Volvariella volvacea RIP, and the like from any plant origin.

MAP30 polypeptide or Ribosomal Inactivating Protein may act in a pro-apoptotic manner to destroy tumour or cancer cells selectively. In particular, MAP30 polypeptide may be selectively pro-apoptotic to Non-Hodgkin's Lymphoma cells. The anti-HIV and antitumor peptides and truncated polypeptides of MAP30 are disclosed in U.S. Pat. No. 6,652,861. Table 4 in U.S. Pat. No. 6,652,861 lists the various MAP30 fragments and those with either a positive or negative antitumor effect. In particular, Type 1 Ribosomal Inhibiting Proteins (RIP) especially MAP30, are known to have robust and broad-spectrum anticancer activity against a range of cancer cell types.

In particular, polypeptide A may be a Retrocyclin, polypeptide B may be MAP30 and polypeptide C may be a Dermaseptin. More in particular, polypeptide A may be Retrocyclin 101 (RC101) and polypeptide C may be Dermaseptin 1. A polypeptide comprising RC101, MAP30 and Dermaseptin 1 as polypeptide A, B and C respectively is termed RetroMAD1 in the present invention.

In particular, polypeptide A may comprise amino acid sequence with SEQ ID NO: 4, a fragment or variant thereof, polypeptide B may comprise amino acid sequence with SEQ ID NO:5, a fragment or variant thereof, and polypeptide C may comprise amino acid sequence with SEQ ID NO:6, a fragment or variant thereof.

More in particular, the fusion protein according to any aspect of the present invention may comprise the amino acid sequence SEQ ID NO:1. The fusion protein or the basic unit of the fusion protein may have a molecular weight of about 10-50 kDa. In particular, the molecular weight of the fusion protein may be 36.5, 37, 37.5, 37.8, 38, 39, 40, 41, 41.2, 43 or 48 kDa. The fusion protein may comprise repeats of the basic unit. A skilled person would understand that the weight of the fusion protein would be dependent on the multiples of the basic unit present in the protein. The nucleic acid coding for the fusion protein of SEQ ID NO:1 may be found in SEQ ID NO:2. The sequences are provided in Table 1 b below.

In particular, polypeptide B may be Type 1 RIP, or a fragment thereof, and polypeptide C may be Cationic AntiMicrobial Peptide, or a fragment thereof; and - may be a direct linkage or a linker peptide.

In particular, the fusion protein may comprise the formula XIV:

C-B-C

wherein, polypeptide C is CAP, an analogue, or a fragment thereof, polypeptide B is Type 1 RIP, or a fragment thereof, and - may be a direct linkage or a linker peptide.

In particular, the fusion protein may comprise the formula XX or XXI:

B-D or D-B

Respectively, wherein, polypeptide B is MAP30, an analogue, or a fragment thereof, polypeptide D is a synthetic anticancer sequence (KLAKLAK)2, or a fragment thereof, and - may be a direct linkage or a linker peptide.

TABLE 1b
Sequences of polypeptides and polynucleotides of the present invention.

| SEQ | |
|---|---|
| ID | |
| NO. | Sequences |
| 1 | MKYLLPTAAAGLLLLAAQPAMAMGRICRCICGRGICRCICGVPGVGVPGVGGATGSDVNFDLSTATAKTY |
| | TKFIEDFRATLPFSHKVYDIPLLYSTISDSRRFILLDLTSYAYETISVAIDVTNVYVVAYRTRDVSYFFK |
| | ESPPEAYNILFKGTRKITLPYTGNYENLQTAAHKIRENIDLGLPALSSAITTLFYYNAQSAPSALLVLIQ |
| | TTAEAARFKYIERHVAKYVATNFKPNLAIISLENQWSALSKQIFLAQNQGGKFRNPVDLIKPTGERFQVT |
| | NVDSDVVKGNIKLLLNSRASTADENFITTMTLLGESVVEFPWALWKTMLKELGTMALHAGKAALGAAADT |
| | ISQGTQVPGVGVPGVGKLAAALEHHHHHH |
| 2 | atgaaatacctgctgccgaccgctgctgctggtctgctgctcctcgctgcccagccggcgatggccatgg |
| | ggcgtatttgccgttgcatttgcggccgtggcatttgccgctgcatctgtggcgtgccgggtgttggtgt |
| | tccgggtgtgggtggtgcgaccggatccgatgtgaactttgatctgagcaccgcgaccgcgaaaacctat |
| | accaaattcatcgaagattttcgtgcgaccctgccgtttagccataaagtgtatgatatcccgctgctgt |
| | atagcaccattagcgatagccgtcgttttattctgctggatctgaccagctatgcgtatgaaaccattag |
| | cgtggcgattgatgtgaccaacgtgtatgtggtggcgtatcgtacccgtgatgtgagctactttttcaaa |
| | gaaagcccgccggaagcgtacaacattctgtttaaaggcacccgtaaaattaccctgccgtataccggca |
| | actatgaaaacctgcagaccgcggcgcataaaattcgtgaaaacatcgatctgggcctgccggccctgag |
| | cagcgcgattaccaccctgttttattataacgcgcagagcgcgccgagcgcgctgctggtgctgattcag |
| | accaccgcggaagcggcgcgttttaaatatattgaacgccacgtggcgaaatatgtggcgaccaacttta |
| | aaccgaacctggccattattagcctggaaaaccagtggagcgccctgagcaaacaaatttttctggccca |
| | gaaccagggcggcaaatttcgtaatccggtggatctgattaaaccgaccggcgaacgttttcaggtgacc |
| | aatgtggatagcgatgtggtgaaaggcaacattaaactgctgctgaacagccgtgcgagcaccgcggatg |
| | aaaactttattaccaccatgaccctgctgggcgaaagcgtggtggaattcccgtgggcgctgtggaaaac |
| | catgctgaaagaactgggcaccatggcgctgcatgcgggtaaagcggcgctgggtgcggcagcggatacc |
| | attagccagggcacccaggttccgggcgtgggcgttccgggcgttggtaagcttgcggccgcactcgagc |
| | accaccaccaccaccactga |
| 3 | [VPXVG]ₙ |
| 4 | GRICRCICGRGICRCICG |
| 5 | GSDVNFDLSTATAKTYTKFIEDFRATLPFSHKVYDIPLLYSTISDSRRFILLDLTSYAYETISVAIDVTN |
| | VYVVAYRTRDVSYFFKESPPEAYNILFKGTRKITLPYTGNYENLQTAAHKIRENIDLGLPALSSAITTLF |
| | YYNAQSAPSALLVLIQTTAEAARFKYIERHVAKYVATNEKPNLAIISLENQWSALSKQIFLAQNQGGKFR |
| | NPVDLIKPTGERFQVTNVDSDVVKGNIKLLLNSRASTADENFITTMTLLGESVVEFPW |
| 6 | ALWKTMLKELGTMALHAGKAALGAAADTISQGTQ |

Modifications and changes may be made in the structure of the peptides of the present invention and DNA segments which encode them and still obtain a functional molecule that encodes a protein or peptide with desirable characteristics. The amino acids changes may be achieved by changing the codons of the DNA sequence. For example, certain amino acids may be substituted for other amino acids in a protein structure without appreciable loss of interactive binding capacity with structures such as, for example, microorganism-binding regions of fusion proteins. Since it is the interactive capacity and nature of a protein that defines that protein's biological functional activity, certain amino acid sequence substitutions can be made in a protein sequence, and, of course, it's underlying DNA coding sequence, and nevertheless obtains a protein with like properties. Various changes may be made in the peptide sequences of the disclosed compositions, or corresponding DNA sequences, which encode said proteins without appreciable loss of their biological utility or activity. Amino acid substitutions of the fusion protein according to the present invention may be possible without affecting the antimicrobial effect of the isolated peptides of the invention, provided that the substitutions provide amino acids having sufficiently similar properties to the ones in the original sequences.

Examples of polypeptides according to any aspect of the present invention may be found in Table 1c and the DNA and protein sequences may be found in Tables 1d and 1e respectively.

TABLE 1c
Examples of fusion peptides

| Example | Polypeptide A | Polypeptide B | Polypeptide C | Sequencing listing |
|---|---|---|---|---|
| Fusion peptide | Defensin | RIP | CAP | |
| RetroMAD1 | Retrocyclin 101 | MAP30 | Dermaseptin1 | SEQ ID NO: 1 |
| RetroGAD1 | Retrocyclin 101 | GAP31 | Dermaseptin1 | SEQ ID NO: 36 |
| Tamapal1 | Tachyplesin | MAP30 | Alloferon1 | SEQ ID NO: 34 |
| Amatalin | AVBD103 | MAP30 | Mytillin C10C | SEQ ID NO: 28 |
| Example | Polypeptide C | Polypeptide B | Polypeptide D | Sequencing listing |
| Fusion peptide | CAP | RIP | Pro-apoptotic | — |
| | | | peptide | |
| K5 | Gaegurin 5 | MAP30 | (KLAKLAK)2 | SEQ ID NO: 35 |

TABLE 1d
DNA sequences of Amatilin, RetroGAD1, Tampal1 and K5

| | SEQ | |
|---|---|---|
| Fusion | ID | |
| Protein | NO. | DNA Sequence |
| Amatilin | 37 | GGGCAGTGAGCGGAAGGCCCATGAGGCCAGTTAATTAAGAGGTACCGAATTCTCAT |
| | | TCGGTTTGTGTAGATTGAGAAGAGGTTTCTGTGCTCACGGTAGATGTAGATTCCCA |
| | | TCCATCCCAATCGGTAGATGTTCCAGATTCGTTCAGTGTTGTAGAAGAGTTTGGGT |
| | | CCCAGGTGTTGGTGTTCCAGGTGTTGGAGGTGCTACTGGTTCTGATGTTAACTTCG |
| | | ACTTGTCCACTGCTACTGCTAAGACTTACACTAAGTTCATCGAGGACTTCAGAGCT |
| | | ACTTTGCCATTCTCCCACAAGGTTTACGACATCCCTTTGTTGTACTCCACTATCTC |
| | | CGACTCCAGAAGATTCATCTTGTTGAACTTGACTTCCTACGCTTACGAGACTATCT |
| | | CCGTTGCTATCGACGTTACAAACGTTTACGTTGTTGCTTACAGAACTAGAGATGTT |
| | | TCCTACTTCTTCAAAGAGTCCCCACCAGAGGCTTACAACATCTTGTTCAAGGGTAC |
| | | TAGAAAGATCACTTTGCCATACACTGGTAACTACGAGAACTTGCAGACTGCTGCTC |
| | | ACAAGATCAGAGAGAACATCGACTTGGGTTTGCCAGCTTTGTCCTCCGCTATCACT |
| | | ACTTTGTTCTACTACAACGCTCAGTCCGCTCCATCCGCTTTGTTGGTTTTGATCCA |
| | | GACTACTGCTGAGGCTGCTAGATTCAAGTACATCGAGAGACACGTTGCTAAGTACG |
| | | TTGCTACAAACTTCAAGCCAAACTTGGCTATCATCTCCTTGGAGAACCAGTGGTCT |
| | | GCTTTGTCCAAGCAGATCTTCTTGGCTCAAAACCAGGGTGGTAAGTTCAGAAACCC |
| | | AGTCGACTTGATCAAGCCAACCGGTGAGAGATTCCAGGTTACTAATGTTGACTCCG |
| | | ACGTTGTTAAGGGTAACATCAAGTTGTTGTTGAACTCCAGAGCTTCCACTGCTGAC |
| | | GAGAACTTCATCACTACTATGACTTTGTTGGGTGAGTCCGTTGTTAACTCCTGTGC |
| | | TTCCAGATGTAAGGGTCACTGTAGAGCTAGAAGATGTGGTTACTACGTTTCCGTTC |
| | | TGTACAGAGGTAGATGTTACTGTAAATGTTTGAGATGTGTCCCCGGTGTTGGAGTC |
| | | CCTGGTGTCGGTGCGGCCGCGAGCTCATGGCGCGCCTAGGCCTTGACGGCCTTCCG |
| | | CCAATTCGC |
| RetroGAD1 | 38 | CGAATTGGCGGAAGGCCGTCAAGGCCACGTGTCTTGTCCAGGTACCGAATTCGGAA |
| | | TCTGTAGATGCATCTGCGGTAGAGGTATCTGCAGATGTATTTGTGGAAGAGTCCCA |
| | | GGTGTTGGTGTTCCAGGTGTTGGAGGTGCTACTGGTTCTGGTTTGGACACTGTTTC |
| | | ATTCTCCACTAAGGGTGCTACTTACATCACTTACGTTAACTTTTTGAACGAGTTGA |
| | | GAGTTAAGTTGAAGCCAGAGGGTAACTCCCACGGTATCCCTTTGTTGAGAAAGAAG |
| | | TGTGACGACCCAGGTAAGTGTTTCGTTTTGGTTGCTTTGTCCAACGACAACGGTCA |
| | | GTTGGCTGAGATTGCTATCGACGTTACTTCCGTTTACGTTGTTGGTTACCAGGTTA |
| | | GAAACAGATCCTACTTCTTCAAGGACGCTCCAGACGCTGCTTACGAAGGTTTGTTC |
| | | AAGAACACTATCAAGACTAGATTGCACTTCGGTGGTTCCTACCCATCTTTGGAAGG |
| | | TGAGAAGGCTTACAGAGAGACTACTGACTTGGGTATCGAGCCATTGAGAATCGGTA |
| | | TCAAGAAGTTGGACGAGAACGCTATCGACAACTACAAGCCAACTGAGATCGCTTCC |
| | | TCCTTGTTGGTTGTTATCCAGATGGTTTCCGAGGCTGCTAGATTCACTTTCATCGA |
| | | GAACCAGATCAGAAACAACTTCCAGCAGAGAATCAGACCAGCTAACAACACTATTT |
| | | CCTTGGAGAACAAGTGGGGTAAGTTGTCCTTCCAGATCAGAACATCCGGTGCTAAC |
| | | GGTATGTTCTCTGAGGCTGTTGAGTTGGAGAGAGCTAACGGTAAGAAGTACTACGT |
| | | TACTGCTGTTGACCAGGTTAAGCCAAAGATCGCTTTGTTGAAGTTCGTTGACAAGG |
| | | ACCCAAAGGGTTTGTGGTCCAAGATCAAAGAGGCTGCTAAGGCTGCTGGTAAGGCT |
| | | GCTTTGAATGCTGTTACTGGTTTGGTTAACCAGGGTGACCAACCATCTGTCCCTGG |
| | | TGTTGGAGTCCCTGGTGTCGGTGCGGCCGCGAGCTCTGGAGCACAAGACTGGCCTC |
| | | ATGGGCCTTCCGCTCACTGC |
| Tampal1 | 39 | GGATCCGTTCCGGGTGTGGGTGTTCCGGGTGTTGGTAAATGGTGTTTCGTGTTTGT |
| | | TATCGCGGTATTTGTTATCGTCGTTGTCGTGTGCCAGGCGTTGGCGTTCCAGGCGT |
| | | GGGTGGTGCAACCGGTAGTGATGTTAATTTTGATCTGAGCACCGCAACCGCAAAAA |
| | | CCTATACCAAATTTATCGAAGATTTTCGTGCAACCCTGCCGTTTAGCCATAAAGTT |
| | | TATGATATTCCGCTGCTGTATAGCACCATTAGCGATAGCCGTCGTTTTATTCTGCT |
| | | GAATCTGACCAGCTATGCCTATGAAACCATTAGCGTTGCAATTGATGTGACCAATG |
| | | TTTATGTTGTTGCATATCGTACCCGTGATGTGAGCTATTTTTTCAAAGAAAGCCCT |
| | | CCGGAAGCCTATAACATTCTGTTTAAAGGCACCCGCAAAATCACCCTGCCGTATAC |
| | | CGGTAATTATGAAAATCTGCAGACCGCAGCACATAAAATTCGCGAAAATATTGATC |
| | | TGGGTCTGCCTGCACTGAGCAGCGCAATTACCACCCTGTTTTATTACAATGCACAG |
| | | AGCGCACCGAGCGCACTGCTGGTTCTGATTCAGACCACCGCAGAAGCAGCACGCTT |
| | | TAAATACATTGAACGTCATGTTGCCAAATACGTGGCCACCAACTTTAAACCGAATC |
| | | TGGCAATTATTAGCCTGGAAAATCAGTGGTCAGCACTGAGCAAACAAATTTTTCTG |
| | | GCACAGAATCAGGGTGGCAAATTTCGTAATCCGGTTGATCTGATTAAACCG |
| | | ACCGGTGAACGTTTTCAGGTTACCAATGTTGATAGTGATGTGGTGAAAGGCAACAT |
| | | TAAACTGCTGCTGAATAGCCGTGCAAGCACCGCAGATGAAAACTTTATTACCACCA |
| | | TGACCCTGCTGGGTGAAAGCGTTGTTAATGTTCCTGGTGTTGGCGTGCCTGGTGTT |
| | | GGTCATGGTGTTAGCGGTCATGGTCAGCATGGTGTTCATGGTTAAAAGCTT |
| K5 | 40 | GGATCCGTTCCGGGTGTGGGTGTTCCGGGTGTTGGCTTTCTGGGTGCACTGTTTAA |
| | | AGTTGCAAGCAAAGTTCTGCCGAGCGTTAAATGTGCAATTACCAAAAAATGTGTTC |
| | | CTGGCGTTGGTGTTCCAGGCGTGGGTGGTGCAACCGGTAGTGATGTTAATTTTGAT |
| | | CTGAGCACCGCAACCGCAAAAACCTATACCAAATTTATCGAAGATTTTCGTGCAAC |
| | | CCTGCCGTTTAGCCATAAAGTTTATGATATTCCGCTGCTGTATAGCACCATTAGCG |
| | | ATAGCCGTCGTTTTATTCTGCTGAATCTGACCAGCTATGCCTATGAAACCATTAGC |
| | | GTTGCAATTGATGTGACCAATGTTTATGTTGTTGCATATCGTACCCGTGATGTGAG |
| | | CTATTTTTTCAAAGAAAGCCCTCCGGAAGCCTATAACATTCTGTTTAAAGGCACCC |
| | | GCAAAATCACCCTGCCGTATACCGGTAATTATGAAAATCTGCAGACCGCAGCACAT |
| | | AAAATTCGCGAAAATATTGATCTGGGTCTGCCTGCACTGAGCAGCGCAATTACCAC |
| | | CCTGTTTTATTACAATGCACAGAGCGCACCGAGCGCACTGCTGGTTCTGATTCAGA |
| | | CCACCGCAGAAGCAGCACGCTTTAAATACATTGAACGTCATGTTGCCAAATACGTG |
| | | GCCACCAACTTTAAACCGAATCTGGCAATTATTAGCCTGGAAAATCAGTGGTCAGC |
| | | ACTGAGCAAACAAATTTTTCTGGCACAGAATCAGGGTGGCAAATTTCGTAATCCGG |
| | | TTGATCTGATTAAACCGACCGGTGAACGTTTTCAGGTTACCAATGTTGATAGTGAT |
| | | GTGGTGAAAGGCAACATTAAACTGCTGCTGAATAGCCGTGCAAGCACCGCAGATGA |
| | | AAACTTTATTACCACCATGACCCTGCTGGGTGAAAGCGTTGTTAATGTTCCAGGTG |
| | | TTGGTGTGCCTGGTGTGGGTAAACTGGCAAAACTGGCCAAAAAACTGGCTAAGCTG |
| | | GCGAAATAAAAGCTT |

TABLE 1e
Polypeptide sequences of Amatilin, RetroGAD1, Tampal1 and K5

| | SEQ | |
|---|---|---|
| Fusion | ID | |
| Protein | NO. | Protein Sequence |
| Amatilin | 28 | SFGLCRLRRGFCAHGRCRFPSIPIGRCSRFVQCCRRVWVPGVGVPGVGGATGSDVNF |
| | | DLSTATAKTYTKFIEDFRATLPFSHKVYDIPLLYSTISDSRRFILLNLTSYAYETIS |
| | | VAIDVTNVYVVAYRTRDVSYFFKESPPEAYNILFKGTRKITLPYTGNYENLQTAAHK |
| | | IRENIDLGLPALSSAITTLFYYNAQSAPSALLVLIQTTAEAARFKYIERHVAKYVAT |
| | | NFKPNLAIISLENQWSALSKQIFLAQNQGGKFRNPVDLIKPTGERFQVTNVDSDVVK |
| | | GNIKLLLNSRASTADENFITTMTLLGESVVNSCASRCKGHCRARRCGYYVSVLYRGR |
| | | CYCKCLRCVPGVGVPGVG |
| RetroGAD | 136 | GICRCIGRGICRCICGRVPGVGVPGVGGATGSGLDTVSFSTKGATYITYVNFLNELR |
| | | VKLKPEGNSHGIPLLRKKCDDPGKCFVLVALSNDNGQLAETATDVTSVYVVGYQVRN |
| | | RSYFFKDAPDAAYEGLFKNTIKTRLHFGGSYPSLEGEKAYRETTDLGIEPLRIGIKK |
| | | LDENAIDNYKPTEIASSLLVVIQMVSEAARFTFIENQIRNNFQQRIRPANNTISLEN |
| | | KWGKLSFQIRTSGANGMFSEAVELERANGKKYYVTAVDQVKPKIALLKFVDKDPKGL |
| | | WSKIKEAAKAAGKAALNAVTGLVNQGDQPSVPGVGVPGVG |
| Tampal1 | 34 | VPGVGVPGVGKWCFRVCYRGICYRRCRVPGVGVPGVGGATGSDVNFDLSTATAKTYT |
| | | KFIEDFRATLPFSHKVYDIPLLYSTISDSRRFILLNLTSYAYETISVAIDVTNVYVV |
| | | AYRTRDVSYFFKESPPEAYNILFKGTRKITLPYTGNYENLQTAAHKIRENIDLGLPA |
| | | LSSAITTLFYYNAQSAPSALLVLIQTTAEAARFKYIERHVAKYVATNFKPNLAIISL |
| | | ENQWSALSKQIFLAQNQGGKFRNPVDLIKPTGERFQVTNVDSDVVKGNIKLLLNSRA |
| | | STADENFITTMTLLGESVVNVPGVGVPGVGHGVSGHGQHGVHG |
| K5 | 35 | VPGVGVPGVGFLPLLAGLAANFLPTIICFISYKCVPGVGVPGVGGATGSDVNFDLS |
| | | TATAKTYTKFIEDFRATLPFSHKVYDIPLLYSTISDSRRFILLNLTSYAYETISVA |
| | | IDVTNVYVVAYRTRDVSYFFKESPPEAYNILFKGTRKITLPYTGNYENLQTAAHKI |
| | | RENTDLGLPALSSAITTLFYYNAQSAPSALLVLIQTTAEAARFKYIERHVAKYVAT |
| | | NFKPNLAIISLENQWSALSKQIFLAQNQGGKFRNPVDLIKPTGERFQVTNVDSDVV |
| | | KGNIKLLLNSRASTADENFITTMTLLGESVVNVPGVGVPGVGKLAKLAK |
| | | KLAKLAK |

K5 and Tamapal1 have been shown to be capable of close to 99% inhibition of PI3K at low concentrations of 5 μg/ml, Both these peptide drugs could be a potential medical drugs that function by inhibiting a Phosphoinositide 3-kinase enzyme which may be part of this pathway and therefore, through inhibition, often results in tumor suppression. This high level of inhibition of PI3K at such low drug concentrations may also be very useful in combinatorial anticancer drug regimes that may involve other drugs outside of this class or also with drugs within this class that work primarily on other pathways.

The fusion peptide according to any aspect of the present invention may be thermostable over a prolonged period of time withstanding action of digestive enzymes acting at their pH optima, being absorbed through the G.I. tract rapidly with a significant retention time, up-regulating and down-regulating cellular pathways in normal and virally infected cells, acting via oral delivery in various animal models including aquatic ones. Thermostability is an industrially significant attribute as cold-chain transportation will greatly increase logistics and handling costs that will contribute to the overall total cost of the medication. Also, if the drug is to be carried about to be consumed before meals, patient compliance will suffer if the requirement of low temperature storage in an absolute necessity. Thus, the ability to remain stable for 7 days even at elevated temperatures will allow for a wider usage and application of the therapeutic protein.

The medicament according to any aspect of the present invention may further comprise a pharmaceutically acceptable carrier, excipient, adjuvant, diluent and/or detergent. Such formulations therefore include, in addition to the fusion protein, a physiologically acceptable carrier or diluent, possibly in admixture with one or more other agents such as other antibodies or drugs, such as an antibiotic. Suitable carriers include, but are not limited to, physiological saline, phosphate buffered saline, phosphate buffered saline glucose and buffered saline. Alternatively, the fusion protein may be lyophilized (freeze dried) and reconstituted for use when needed by the addition of an aqueous buffered solution as described above. Routes of administration are routinely parenteral, including intravenous, intramuscular, subcutaneous and intraperitoneal injection or oral delivery. The best mode of administration may be systemic and/or local.

In particular, the medicament according to any aspect of the present invention may be suitable for oral administration as the medicament may have a high resistance to pepsin & trypsin proteolysis. In particular, the presence of MAP30 surprisingly renders the fusion protein according to any aspect of the present invention stable for oral administration.

The medicament may further comprise a detergent. The detergent may be selected from the group consisting of sodium-ursodeoxycholate, sodium glycylursodeoxycholate, potassium-ursodeoxycholate, potassium glycylursodeoxycholate, ferrous-ursodeoxycholate, ferrous glycylursodeoxycholate, ammonium-ursodeoxycholate, ammonium glycylursodeoxycholate, sodium-tauroursodeoxycholate, sodium-N-methylglycylursodeoxycholate, potassium-tauroursodeoxycholate, potassium-N-methyglycylursodeoxy-cholate, ferrous-tauroursodeoxycholate, ferrous-N-methyglycylursodeoxycholate, ammonium-tauroursodeoxycholate, ammonium-N-methyglycylursodeoxycholate, sodium-N-methyltauroursodeoxycholate, potassium-N-methyltauroursodeoxycholate, ferrous-N-methyltauroursodeoxycholate, ammonium-N-methyltauroursodeoxycholate, sodium-cholate, sodium-deoxycholate, potassium-cholate, potassium-deoxycholate, ferrous-cholate, ferrous-deoxycholate, ammonium-cholate, ammonium-deoxycholate, sodium-chenodeoxycholate, sodium-glycylcholate, potassium-chenodeoxycholate, potassium-glycylcholate, ferrous-chenodeoxycholate, ferrous-glycylcholate, ammonium-chenodeoxycholate, ammonium-glycylcholate, sodium-taurocholate, sodium-N-methylglycylcholate, potassium-taurocholate, potassium-N-methylglycylcholate, ferrous-taurocholate, ferrous-N-methylglycylcholate, ammonium-taurocholate, ammonium-N-methylglycylcholate, sodium-N-methyltaurocholate, sodium-glycyldeoxycholate, potassium-N-methyltaurocholate, potassium-glycyldeoxycholate, ferrous-N-methyltaurocholate, ferrous-glycyldeoxycholate, ammonium-N-methyltaurocholate, ammonium-glycyldeoxycholate, sodium-taurodeoxycholate, sodium-N-methylglycyldeoxycholate, potassium-taurodeoxycholate, potassium-N-methylglycyldeoxycholate, ferrous-taurodeoxycholate, ferrous-N-methyl glycyldeoxycholate, ammonium-taurodeoxycholate, ammonium-N-methylglycyldeoxycholate, sodium-N-methyltaurodeoxycholate, sodium-N-methylglycylchenodeoxycholate, potassium-N-methyltaurodeoxycholate, potassium-N-methylglycylchenodeoxycholate, ferrous-N-methyltaurodeoxycholate, ferrous-N-methylglycylchenodeoxycholate, ammonium-N-methyltaurodeoxycholate, ammonium-N-methylglycylchenodeoxycholate, sodium-N-methyltaurochenodeoxycholate, potassium-N-methyltaurochenodeoxycholate, ferrous-N-methyltaurochenodeoxycholate, ammonium-N-methyltaurochenodeoxycholate, ethyl esters of ursodeoxycholate, propyl esters of ursodeoxycholate, sodium-glycylchenodeoxycholate, potassium-glycylchenodeoxycholate, ferrous-glycylchenodeoxycholate, ammonium-glycylchenodeoxycholate, sodium-taurochenodeoxycholate, potassium-taurochenodeoxycholate, ferrous-taurochenodeoxycholate, ammonium-taurochenodeoxycholate, sodium deoxycholate and the like. In particular, the detergent may be sodium deoxycholate that allows for oral administration as it may result in the fusion protein not being digested in the gastrointestinal tract when consumed. This is a convenient mode of administration.

The detergent may be present at a concentration of 0.003-5% by weight. In particular, the concentration may be 0.01-4.5 wt %, 0.05-4 wt %, 0.1-3.5 wt %, 0.5-2 wt %, 1-1.5 wt %, and the like. In particular, the concentration of the detergent may be about 0.05 wt %.

The dosage of the ligand according to the present invention to be administered to a patient having tumour or cancer may vary with the precise nature of the condition being treated and the recipient of the treatment. The dose will generally be in the range of about 0.005 to about 1000 mg for an adult patient, usually administered daily for a period between 1 day to 2 years. In particular, the daily dose may be 0.5 to 100 mg per day. In particular the daily dose may be about 0.8, 1, 1.2, 1.5, 2, 2.5, 3.2, 4, 4.5, 5, 10, 15, 20, 30, 45, 50, 75, 80, 90, 95 mg per day. The dosage may be applied in such a manner that the ligand may be present in the medicament in concentrations that provide in vivo concentrations of said ligand in a patient to be treated of between 0.001 mg/kg/day and 5 mg/kg/day. In one embodiment, the medicament, the peptide or ligand according to the invention is present in an amount to achieve a concentration in vivo of 1 μg/ml or above with a maximum concentration of 100 μg/ml. the dosage regime may be varied depending on the results on the patient.

In one example, the patient may be given at least one medicament comprising at least a first fusion protein for a period of 1 month to 2 years. The first fusion protein may be taken for a period of 1, 2, 3, 4, 5, 6, 7, 8, 9, 10, 11, 12, 13, 14, 15 months. Once the first fusion protein appears less effective or not as effective as before on treating the cancer and/or tumour, a second fusion protein according to any aspect of the present invention may be administered to the patient. The second fusion protein may be different from the first fusion protein. Once the second fusion protein appears less effective or not as effective as before on treating the cancer and/or tumour, a third, fourth fifth, sixth etc. fusion protein according to any aspect of the present invention may be administered to the patient each protein may be different from the earlier protein. This dosage regime may prevent resistant cancer cells from proliferating thus providing an effective and efficient cancer therapy.

The medicament of the present invention can further contain at least one host defence molecule, such as lysozyme, lactoferrin and/or Reverse-Transcriptase inhibitor.

The medicament of the present invention may be a free flowing micronized powder comprising crystals around 0.5μ-1.5μ in size for use in tablet or capsule making.

The fusion protein and medicament according to any aspect of the present invention may have a broad spectrum of antimicrobial and/or anti cancer properties. In particular, the fusion protein and medicament may be useful in developing a broad spectrum, oral delivery antimicrobial and/or anticancer therapeutic. This may be especially beneficial to the many livestock industries which are under pressure from the threat of viral epizootics. This is particularly important as when world population rises, there is also more pressure on food production to become more productive.

The fusion protein according to any aspect of the present invention may be capable of maintaining its form in the digestive tract without fragmentation or enzymatic digestion. In one example, the fusion protein may be in a liquid form. In particular, the fusion protein may be ingested, as a drink diluted with water, or the like, and the retention time in either stomach or duodenum is only a matter of minutes allowing the protein to reach its target point without being digested.

The fusion protein and medicament according to any aspect of the present invention may be used for treatment and/or prevention of cancer. The cancer may be a microbe induced cancer. Microbes which induce cancer may include by are not limited to bacteria, viruses and the like. These microbes may be classified as Class A, B or C microbes. Class A microbes induce cancers including lymphomas by targeting immunocytes leading to immunosuppression. This immunosuppression also contributes to the cancer-inducing effects of class B microbes, which include local effects on parenchymal cells and induction of host responses. Class B microbes may induce the most commonly recognized microbe-associated cancers. Class C microbes are a postulated class in which a microbe produces local effects on epithelial tissues that change the regulation of a systemic operator (e.g., a hormone) that promotes cancer at a distant site. Non-limiting examples of class A agents include human T-cell lymphotrophic virus type 1, which may promote adult T-cell leukemia/lymphoma, and HIV, which may promote lymphoma development and, through immunosuppression, other microbe-induced malignancies including human herpesvirus-8 induced Kaposi's sarcoma and HPV-induced anogenital cancers as well as HSV1/2-induced anogenital or oral cancers.

The numerous examples of class B processes include carcinomas due to the hepatitis viruses, H. pylori and the like. Class C agents, with local effects that can lead to either distant or other local effects may include H. pylori—induced development of atrophic gastritis which could lead to repopulation with microbiota that are toxic to gastric tissue and directly oncogenic, or microbiome-induced disturbances in hormonal regulation could lead to cancers distant from the locus of the change.

In particular, cancer bacteria may include Salmonella typhi which may be associated with gallbladder cancer, Streptococcus bovis which may be associated with colorectal cancer, Chlamydia pneumoniae which may be associated with lung cancer, Mycoplasma which may be associated with formation of different types of cancer, Helicobacter pylori which may be linked to stomach cancer, gastric cancer, MALT lymphoma, esophageal cancer and the like.

Cancer viruses may be known as oncoviruses that may include DNA viruses and/or RNA viruses. The DNA viruses may include but are not limited by Human papilloma virus (HPV) which may cause transformation in cells through interfering with tumor suppressor proteins such as p53 and thus causing cancers such as cancers of cervix, anus, penis, vulva/vagina, and some cancers of the head and neck. Other DNA viruses include Kaposi's sarcoma-associated herpesvirus (KSHV or HHV-8) which may be associated with Kaposi's sarcoma, a type of skin cancer, Epstein-Barr virus (EBV or HHV-4) which may be associated with Burkitt's lymphoma, Hodgkin's lymphoma, post-transplantation lymphoproliferative disease, Nasopharyngeal carcinoma and the like, Merkel cell polyomavirus—a polyoma virus—may be associated with the development of Merkel cell carcinoma, Human cytomegalovirus (CMV or HHV-5) which may be associated with mucoepidermoid carcinoma and possibly other malignancies and the like.

RNA viruses include but are not limited to hepatitis A, B and C viruses which are associated with Hepatocellular carcinoma (liver cancer), human T-lymphotropic virus (HTLV-1) which is associated with Tropical spastic paraparesis and adult T-cell leukemia and the like.

The cancer may be selected from the group consisting of Non-Hodgkin's Lymphoma, brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, pontine glioblastoma, Kaposi's sarcoma, and any other type of solid or liquid cancer.

The fusion protein may be pegylated to aid in the medicament being suitable for oral delivery. In particular, the fusion protein may be pegylated with any PEG known in the art. The PEG may be selected from the group consisting of but not limited to PEG200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 3000, 3250, 3350, 3500, 3750, 4000, 4250, 4500, 4750, 5000, 5500, 6000, 6500, 7000, 7500, 8000 and the like.

In one aspect of the present invention there is provided a method of treating and/or preventing a microbial infection and/or cancer in a subject in need thereof, comprising administering to the subject an effective amount of the fusion protein orally before food intake.

In yet another aspect of the present invention there is provided the fusion protein or the medicament according to any aspect of the present invention for use in treating and/or preventing a microbial infection and/or cancer in a subject in need thereof, the fusion protein for oral administration and before food intake.

In particular, the microbial infection may be a viral infection. The vertebrate may be a mammal, fish or bird. Even more in particular, the mammal may be a non-human animal, non-aquatic animal and the like. In one example, if the animal is a shrimp, the fusion protein or medicament may be fed to the shrimp for at least 1 week. In particular, if the animal being treated is a head-on shrimp, treatment of the fusion protein and/or medicament according to any aspect of the present invention may be prescribed for at least 2 weeks. In another example, if the animal being treated is a headless shrimp, treatment of the fusion protein and/or medicament according to any aspect of the present invention may be prescribed for at least 1 week.

In one aspect of the present invention there is provided a method of improving oral delivery of at least one peptide to a subject, the method comprising the step of linking the peptide to a MAP30 protein.

The linker used may be any linker known in the art. In particular, the linker may be at SEQ ID NO:3 or SEQ ID NO:27. The peptide may be any peptide that results in the prevention, cure, or mitigation of a disease in any animal. All vaccines are intended to be included in this definition of pharmaceutically active agents. The peptide may by any peptide with therapeutic activity. In particular, the peptide may have antimicrobial and/or anticancer activity. More in particular, the peptide may have the structure of peptide X-MAP30-peptide X/Y, wherein X or Y may be any peptide known to have a therapeutic effect. The presence of MAP30 surprisingly renders any fusion protein stable for oral administration.

The fusion protein according to any aspect of the present invention, allows for a method of producing at least one heat stable naked protein drug that may be capable of retaining bioactivity at harsh temperatures of 60-70° C. for at least 15 minutes. In fact, the fusion protein of the present invention may be capable of retaining bioactivity at harsh temperatures of 60-70° C. for at least 15 minutes and further followed by exposure to 50-55° C. for a further 45 minutes.

The fusion protein according to any aspect of the present invention may also be capable of being fed to an aquatic animal along with its feed without major loss due to leaching into the water. There is thus provided a method of feeding the fusion protein of the present invention to an aquatic animal along with the feed without major loss of the protein via leaching into the water. In particular, a maximum retention of the fusion protein was found to be 8 hours in the muscle and 6 days in the hepatopancreas in shrimp.

A person skilled in the art will appreciate that the present invention may be practised without undue experimentation according to the method given herein. The methods, techniques and chemicals are as described in the references given or from protocols in standard biotechnology and molecular biology text books.

The fusion protein and/or pharmaceutical composition according to any aspect of the present invention may result in no or substantially no toxic side effects when taken by the subject.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Green, Molecular Cloning: A Laboratory Manual, Cold Springs Harbor Laboratory (Fourth Edition), New York (2012).

### Example 1

### Construction and Design of Expression Vector

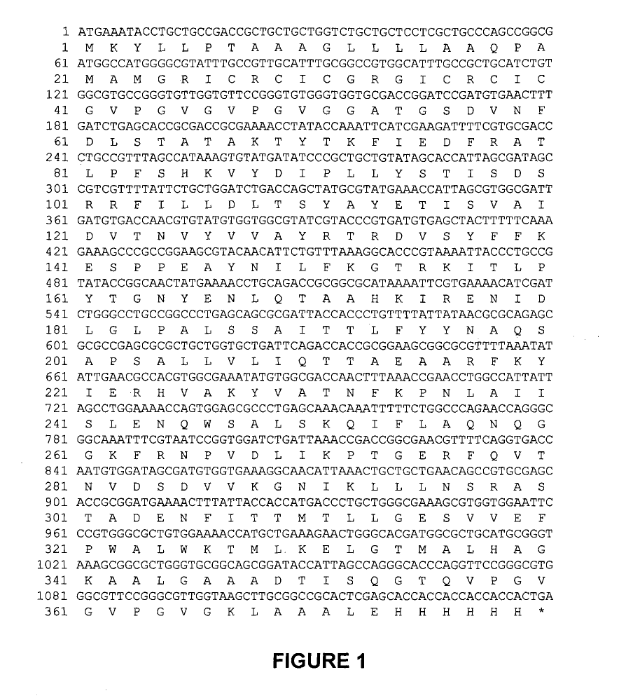

The gene encoding RetroMAD1 A-B-C with SEQ ID NO:1 was synthesized and cloned into backbone of vector pGA4 at the KpnI/SacI site by contract service (GeneArt AG, Germany). The expected product size was 1140 bp, which encoded a 379 amino acid and an expected size of 41.2 kDa. The polynucleotide sequence and the translated polypeptide sequence are shown in FIG. 1 from PCT. The gene was sub-cloned into a pET expression vector (Novagen), pET-26(b) at the NcoI/HindIII sites. Kanamycin was used as a marker for selection and maintenance of culture purposes. This vector was inducible under the addition of isopropyl-beta-D-thiogalactopyranoside (IPTG). The plasmid, pRMD1 was then transformed into BL21(DE23) cells (Novagen) and plated on a selective media with Kanamycin.

Expression of RetroMAD1 from E. coli

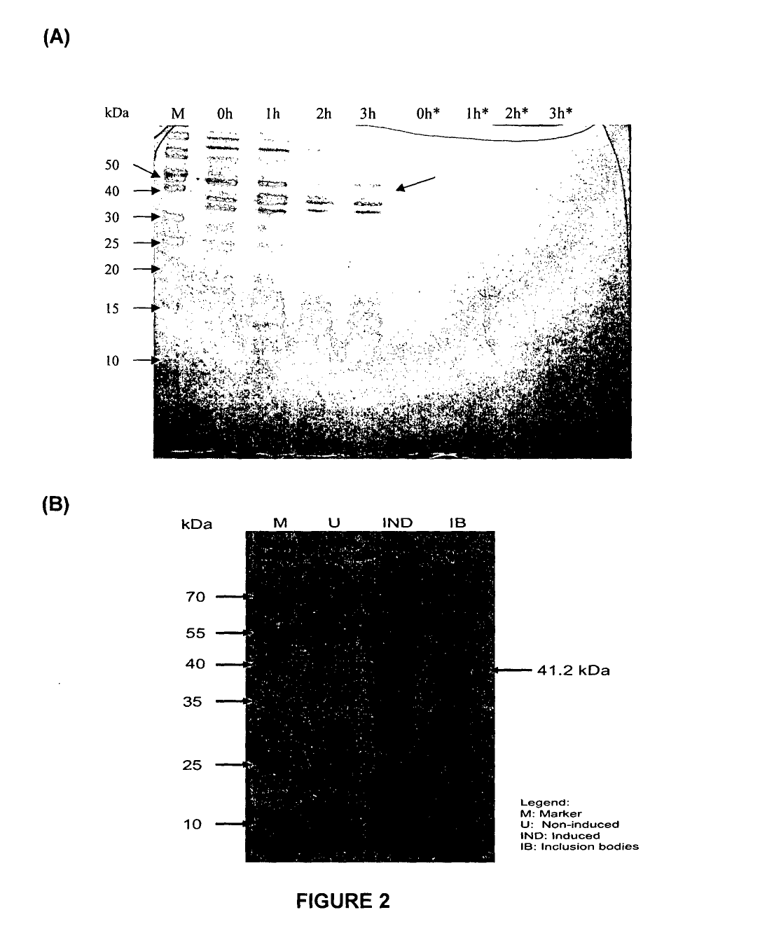

One recombinant clone was grown in 10 ml of LB Bertani (DIFCO) medium, supplemented with 30 μg/ml kanamycin, at 37° C. overnight. This culture was used to inoculate 100 ml of LB Bertani supplemented with 30 μg/ml kanamycin and grown at 37° C. until the optical reading was 0.4-0.6 at 600 nm. IPTG was added at 1.0 mM final concentration. The growth period continued for 3 hours. An SDS-PAGE analysis of the fraction of RetroMAD1 in cells extracted in electrophoresis loading buffer showed that a protein had a molecular mass of about 37.5 kDa, the expected molecular size of RetroMAD1 was produced in the induced cells only (FIG. 2A). Further solubility analysis by SDS-PAGE revealed that RetroMAD1 was found in the pellet fraction and not in the supernatant fraction of the E. coli indicating that the protein was expressed and produced as inclusion bodies as shown in FIG. 2B.

### Isolation and Purification of RetroMAD1

Cells from 100 ml of induced culture were harvested by centrifugation for 10 min at 5000×g at 15° C. The cells were suspended in a lysis buffer containing 20 mM Tris-HCl (pH 7.5), 10 mM EDTA and 1% Triton-X 100. Cells were disrupted by sonication. The insoluble fraction was isolated from the soluble fraction by centrifugation at 8,000×g for 20 min. The supernatant was discarded and the pellet was further washed by repeating the same step. The pellet was further washed twice with RO water by resuspension via sonication and separation by centrifugation.

### Solubilization of RetroMAD1

The insoluble material was dissolved and sonicated in 10 ml of 5-8 Urea or 6M Guanidine Hydrochloride and supplemented with 2-5% of Sodium-lauryl sarcosine and 100 mM (β-mercaptoethanol. The solubilisation was carried out overnight. The solubilised protein was separated from the bacterial cell wall by centrifugation at 8,000×g for 20 minutes.

### Refolding of RetroMAD1

Renaturation of the protein was carried out by using dialysis. The protein (10 ml) was dialysed in a 15 kDa molecular weight cut-off dialysis membrane (Spectra/Por Lab). The protein was dialysed in 5 L of RO water with the pH of 11.0 adjusted by NaOH. Incubation was done at room temperature for 15-20 hours. The refolded protein was transferred to a 50 ml tube and centrifuged at 8,000×g to separate any insoluble material. Renatured protein was stored at −20° C. until further use. The bioactivity of RetroMAD1 in the following examples is proof of successful refolding of the protein.

### Example 2

### Evidence of Bioavailability

The pharmacokinetic data of RetroMAD1 was derived in 6-8 weeks female ICR mice. Mice (48) were administered with single dose of RetroMAD1 of 70 ul per mouse which is a 50× dose of 0.2 mg/kg body weight given orally for ten days. Each day blood samples were drawn from the heart of three mice and one control. For the first day after the feed, the blood was collected after 30 min, 1 hour, 2 hour, 4 hour, 8 hour and 12 hours after oral administration and for the following days (up to day 10) the blood was collected just 30 min after administration. Each time point consisted of 3 mice fed orally with the drug and one control given PBS. Plasma concentration of RetroMAD1 was determined using an in house developed ELISA.

ELISA for Detecting RetroMAD1 in Mice Sera: In House Capture ELISA with Anti Human-IgG-HRP

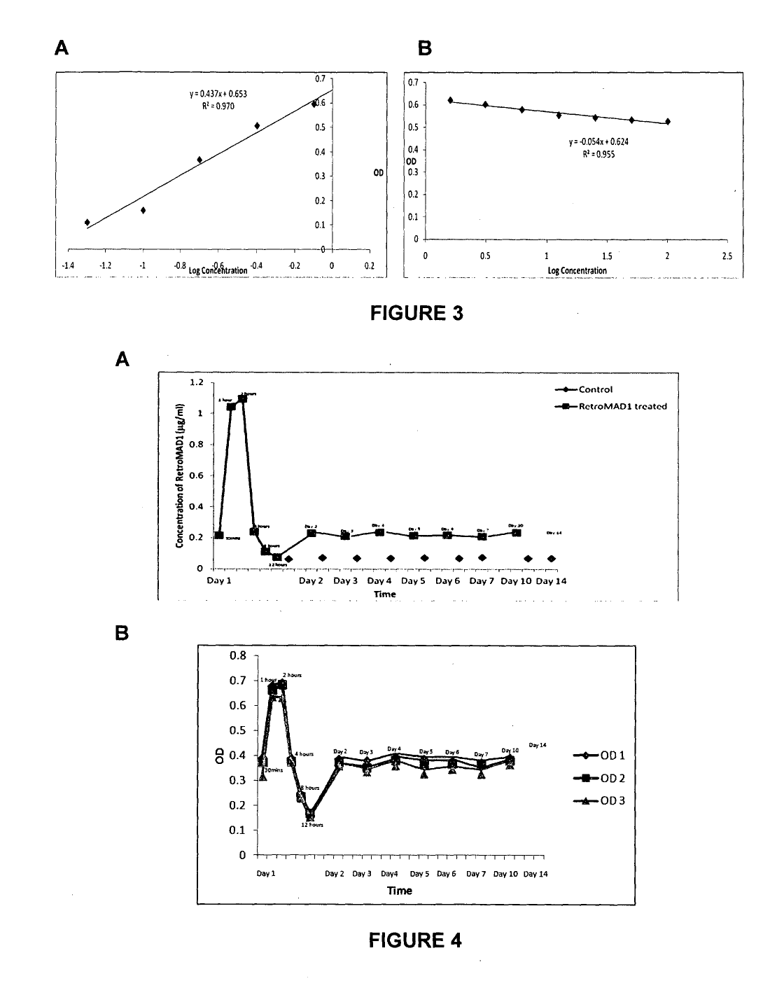

To prepare the capture antibody a cat was fed daily with RetroMAD1 and after 6 months blood harvested and serum extracted. This serum was used as the capture antibody. 100 ul/well of this polyclonal cat anti-RetroMAD1 antibody diluted 1:80 in coating buffer (0.2 M sodium carbonate-bicarbonate, ph 9.6) was adsorbed onto 96-well polystyrene ELISA plates. The plates were incubated at 4° C. overnight. Plates were washed three times with 0.05% Tween-20 in PBS 1×. 100 ul/well of mice serum diluted 1:2 in 0.05% BSA in PBS and were added to the wells. After incubation at 37 C for 1 h, plates were washed similarly and 100 ul of anti RetroMAD1 positive human serum diluted 1:2000 in 0.05% BSA in PBS, was added. This antibody was obtained from the Department of Medical Microbiology, Faculty of Medicine, University Malaya, Malaysia. After incubation at 37° C. for 1 h, plates were washed and 100 ul/well Rabbit anti-human IgG HRP conjugate diluted 1:6000 in 0.05% BSA in PBS, was added. After incubation at 37° C. for 1 h in the dark, plates were washed and 100 ul/well of OPD added to each well. Plates were incubated in the dark for 30 min at room temperature and reaction stopped with 50 ul/well of 4N H2SO4. Optical densities (OD) were measured at 490 nm and 600 nm as background. All OD readings were then converted to Log values to obtain concentrations in ug/ml and the standard curves provided in FIG. 3. The results of the tests are provided in Table 2 and FIGS. 4A and B. The PK/PD data showed that RetroMAD1 was detected in the serum as early as 30 min post feeding at about 0.2 μg/ml that reached a maximum at 1-2 hrs at 1-1.1 μg/ml before falling again to about 0.2 μg/ml at 4 hrs. By 12 hrs post feeding, levels were almost similar to the unfed controls indicating that the protein had been completely metabolized. Subsequent daily sampling 30 min post feeding indicated levels around 0.2 μg/ml. These data suggest bioavailability of the drug.

TABLE 2
Results of bioavailability test

| Day | Time | | OD 1 | OD 2 | OD 3 | Average | y = 0.437x + 0.6533 | |
|---|---|---|---|---|---|---|---|---|
| Day | 30 | mins | 0.391743 | 0.374396 | 0.317144 | 0.361094333 | −0.668662853 | 0.214455479 |
| 1 | 1 | hr | 0.683215 | 0.66296 | 0.637182 | 0.661119 | 0.017892449 | 1.042059336 |
| | 2 | hr | 0.632854 | 0.685153 | 0.692951 | 0.670319333 | 0.038945843 | 1.093819957 |
| | 4 | hr | 0.375195 | 0.376294 | 0.391285 | 0.380924667 | −0.623284516 | 0.238075927 |
| | 8 | hr | 0.234143 | 0.247498 | 0.229154 | 0.236931667 | −0.952787948 | 0.111483874 |
| | 12 | hr | 0.16735 | 0.154429 | 0.16771 | 0.163163 | −1.121594966 | 0.075579677 |
| | Control | | 0.132178 | | | 0.132178 | −1.192498856 | 0.064194991 |
| Day | 30 | mins | 0.387735 | 0.359613 | 0.372947 | 0.373431667 | −0.640430969 | 0.228859546 |
| 2 | Control | | 0.152749 | | | 0.152749 | −1.145425629 | 0.07154419 |
| Day | 30 | mins | 0.334864 | 0.352838 | 0.382846 | 0.356849333 | −0.678376812 | 0.209711955 |
| 3 | Control | | 0.149021 | | | 0.149021 | −1.153956522 | 0.070152553 |
| Day | 30 | mins | 0.360735 | 0.382153 | 0.395173 | 0.379353667 | −0.626879481 | 0.236113337 |
| 4 | Control | | 0.148574 | | | 0.148574 | −1.154979405 | 0.069987518 |
| Day | 30 | mins | 0.386559 | 0.367518 | 0.327878 | 0.360651667 | −0.66967582 | 0.213955857 |
| 5 | Control | | 0.156574 | | | 0.156574 | −1.136672769 | 0.073000735 |
| Day | 30 | mins | 0.347217 | 0.369173 | 0.3797746 | 0.3653882 | −0.658837071 | 0.219362774 |

| | | | | | |
|---|---|---|---|---|---|
| 6 | Control | 0.14443 | 0.14443 | −1.164462243 | 0.068475901 |

### Example 3

### Further Evidence of Bioavailability

In Guinea Pig PK/PD study, prior to experiment with RetroMAD1, the Guinea Pigs were starved overnight. The guinea pigs were then fed orally with RetroMAD1 according to their body weight; guinea pigs weighing from 380-430 g were fed orally with 250 μl of 3.5 mg/ml RetroMAD1, while guinea pigs weighing from 440-520 g were fed with 300 μl of 3.5 mg/ml RetroMAD1, and the controls were fed with water. At each time point, 3 guinea pigs were fed orally with RetroMAD1 and 3 guinea pig as control were fed with water. Before bleeding, the guinea pigs were given anesthesia (Ketamine and Xylazine) intramuscularly; the sedative dose was calculated using the following formula,

Ketamine=(45×body weight of the guinea pig)/(Concentration of Ketamine, 100 mg/ml)

Xylazine=(4.5×body weight of the guinea pig)/(Concentration of Xylazine, 20 mg/ml)

The guinea pigs were bled at 0, 30 mins, 1, 4 and 6 hours after feeding, blood samples were drawn from the heart. Serum of both control (untreated) and RetroMAD1-treated mice was collected for capture ELISA assay to determine the concentration of RetroMAD1 in the blood system.

Guinea pig organs were harvested. The organs are stomach, small intestine, liver, kidney.

Organs

| | |
|---|---|
| Stomach, Small | Collected into 15 ml of PBS for capture ELISA assay |
| Intestine | |
| Kidney, Liver | Snap freeze with liquid nitrogen |
| Kidney, Liver | Collected into distilled water and homogenized |
| Kidney, Liver | Collected into formalin for histology study |

Capture ELISA

Capture ELISA using rabbit serum and anti-RetroMAD1 positive human serum was used to determined concentration of RetroMAD1 in the blood, stomach and small intestine.

In this capture ELISA, 100 μl of 1:1000 rabbit serum containing polyclonal rabbit anti-RetroMAD1 antibody was coated onto each well. The plates were incubated at 4° C. overnight. Plates were washed six times with 0.05% Tween-20 in PBS. The plates were then blocked with blocking buffer (10% BSA in PBS), 200 μl of blocking buffer was added to each well and was incubated for 2 hours at 37° C. Plates were then washed six times with 0.05% Tween-20 in PBS. 100 μl of guinea pig sample (serum/small intestine supernatant/stomach supernatant) were added to each wells and incubated at 37° C. for 1 hour, plates were then washed. 100 ul of 1:2500 anti-RetroMAD1 positive human serum. After incubation at 37° C. for 1 hour, the plates were washed. 100 μl 1:4800 Rabbit anti-human IgG HRP was added and incubated at 37° C. for 1 hour in the dark, plates were then washed. 100 ul of OPD added to each well and the plates were incubated in the dark for 30 min at room temperature. Finally, 50 ul of 4N H2SO4 was added to each well to stop the reaction. Optical densities (OD) were measured at 490 nm and 600 nm as background.

A standard curve was first generated by doing the capture ELISA as described above with RetroMAD1 of 1/2 dilution, the concentrations of RetroMAD1 are 100, 50, 25, 12.5, 6.25, 3.125, 1.6, 0.8, 0.4, 0.2 and 0.1 μg/ml. The equation of the standard curve was used to determine concentration of RetroMAD1 in serum, stomach and small intestine.

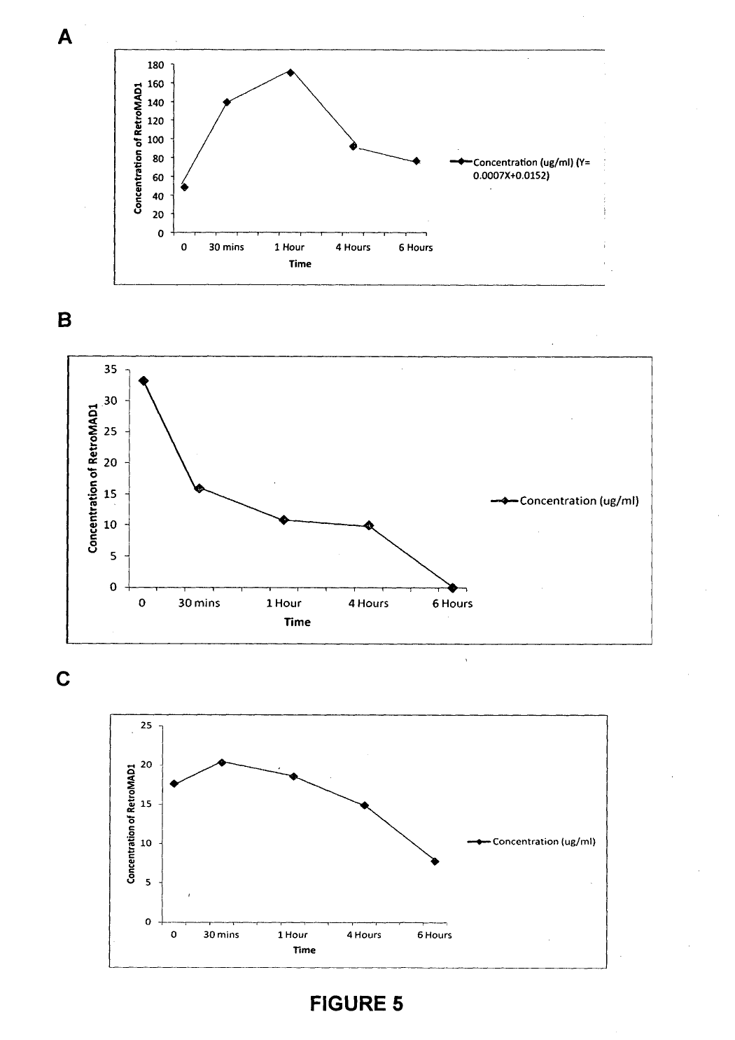

The PK/PD data for guinea pig serum is shown in Table 3 and FIG. 5A, result showed that RetroMAD1 was detected in the serum as early as 30 min post feeding at about 130 μg/ml that reached a maximum at 1 hour at 170 μg/ml before falling again to about 90 μg/ml at 4 hours and 76 μg/ml at 6 hours. At 6 hours, the concentration of RetroMAD1 is more than the unfed controls indicating that the protein is not fully metabolized yet.

Data for guinea pig small intestine supernatant is shown in Table 4 and FIG. 5B. Result showed that highest concentration of RetroMAD1 was detected at 30 minutes at about 16 μg/ml. The concentration of RetroMAD1 then starts to fall to about 11 μg/ml at 1 hour, 9 μg/ml at 4 hours. And is then release from the small intestine at 6 hours where no RetroMAD1 was detected.

TABLE 3
Results of bioavailability test in serum of guinea pig

| | | | | | Concentration (ug/ml) |
|---|---|---|---|---|---|
| Time | OD1 | OD2 | OD3 | Average | (Y = 0.0007X + 0.0152) |
| 0 | 0.047875 | 0.048515 | 0.050432 | 0.048941 | 48.2 |
| 30 mins | 0.118283 | 0.103765 | 0.115757 | 0.112602 | 139.15 |
| Control | 0.042267 | 0.042888 | 0.031889 | 0.039015 | 34.02 |
| 1 Hour | 0.132425 | 0.138091 | 0.132801 | 0.134439 | 170.34 |
| Control | 0.033272 | 0.043224 | 0.0398 | 0.038765 | 33.66 |
| 4 Hours | 0.089203 | 0.066944 | 0.082124 | 0.079424 | 91.75 |
| Control | 0.034081 | 0.031897 | 0.037074 | 0.034351 | 27.36 |
| 6 Hours | 0.06819 | 0.06453 | 0.074069 | 0.06893 | 76.76 |
| Control | 0.034571 | 0.032915 | 0.026507 | 0.031331 | 23.04 |

TABLE 4
Results of bioavailability test in Supernatant (Small Intestine) of guinea pig

| | | | | | Concentration (ug/ml) |
|---|---|---|---|---|---|
| Time | OD1 | OD2 | OD3 | Average | (Y = 0.0007X + 0.0152) |
| 0 | 0.036135 | 0.04063 | 0.038485 | 0.038417 | 33.17 |
| 30 mins | 0.035252 | 0.021182 | 0.022579 | 0.026338 | 15.91 |
| Control | 0.020616 | 0.021508 | 0.017995 | 0.02004 | 6.91 |
| 1 Hour | 0.021445 | 0.02472 | 0.022229 | 0.022798 | 10.85 |
| Control | 0.024589 | 0.021682 | 0.025826 | 0.024032 | 12.62 |
| 4 Hours | 0.022667 | 0.024702 | 0.019184 | 0.022184 | 9.98 |
| Control | 0.023728 | 0.031897 | 0.019516 | 0.025047 | 14.07 |
| 6 Hours | 0.017626 | 0.007617 | 0.007499 | 0.010914 | −6.12 |
| Control | 0.031773 | 0.036568 | 0.026049 | 0.031463 | 23.23 |

Data for guinea pig stomach supernatant is shown in Table 5 and FIG. 5C. Results showed that concentration of RetroMAD1 is highest at 30 minutes after feeding; 20.33 μg/ml. Concentration of RetroMAD1 starts to fall after 30 minutes from 18.55 μg/ml at 1 hour to 14.86 μg/ml at 4 hours and 7.77 μg/ml at 6 hours.

TABLE 5
Results of bioavailability test in Supernatant (Stomach) of guinea pig

| | | | | | Concentration (ug/ml) |
|---|---|---|---|---|---|
| Time | OD1 | OD2 | OD3 | Average | (Y = 0.0007X + 0.0152) |
| 0 | 0.029778 | 0.026176 | 0.026629 | 0.027528 | 17.61 |
| 30 mins | 0.027148 | 0.029376 | 0.031765 | 0.02943 | 20.33 |
| Control | 0.019232 | 0.031765 | 0.023121 | 0.024706 | 13.58 |
| 1 Hour | 0.026634 | 0.020743 | 0.037239 | 0.028205 | 18.58 |
| Control | 0.029548 | 0.020057 | 0.020743 | 0.023449 | 11.78 |
| 4 Hours | 0.032441 | 0.023119 | 0.021245 | 0.025602 | 14.86 |
| Control | 0.026809 | 0.020738 | 0.018296 | 0.021948 | 9.64 |
| 6 Hours | 0.021402 | 0.023904 | 0.016614 | 0.02064 | 7.77 |
| Control | 0.023544 | 0.021402 | 0.024692 | 0.023213 | 11.45 |

### Example 4

### Thermostability Trials

Thermostability of a protein is a property of which the protein maintains its activity and stability at high temperatures (above 40° C.). The majority of proteins denature in high heat, strong acid/base disruptions, alcohol, strong reducing agent and heavy metal salt.

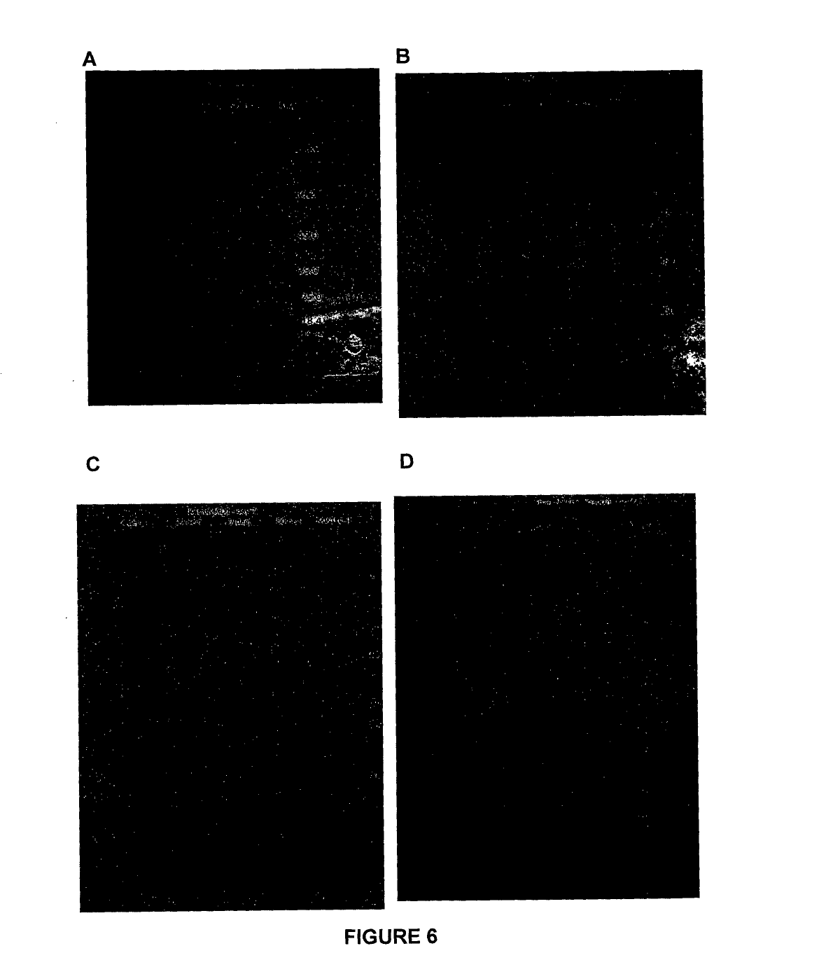

Protein stability under different temperatures was determined by keeping RetroMAD1 in multiple 1.5 ml Eppendorf tubes at 4° C. in a conventional refrigerator, 27° C.+/−1° C. in a laboratory which had 24 hour air-conditioning that maintained a narrow temperature range, in a conventional incubator oven set at 37° C. and in a laboratory oven set at 50° C. As RetroMAD1 is a protein of 41.2 kDa, running it on an SDS-PAGE gel and comparing the gel band of the sample stored at 4° C. with those kept at the other temperatures will reveal its stability. Up to day 7, the intensity of the gels remained the same irrespective of temperature up to 50 C. Up to day 30, the intensity was similar for the samples stored at 4° C., 27+/−1° C. and 37° C. Unfortunately, a sample for 50° C. was not kept for the 30^{th} day. Based on the results as shown in FIG. 6, RetroMAD1 is stable up to 50° C. for a week and 37° C. for a month.

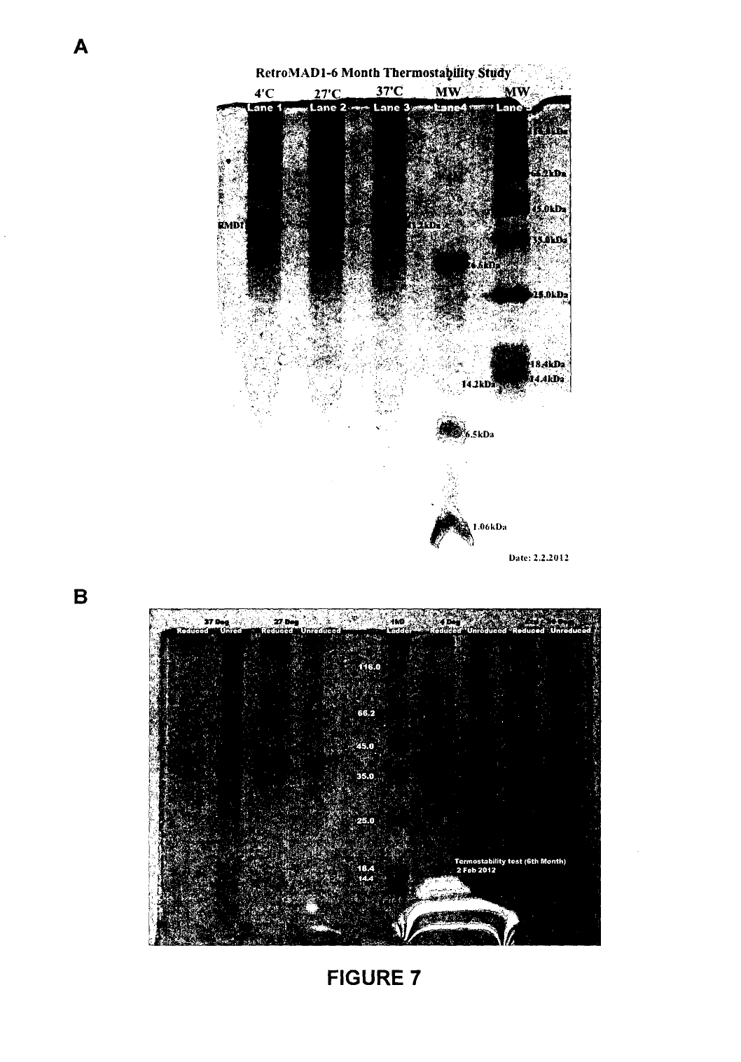

As shown in FIG. 7A, by using RetroMAD 1(RMD1) at 4° C. as a control, RMD 1 in 27° C. has overall similar amount and thickness of visible bands. There are no obvious or visible bands above 45.0 kDa for RMD1 in 37° C. compared to the control as well as RMD1 in 27° C.

Introducing a sample from −20° C. as a control actually to counter check the thermostability of sample from 4° C. which had been using throughout the experiment for 6 months duration showed clearly that the bands patterns on 27° C., 4° C. and −20° C. are similar while several cell debris bands were missing in 37° C. sample as shown in FIG. 7B. This confirms that RetroMAD1 can be stable up to 6 months.

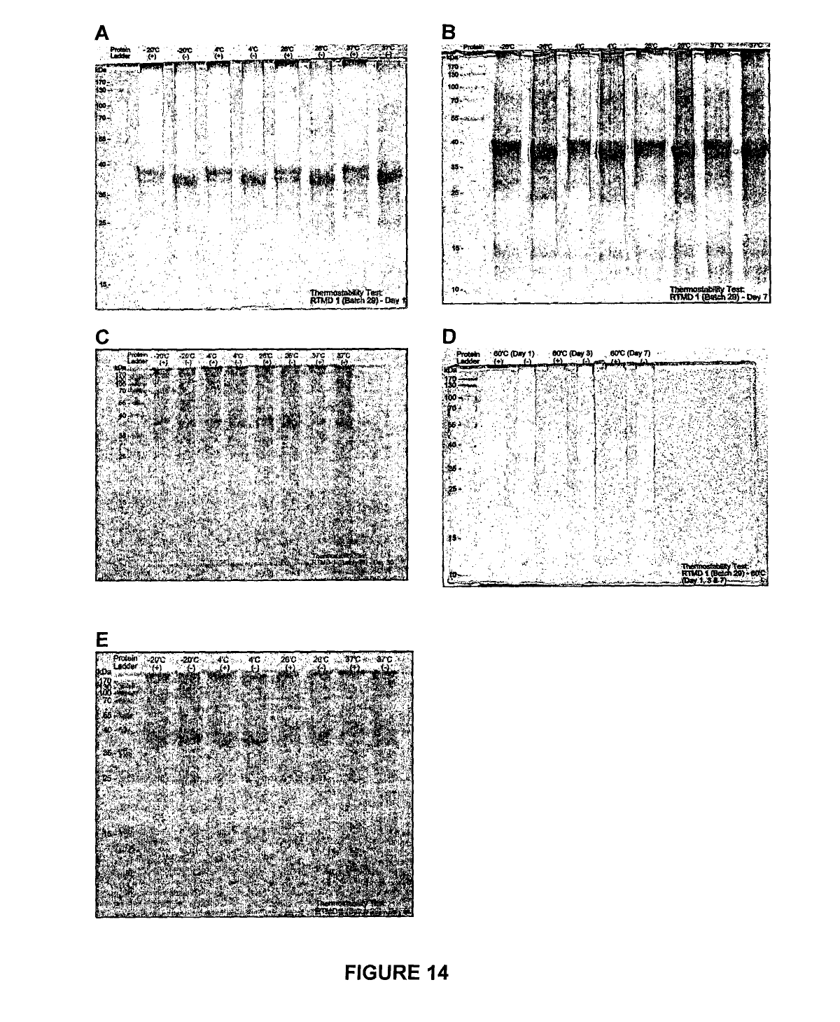

The experiment was repeated and the results shown in FIG. 14. The test for RetroMAD1 was carried out up to 90 days.

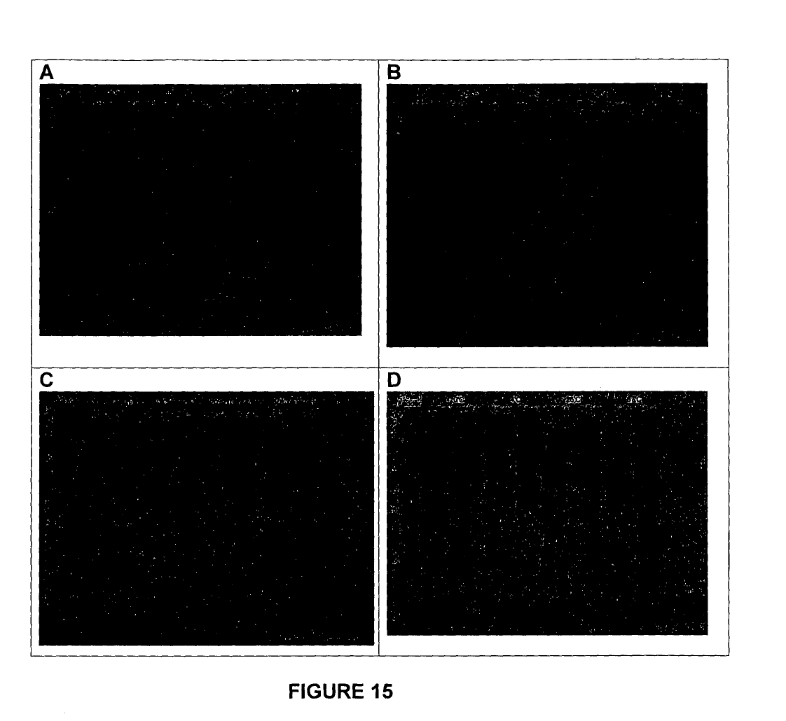

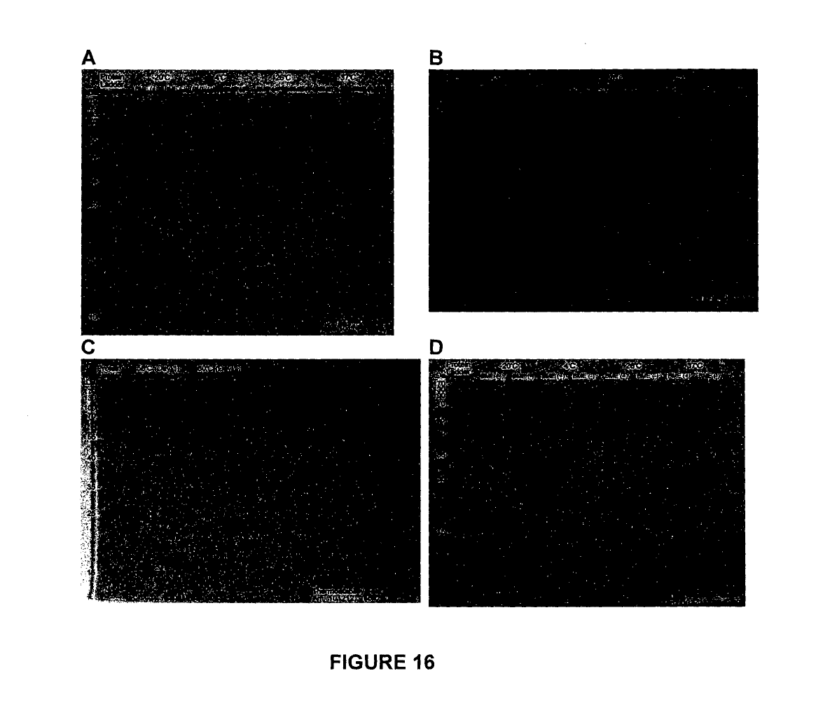

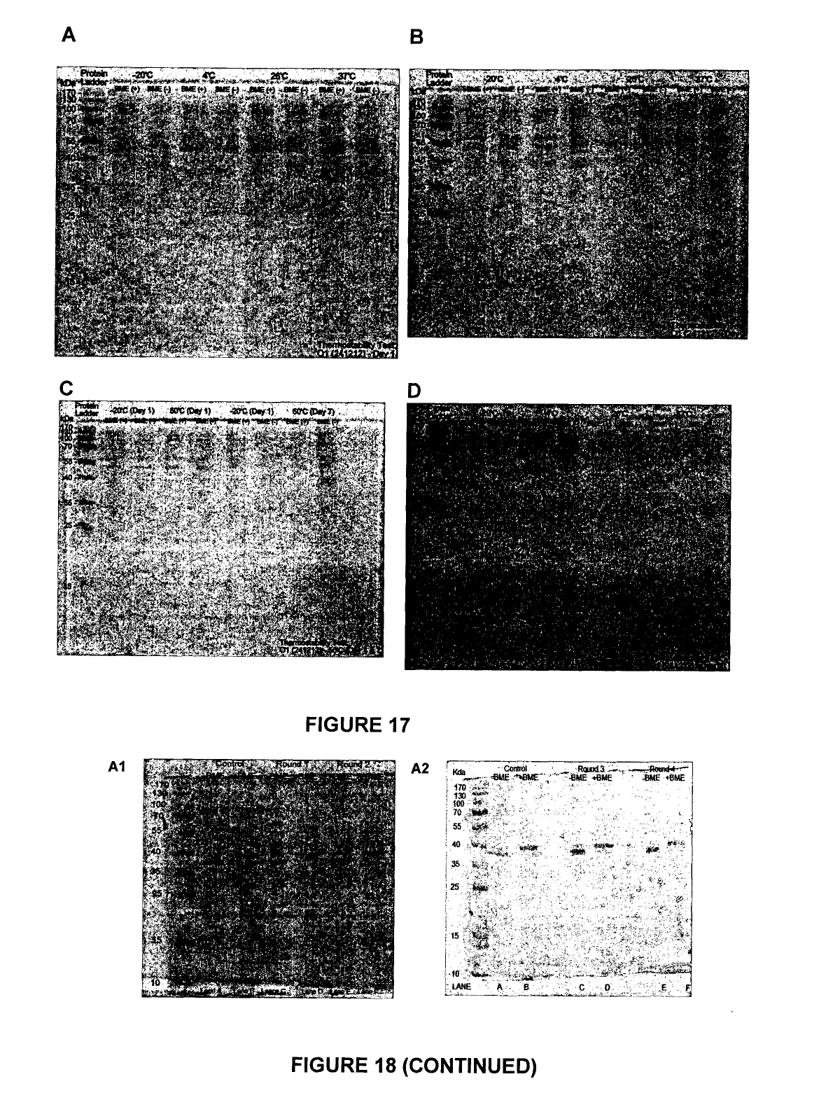

Other protein drugs (RetroGAD1, Amatilin and Tamapal1) were also incubated at −20° C., 4° C., 26° C., 37° C. and 50° C. for different time pints (1 day, 7 days and 30 days). The structural nature of protein drugs were then determined by SDS-page with the comparison to the control (protein drugs are incubated in −20° C.). The results are provided in FIGS. 15-17.

All the protein drugs i.e. RetroMAD1, RetroGAD1, Amatilin and Tamapal1, were intact despite the influence of temperature and the amount of time that the drugs were treated for. The bands and sizes for each protein drug were the same as the respective control sample which was maintained at −20° C.

### Example 5

### Ability to Withstand Proteolytic Digestion

The ability of RetroMAD1 to withstand action of digestive enzymes acting at their pH optima is shown in Table 6 below.

50 mM DTT was prepared amd added into pre-dissolved RetroMAD1 protein (1:1) made according to Example 1 and mixed. This was heated at 95° C. for 10 minutes and used to carry out enzyme assays with proteases such as Trypsin (pH8) (Lonza, Walkersville), α-Chymotrypsin (pH8) (Sigma-Aldrich) and Pepsin (pH2) (Sigma-Aldrich). After 10 minutes of heating at 95° C., the reaction was allowed to cool to room temperature (Approx. 10 mins) and proteases added to a final ratio of 1:100 (w/w) (protease:protein). This was incubated at 37° C. for 2 hours and protease activity terminated by incubating the mixture at 65° C. for 15 minutes. SDS-PAGE was used to analyze the fragments.

Other fusion proteins provided in Table 7 were made according to the method of Example 1 and the results of their fragmentation provided in Table 6.

TABLE 6
Results of fragmentation of fusion proteins according to the present inventION

| | Size of | SEQ ID | | No of bands after protease digestion | | |
|---|---|---|---|---|---|---|
| Drug | drug | NO: | Structure of drug | Pepsin | Trypsin | Chymot [symbol] psin |
| Amatalin | 40 kDa | 28 | A-B-C | No | No | No |
| (AM) | | | (AVBD103-MAP30-MYTILINC10C) | fragment | fragment | fragmen [symbol] |
| CT | 36 kDa | 29 | A-A-B-C | No | No | No |
| | | | (CERCROPIN A-CERCROPIN D- | fragment | fragment | fragmen [symbol] |
| | | | TAP29-DAP30-LATARCIN 2A) | | | |
| AB | 32 kDa | 30 | (RETROCYCLIN 101-MORMODICA | No | No | No |
| | | | ANTI-HIV PROTEIN 30) | fragment | fragment | fragmen [symbol] |
| BA | 32 kDa | 31 | (MORMODICA ANTI-HIV PROTEIN | No | No | No |
| | | | 30- RETROCYCLIN 101) | fragment | fragment | fragmen [symbol] |
| BC | 35 kDa | 32 | (MORMODICA ANTI-HIV PROTEIN | No | No | No |
| | | | 30- DERMASEPTIN 1) | fragment | fragment | fragmen [symbol] |
| CB | 35 kDa | 33 | DERMASEPTIN 1- MORMODICA | No | No | No |
| | | | ANTI-HIV PROTEIN 30 | fragment | fragment | fragmen [symbol] |
| Tamapal1 | 35.93 kDa | 34 | C-B-C | No | No | No |
| (01) | | | TACHYPLESIN- MAP30- | fragment | fragment | fragmen [symbol] |
| | | | ALLOFERON1 | | | |
| K5 | 36.55 kDa | 35 | C-B-D | No | No | No |
| | | | (GAEGURIN 5-MAP30-(KLAKLAK)2 | fragment | fragment | fragmen [symbol] |
| RetroMAD1 | 41.2 kDa | 1 | A-B-C | No | No | No |
| | | | (RETROCYCLIN 101- MAP30- | fragment | fragment | fragmen [symbol] |
| | | | DERMASEPTIN 1) | | | |
| RetroGAD1 | 35.29 kDa | 36 | A-B-C | No | No | No |
| | | | (RETROCYCLIN 101- GAP31- | fragment | fragment | fragmen [symbol] |
| | | | DERMASEPTIN 1) | | | |

[symbol]

indicates data missing or illegible when filed

TABLE 7A
Examples of fusion proteins according to the present invention

| SEQ | |
|---|---|
| ID | |
| NO: | SEQUENCE |
| 27 | [G-G-G-S]ₙ |
| 28 | SFGLCRLRRGFCAHGRCRFPSIPIGRCSRFVQCCRRVWVPGVGVPGVGGATGSDVNFDLSTATAKTYTK |
| | FIEDFRATLPFSHKVYDIPLLYSTISDSRRFILLNLTSYAYETISVAIDVTNVYVVAYRTRDVSYFFKE |
| | SPPEAYNILFKGTRKITLPYTGNYENLQTAAHKIRENIDLGLPALSSAITTLFYYNAQSAPSALLVLIQ |
| | TTAEAARFKYIERHVAKYVATNFKPNLAIISLENQWSALSKQIFLAQNQGGKFRNPVDLIKPTGERFQV |
| | TNVDSDVVKGNIKLLLNSRASTADENFITTMTLLGESVVNSCASRCKGHCRARRCGYYVSVLYRGRCYC |
| | KCLRCVPGVGVPGVG |
| 29 | LEKRKWKLFKKIEKVGQRVRDAVISAGPAVATVAQATALAKNVPGVGVPGVGGATGSDVSFRLSGATSK |
| | KKVYFISNLRKALPNEKKLYDIPLVRSSSGSKATAYTLNLANPSASQYSSFLDQIRNNVRDTSLIYGGT |
| | DVAVIGAPSTTDKFLRLNFQGPRGTVSLGLRRENLYVVAYLAMDNANVNRAYYFKNQITSAELTALFPE |
| | VVVANQKQLEYGEDYQAIEKNAKITTGDQSRKELGLGINLLITMIDGVNKKVRVVKDEARFLLIAIQMT |
| | AEAARFRYIQNLVTKNFPNKFDSENKVIQFQVSWSKISTAIFGDCKNGVFNKDYDFGFGKVRQAKDLQM |
| | GLLKYLGRPKSSSIEANSTDDTADVLVPGVGVPGVG KTCENLADTFRGPCFATSNC |
| 30 | MGRICRCICGRGICRCICGVPGVGVPGVGGSDVNFDLSTATAKTYTKFIEDFRATLPFSHKVYDIPLLY |
| | STISDSRRFILLDLTSYAYETISVAIDVTNVYVVAYRTRDVSYFFKESPPEAYNILFKGTRKITLPYTG |
| | NYENLQTAAHKIRENTDLGLPALSSAITTLFYYNAQSAPSALLVLIQTTAEAARFKYIERHVAKYVATN |
| | FKPNLAIISLENQWSALSKQIFLAQNQGGKFRNPVDLIKPTGERFQVTNVDSDVVKGNIKLLLNSRAST |
| | ADENFITTMTLLGESVVEFPW |
| 31 | MGSDVNFDLSTATAKTYTKFIEDFRATLPFSHKVYDIPLLYSTISDSRRFILLDLTSYAYETISVAIDV |
| | TNVYVVAYRTRDVSYFFKESPPEAYNILFKGTRKITLPYTGNYENLQTAAHKIRENTDLGLPALSSAIT |
| | TLFYYNAQSAPSALLVLIQTTAEAARFKYIERHVAKYVATNFKPNLAIISLENQWSALSKQIFLAQNQG |
| | GKFRNPVDLIKPTGERFQVTNVDSDVVKGNIKLLLNSRASTADENFTTTMTLLGESVVEFPWVPGVGVP |
| | GVGGRICRCICGRGICRCICG |
| 32 | MGSDVNFDLSTATAKTYTKFIEDFRATLPFSHKVYDIPLLYSTISDSRRFILLDLTSYAYETISVAIDV |
| | TNVYVVAYRTRDVSYFFKESPPEAYNILFKGTRKITLPYTGNYENLQTAAHKIRENTDLGLPALSSAIT |
| | TLFYYNAQSAPSALLVLIQTTAEAARFKYIERHVAKYVATNFKPNLAIISLENQWSALSKQIFLAQNQG |
| | GKFRNPVDLIKPTGERFQVTNVDSDVVKGNIKLLLNSRASTADENFITTMTLLGESVVEFPWVPGVGVP |
| | GVGALWKTMLKELGTMALHAGKAALGAAADTISQGTQ* |
| 33 | MALWKTMLKELGTMALHAGKAALGAAADTISQGTQVPGVGVPGVGGSDVNFDLSTATAKTYTKFIEDFR |
| | ATLPFSHKVYDIPLLYSTISDSRRFILLDLTSYAYETISVAIDVTNVYVVAYRTRDVSYFFKESPPEAY |
| | NILFKGTRKITLPYTGNYENLQTAAHKIRENTDLGLPALSSAITTLFYYNAQSAPSALLVLIQTTAEAA |
| | RFKYIERHVAKYVATNFKPNLAIISLENQWSALSKQIFLAQNQGGKFRNPVDLIKPTGERFQVTNVDSD |
| | VVKGNIKLLLNSRASTADENFITTMTLLGESVVEFPW* |
| 34 | VPGVGVPGVGKWCFRVCYRGICYRRCRVPGVGVPGVGGATGSDVNFDLSTATAKTYTKFIEDFRATLPF |
| | SHKVYDIPLLYSTISDSRRFILLNLTSYAYETISVAIDVTNVYVVAYRTRDVSYFFKESPPEAYNILFK |
| | GTRKITLPYTGNYENLQTAAHKIRENTDLGLPALSSAITTLFYYNAQSAPSALLVLIQTTAEAARFKYI |
| | ERHVAKYVATNFKPNLAIISLENQWSALSKQIFLAQNQGGKFRNPVDLIKPTGERFQVTNVDSDVVKGN |
| | IKLLLNSRASTADENFITTMTLLGESVVNVPGVGVPGVGHGVSGHGQHGVHG |
| 35 | VPGVGVPGVGFLPLLAGLAANFLPTIICFISYKCVPGVGVPGVGGATGSDVNFDLSTATAKTYTKFIED |
| | FRATLPFSHKVYDIPLLYSTISDSRRFILLNLTSYAYETISVAIDVTNVYVVAYRTRDVSYFFKESPPE |
| | AYNILFKGTRKITLPYTGNYENLQTAAHKIRENIDLGLPALSSAITTLFYYNAQSAPSALLVLIQTTAE |
| | AARFKYIERHVAKYVATNFKPNLAIISLENQWSALSKQIFLAQNQGGKFRNPVDLIKPTGERFQVTNVD |
| | SDVVKGNIKLLLNSRASTADENFITTMTLLGESVVNVPGVGVPGVGKLAKLAK KLAKLAK |

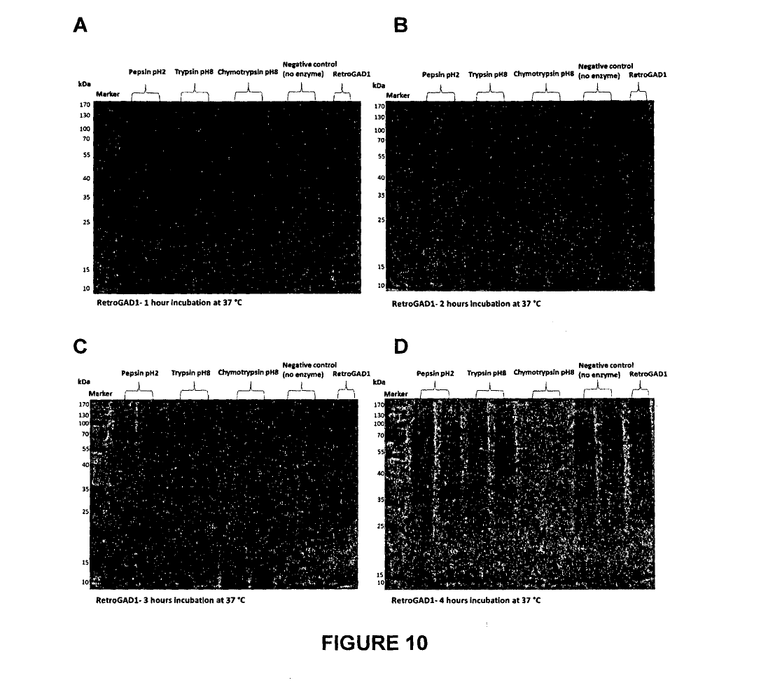

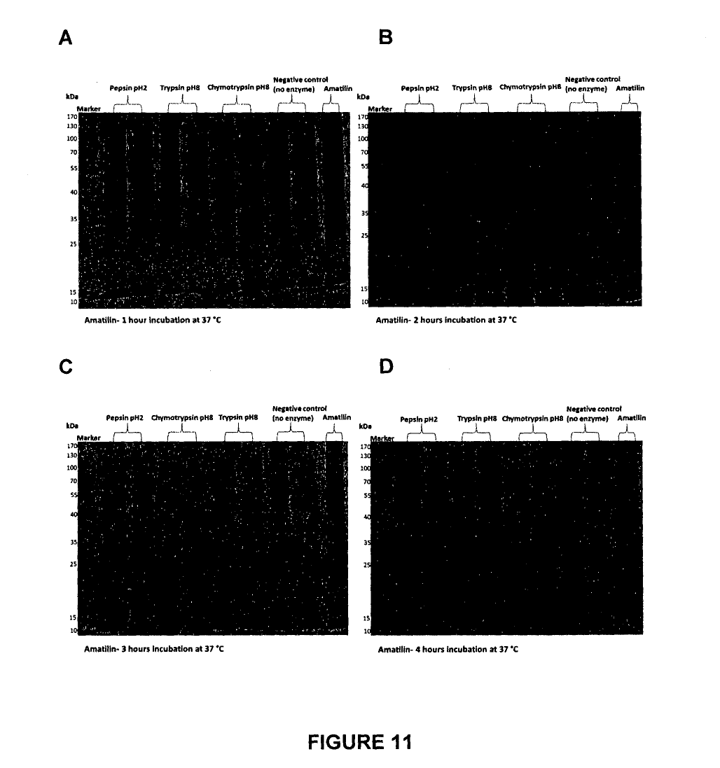

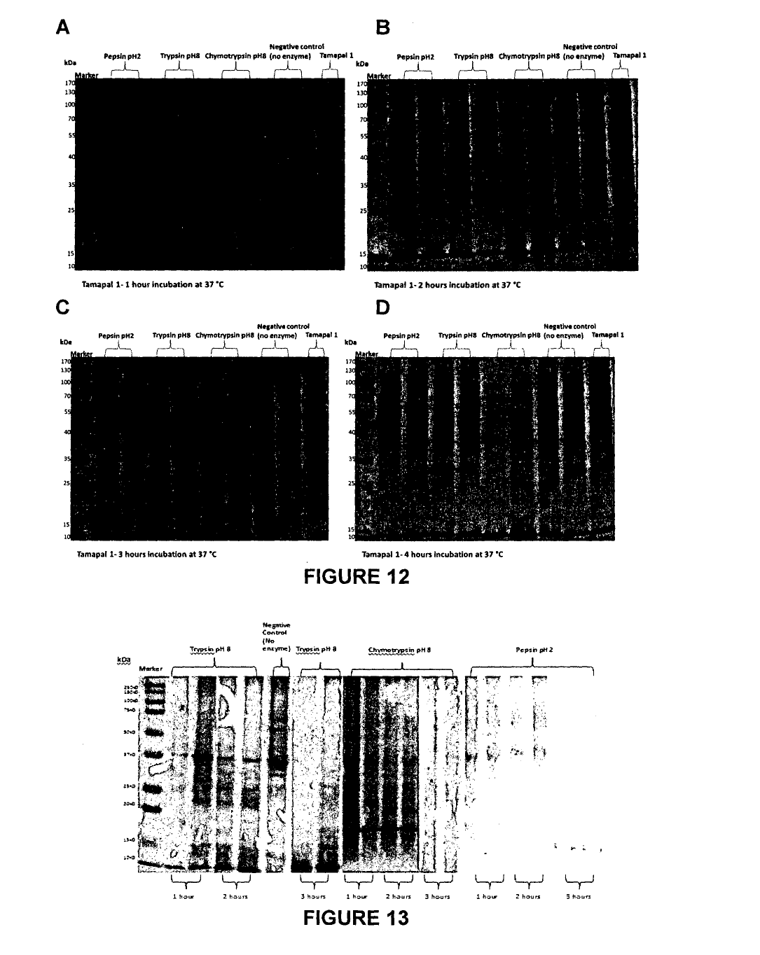

TABLE 7B
Summary of proteolytic digestion of RetroGAD1 (FIG. 10),
Amatilin (FIG. 11) and Tamapal1 (FIG. 12) for 1 hour, 2 hours,
3 hours and 4 hours at 37° C. And RetroMAD1 (FIG. 13)
for 1 hour, 2 hours and 3 hours at 37° C.

| | | Proteolytic enzyme | | |
|---|---|---|---|---|
| | | Pepsin | | Chymotrypsin |
| Drug | Time | (pH 2) | Trypsin (pH 8) | (pH 8) |
| RetroGAD1 | 1 hour | Not Digested | Not Digested | Digested |
| | 2 hours | Not Digested | Not Digested | Digested |
| | 3 hours | Not Digested | Not Digested | Digested |
| | 4 hours | Not Digested | Not Digested | Digested |
| Amatilin | 1 hour | Not Digested | Not Digested | Partially Digested |
| | 2 hours | Not Digested | Not Digested | Partially Digested |
| | 3 hours | Not Digested | Not Digested | Digested |
| | 4 hours | Not Digested | Not Digested | Digested |
| Tamapal1 | 1 hour | Not Digested | Not Digested | Partially Digested |
| | 2 hours | Not Digested | Not Digested | Partially Digested |
| | 3 hours | Not Digested | Not Digested | Partially Digested |
| | 4 hours | Not Digested | Not Digested | Digested |
| RetroMAD1 | 1 hour | Not Digested | Not Digested | Not Digested |
| | 2 hours | Not Digested | Not Digested | Not Digested |
| | 3 hours | Not Digested | Partially | Partially Digested |
| | | | Digested | |

The human G.I. is divided into the oral cavity, the stomach, the small intestines and the large intestines. Protease enzymes occur in the stomach, in the form of pepsin, and in the front part of the small intestines called the duodenum, in the form of trypsin and chymotrypsin. Pepsin is most active at pH 2 while trypsin and chymotrypsin are most active at pH 8. By running SDS-PAGE gels after incubation with the respective enzyme at its pH optima, single bands corresponding to the correct molecular size indicated that no enzymatic breakdown was observed for that period of incubation. Based on the results in the table 6 below, several compounds of this class demonstrated this attribute for a 2 hour incubation period with pepsin, trypsin and chymotrypsin individually because food does not normally retain in either the stomach or the duodenum for longer than 2 hours. This 2 hour incubation period for a drug to be orally administered before meals is far more than sufficient to prove stability within the G.I. with regard to enzymatic cleavage.

Conjugating these peptides with MAP30, surprisingly render the fusion protein stable for oral administration as shown in its ability to survive protease digestion.

RetroGAD1, Amatilin and Tamapal1 were not digested by pepsin (pH2) and trypsin (pH8) up to 4 hours post-digestion. Conversely, RetroGAD1, Amatilin and Tamapal1 were digested by chymotrypsin (pH8) at different points of time. RetroGAD1 was digested by chymotrypsin after 1 hour, Amatilin was only partially digested after 2 hours and digested after 3 hours, Tamapal1 was only partially digested after 3 hours and completely digested after 4 hours. For RetroMAD1, it was not digested by pepsin (pH2), chymotrypsin (pH8) and trypsin (pH8) up to 2 hours. This study indicates that RetroMAD1 and Tamapal1 are the most stable drugs, followed by Amatilin and RetroGAD1. Hence, the stability of three drugs under in vitro gastric conditions based on our study is RetroMAD1>Tamapal1>Amatilin>RetroGAD1. The significant outcome of this study is to develop an understanding on the stability of the drugs (RetroMAD1, RetroGAD1, Amatilin and Tamapal1) in human digestive system, thus allows oral drug delivery, to be recommended 1 hour before meals.

### Example 6

### Expression Profile of HSV-Infected Cells Treated with RetroMad1

4 Sets of Cells were Prepared

1. Vero Cells

2. Vero Cells+RetroMAD1

3. Vero Cells+Virus

4. Vero Cells+RetroMAD1+Virus

*Time point of the sample preparation is 72 hours

Vero cells (African Green monkey kidney cell line) were obtained from American Type Culture Collections, Rockville, Md. They were used as the host cells for HSV-2. The cells were cultured using Dulbeco's Modified Eagle Medium (DMEM), supplemented with 10% Foetal bovine serum (FBS).

Herpes simplex 2 (HSV-2) virus stocks were obtained by inoculating monolayer of Vero cells in a 75 cm² tissue culture flasks with virus in maintenance medium containing 2% FBS and the cells were allowed to continue propagating at 37° C. for 4 days until the cytopathic effect (CPE) are confirmed. The cells and supernatant were then harvested by gentle pipetting. The media was removed from the flasks. 4 mL of trypsin added to each flask and placed back in incubator for 5 minutes. The flasks were removed from incubator and 4 mL of media added to each flask to inactivate trypsin. Cells were collected into 15 mL tubes and spun at 3000 rpm for 5-10 minutes at room temperature. The supernatant was removed from 15 ml tubes and 5 mL of PBS added to each tube. The cells were resuspended in PBS to remove excess trypsin and media. The cells were spun at 3000 rpm for 5-10 minutes at room temperature. The supernatant was removed from tubes and 1 mL of fresh lysis buffer added to each tube. The cells were resuspended in fresh lysis buffer and place the tubes in at 4° C. for 2-4 hours. The cell lysates were transferred to 1.5 mL microcentrifuge tubes and spun at 40000 rpm for 1 hour at 4° C. The supernatant was finally removed and transferred to a clean microcentrifuge tube and the remaining lysate stored in −80° C. freezer. The protein concentration was determined according to the instructions of GE Healthcare 2D quant kit. A standard curve (0-50 μg) was prepared using 2 mg/ml BSA standard solution and the protein concentration determined using the standard curve. Drystrips were rehydrated according to a method known in the art and first dimension isoelectric focusing carried our using the IPGphor Regular Strip Holder. Equilibration was carried our and then second dimension gel electrophoresis carried out by preparing 12.5% stacking gel and placing the strips on top of the stacking gel. Filter paper was loaded with protein marker on the stacking gel by making a well and the gel run at 120V. Mass spectrometry analysis was then carried out by first staining the gels and then destaining them. The gels were analysed using PDQQuest Software. The gels obtained for the 4 sets of cells above were compared and the protein spots with at least 2 fold increase or decrease in intensity were picked. These protein spots were analysed using MALDI TOF-TOF and search against MASCOT database done to retrieve protein spot identity. MASCOT search results that gave protein scores greater than 51 were considered significant. UniProt was then used to identify the function of the protein.

The results, in particular, the ability of RetroMAD1 to up-regulate cellular pathways in normal and virally infected cells is shown in Table 7 below. Influence of gene expression at a cellular level is proof of RetroMAD1's ability to penetrate and be readily absorbed by cells.

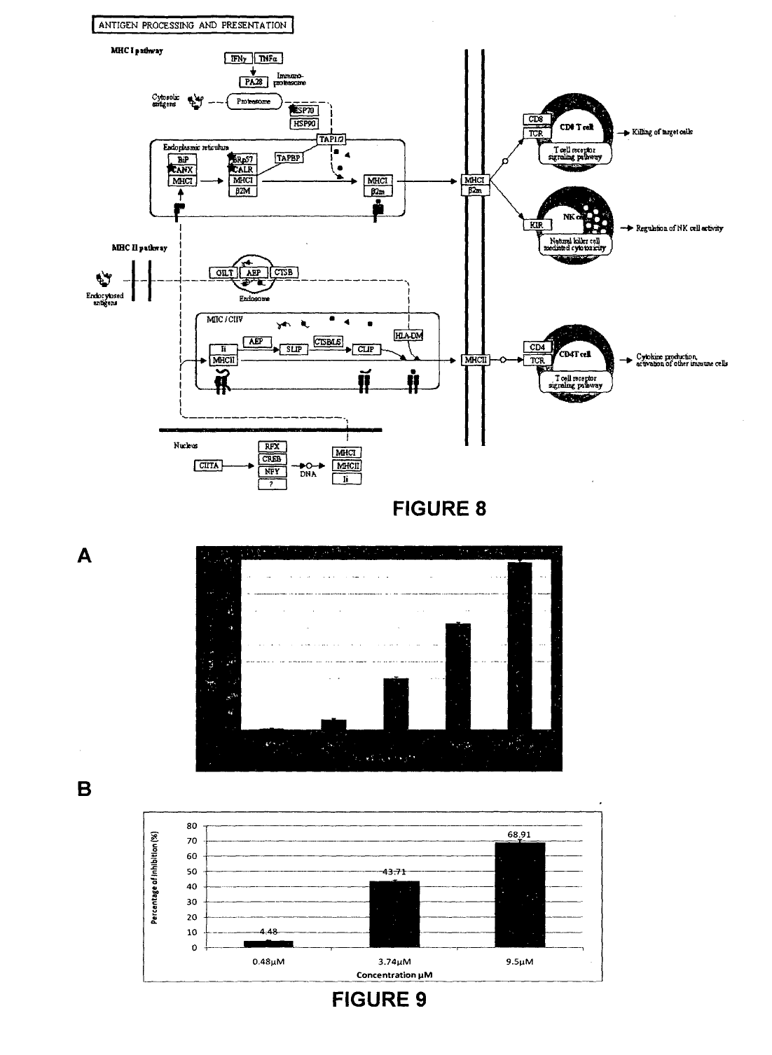

Viruses are known to hijack the cell's machinery to its advantages and major histocompatibility (MHC) class 1 antigen presentation molecules are usually targeted due to its important role in the immune system. From the Table 7it was evident that the virus had down-regulated the expression of proteins (sequestosome-1, calnexin, heat shock cognate, calreticulin, endoplasmin and protein disulfide-isomerase) involved in the MHC class I pathway. This was confirmed in FIG. 8 where the proteins were uploaded on david.abcc.ncifcrf.gov to produce the related pathways.

However, the expression of these proteins was augmented after the cells were treated with RetroMad1. Sequestosome-1, a protein responsible in the aggregation of a key initiator caspase, CASP8; was observed to be significantly up-regulated by as much as 11-fold. Alpha-enolase, a protein with glycolytic function as well as patholphysiological roles in many eukaryotes processes was also significantly suppressed by the virus. However, the expression of this protein was induced upon treatment with RetroMad1. In addition to alpha-enolase, annexin A1 was observed to be similarly repressed by the virus and its expression was restored upon treatment with the compound. Annexin A1 is a calcium-dependent phospholipid-binding protein which plays an important role in cellular processes such as proliferation and apoptosis as well as in preventing the fusion of raft-associated vesicles at selected membrane domains. Among the differentially expressed proteins, nucleoside diphosphate kinase with an ability in regulating cell cycle was also restored in treated cells and this is suggestive that RetroMad1 would be able to re-establish chromosomal stability in virally infected cells.

RetroMad1 is presumed to target the MHC class I pathway's proteins where it helps to re-establish the cell's ability in presenting viral peptides to the T-cells and ensure viral elimination in the immune system. Influence of gene expression at a cellular level is proof of RetroMAD1's ability to penetrate and be readily absorbed by cells.

TABLE 7
Expression profile of HSV-infected cells treated with RetroMad1

| Entrez | | | | RetroMad1 treated | Virally | RetroMad1 treated |
|---|---|---|---|---|---|---|
| ID | Protein Accession | Protein | Pathway involved | healthy cells | infected cells | virally infected cells |
| 8878 | SQSTM_PONAB | Sequestosome-1 | — | +1.01 | −8.47 | +11.06 |
| 821 | CALX_PONAB | Calnexin | Antigen processing and | −2.51 | −3.77 | +6.17 |
| | | | presentation, interaction in | | | |
| | | | MAPK3/ERK1 | | | |
| 811 | CALR_CHLAE | Calreticulin | Cell cycle | +2.56 | −1.07 | +3.65 |
| 3312 | HSP7C_SAGOE | Heat shock cognate | Antigen processing and | −1.80 | −9.07 | +2.00 |
| | | protein | presentation | | | |
| — | PDIA1_MACFU | Protein disulfide- | — | +1.87 | −5.03 | +2.03 |
| | | isomerase | | | | |
| — | ENPL_MACFA | Endoplasmin | IL6-mediated signaling | +2.02 | −3.64 | +4.34 |
| 2023 | ENOA_PONAB | Alpha-enolase | — | −1.56 | −6.32 | +1.30 |
| 301 | ANXA1_PANTR | Annexin A1 | — | −2.31 | −7.70 | +2.29 |
| — | NDKB_PONAB | Nucleoside diphosphate | — | +1.55 | −1.11 | +2.48 |
| | | kinase | | | | |
| 4691 | NUCL_PONAB | Nucleolin | — | −1.55 | −10.04 | +17.89 |

### Example 7

### Preliminary Screening Against Lung Cancer Cell Lines (A549) and Breast (MCF-7) Cancer Cell Lines

Normal and Cancer Cell Lines

Cell lines used in this study were established cell lines. The human breast carcinoma (MCF-7), human lung carcinoma (A549), human normal breast epithelium (184B5) and human normal bronchus epithelium (NL20) were purchased from the American Type Tissue Culture Collection, Manassas, USA. A549 and MCF-7 were grown in RPMI-1640 (Roswell Park Memorial Institute) and DMEM (Dulbecco's modified Eagles Medium), respectively while NL20 and 184B5 were grown in F-12K (ATCC, USA) and Mammary Epithelial Growth Medium (Lonza), respectively. Growth media was supplemented with 10% heat-inactivated foetal bovine serum (FBS, Gibco). Cells were maintained in humidified air with 5% CO₂ at 37° C. Cells undergoing exponential growth were used throughout the experiments.

Determination of Cell Viability, Growth Inhibition and Half-Maximal Inhibitory Concentration (IC₅₀)

The anti-proliferative activities of RetroMAD1 were measured using a colorimetric MTS assay which is composed of solutions of a novel tetrazolium compound 3-(4,5-dimethylthiazol-2-yl)-5-(3-carboxymethoxyphenyl)-2-(4-sulphonyl)-2H-tetrazolium, inner salt, MTS and an electron coupling reagent (phenazine methosulphate; PMS) (Promega, Madison, Wis.). This assay is based on the cleavage of the yellow dye MTS to purple formazan crystals by dehydrogenase activity in mitochondria, a conversion that occurs only in living cells. Prior to each experiment, cells from a number of flasks were washed thoroughly with phosphate buffered saline (PBS) (1×), harvested by treatment at 37° C. with a solution of Trypsin-EDTA (1×) and re-suspended in the culture medium. The cells were then counted and were seeded in each well of a 96-well flat-bottom plate at a concentration of 1×10⁴ cells/well for MCF-7, A549 and 184B5 cells and 2×10⁴ cells/well for NL20 cells. After 24 h of incubation at 37° C. with 5% CO₂, the cells were treated with various concentrations of RetroMAD1 for 24, 48 and 72 h. Control wells received culture medium without RetroMAD1 and blank wells contained culture medium with different concentrations of RetroMAD1 without cells. After 24, 48 and 72 h of incubation, cell proliferation was determined by the colorimetric MTS assay. Briefly, 20 μl per well of MTS reagent was added to the plates and incubated at 37° C. for 1 h in a humidified 5% CO₂ atmosphere. The intensity of formazan, reduced product of MTS after reaction with active mitochondria of live cells, was determined by measuring the absorbance at a wavelength of 490 nm using GloMax Multi Detection System (Promega, USA). Absorbance is directly proportional to the number of live cells in the culture. At least three replications for each sample were used to determine the anti-proliferative activity. Percentages of cell viability and growth inhibition were calculated using the following formulas:

$$Percentage of cell viability = \frac{[ \begin{matrix}Mean OD of the test group - \\ Mean OD of the blank group\end{matrix} ]}{[ \begin{matrix}Mean OD of the control group - \\ Mean OD of the blank group\end{matrix} ]} \times 100 \%$$

$$Percentage of growth inhibition = 100 \% - Percentage of cell viability$$

The IC₅₀ value (the concentration of drug that inhibits cell growth by 50% compared to untreated control) was determined from the dose response curve of the anti-proliferative activity with cell viability (Y-axis) against concentrations of RetroMAD1 (X-axis). Comparative study of the 24-hr IC50 values between a normal and a cancerous lung cell line gave an experimental Therapeutic Index of 2.94. The results are shown in Table 8 below.

TABLE 8
IC50 results of RetroMAD1 on breast can lung cancer cell lines.

| | IC₅₀ (μg/mL) of RetroMAD1 | | | |
|---|---|---|---|---|
| | Breast Cells | | Lung Cells | |
| | Cancer - | Normal - | Cancer - | Normal - |
| | Human breast | Human breast | Human lung | Human bronchus |
| | carcinoma | epithelium | carcinoma | epithelium |
| Time | (MCF-7) | (184B5) | (A549) | (NL20) |
| 24 h | 94.0 | ≦500.0 | 109.0 | 321.0 |
| 48 h | 78.5 | 180.0 | 80.0 | 254.0 |
| 72 h | 77.0 | 90.0 | 80.0 | 164.5 |

### Example 8

### Acute Toxicity Test in Imprinting Control Region (ICR) Mice

Adult male and female ICR mice (6-8 weeks old) were obtained from the Animal House, Faculty of Medicine, University of Malaya, Kuala Lumpur (Ethics No. PM 07/05/2008 MAA (a) (R). The mice weighed between 25-35 g. The animals were given standard rat pellets and tap water. The acute toxic study was used to determine a safe dose for RetroMAD1. Thirty six mice (18 males and 18 females) were separated into 3 groups and each group was fed orally once with (a) a vehicle only (normal saline, 5 ml/kg); (b) 0.105 mg/kg of RetroMAD1 prepared in normal saline and 1.05 mg/kg of RetroMAD1 of RetroMAD1 prepared in normal saline. The animals fasted overnight (food but not water) prior to dosing. Food was withheld for a further 3 to 4 hours after dosing. The animals were observed for 30 min and 2, 4, 8, 24 and 48 h after the administration for the onset of clinical or toxicological symptoms. Mortality, if any was observed over a period of 2 weeks. The animals were fasted on 14th day and sacrificed on the 15th day by an overdose of Ketamine anesthesia. Histological, hematological and serum biochemical parameters were determined following standard methods (Bergmeyer, 1980; Tietz et al., 1983). The results are shown in Table 9.

The study was approved by the ethics committee for animal experimentation, Faculty of Medicine, University of Malaya, Malaysia. All animals received human care according to the criteria outlined in the “Guide for the Care and Use of laboratory Animals” prepared by the National Academy of Sciences and published by the national Institute of health.

TABLE 9
Results of histological, hematological and serum biochemical
parameters obtained from carrying out test on the measured
from the control and tested ICR mice.

| Electrolytes/ | | | Low | Low | High | High |
|---|---|---|---|---|---|---|
| Renal | Control | Control | Dose | Dose | Dose | Dose |
| function tests | Male | Female | Male | Female | Male | Female |
| Sodium | 145.83 | 147.33 | 148.83 | 147.17 | 148 | 148.83 |
| Potassium | 7.57 | 5.67 | 7.18 | 5.18 | 6.93 | 5.77 |
| Chloride | 112.83 | 112 | 111 | 111 | 110.75 | 112.33 |
| Carbon | 16.08 | 16.07 | 18.5 | 18.02 | 18.05 | 15.05 |
| Dioxide | | | | | | |
| Anion Gap | 24.5 | 25 | 27.17 | 23.5 | 26.5 | 27.33 |
| Urea | 10.87 | 9.4 | 9.27 | 6.68 | 8.6 | 11.48 |
| Creatinine | 21.67 | 16 | 18.67 | 13 | 14 | 13 |
| Liver Function | | | | | | |
| Test | | | | | | |
| Total Protein | 51.33 | 53.67 | 50.5 | 55.17 | 51 | 53.17 |
| Albumin | 13.33 | 15.83 | 12.67 | 16.33 | 12 | 15.67 |
| Globulin | 38 | 37.83 | 37.83 | 38.83 | 39 | 37.5 |
| Total | 4.5 | 3.5 | 5 | 3.83 | 4.67 | 3.33 |
| Bilirubin | | | | | | |
| Conjugated | <1 | <1 | <1 | <1 | <1 | <1 |
| bilirubin | | | | | | |
| ALT | 28.83 | 29.17 | 36.67 | 30.17 | 32 | 32.5 |
| AST | 141.17 | 141.33 | 110 | 146.67 | 104.75 | 173.17 |
| ALP | 81.5 | 104.33 | 72.67 | 116.5 | 62.75 | 121.83 |
| G-Glutamyl | <3 | <3 | <3 | <3 | <3 | <3 |
| Transferase | | | | | | |
| Lipid Profile | | | | | | |
| Tests | | | | | | |
| Triglyceride | 0.91 | 1.2 | 1.47 | 1.4 | 1.62 | 1.46 |
| Total | 3.57 | 2.62 | 3.9 | 2.67 | 3.55 | 2.57 |
| Cholesterol | | | | | | |
| HDL | 3.26 | 2.55 | 3.44 | 2.6 | 3.1 | 2.46 |
| Cholesterol | | | | | | |
| LDL | −0.25 | −0.47 | −0.27 | 0.57 | 0.53 | −0.39 |
| Cholesterol | | | | | | |

The results of Table 9 show that there are no significant differences between male, female and between treated (low and high dose) and control. The results of the histopathology of liver and Kidney in ICR mice also showed no significant differences between male, female and between treated (low and high dose) and control. These results confirm that administration of RetroMAD1 may be considered non-toxic or at least minimally toxic on IRC mice to which the drug is administered at 50× and 500× the dose used to treat the cats and dogs, where significant symptomatic recovery was observed by 3 separate veterinary practitioners.

### Example 9

### Ability to Significantly Inhibit the Dengue Fever Virus NS2B-NS3 Polyprotein Protease

The reaction mixtures, with total volume of 200 μL, were prepared consisted of 100 μM fluorogenic peptide substrate (Boc-Gly-Arg-Arg-MCA), 2 μM DEN2 cNS2B-NS3pro complex, and with or without inhibitor (AVPs) of varying concentrations, buffered at pH 8.5 by 200 mM Tris-HCl. The RT-1 was initially prepared in Tris-HCl buffer and assayed at five different concentrations, i.e. 9.3-150 μM. The reaction mixtures without fluorogenic peptide substrate were firstly incubated at 280 C, 370 C and 400 C for 30 minutes each. Subsequently, the substrate was added and the mixture was further incubated at same temperatures for 30 minutes. Triplicates were performed for all measurements and the readings were taken using Tecan Infinite M200 Pro fluorescence spectrophotometer. Substrate cleavage was optimized at the emission at 440 nm upon excitation at 350 nm. The readings were then being used for calculating Km value of peptide substrate and IC50 values of peptide inhibitors using nonlinear regression model in GraphPad Prism 5.0 software. Ki values of competitive inhibitors were then being calculated as Ki=IC50/2 (Cheng and Prusoff, 1973).

The enzyme inhibition assay against the NS2B-NS3 shows that RetroMAD1 successfully caused a 98.64% inhibition at 15.1 μM with an IC50 value of 4.99 μM and a Ki value of 2.49 μM (FIG. 9A).

A second compound with a C-B-C configuration (SEQ ID NO:34) also gave promising results of 68.91% inhibition at 9.5 μM with an IC50 value of 4.74 μM and a Ki value of 2.37 μM (FIG. 9B).

### Example 10

### The Stability of RetroMAD1, RetroGAD1, Amatilin and Tamapal1

RetroGAD1, Amatilin and Tamapal1 were treated with different temperatures in the manner of fluctuations to examine the thermo-stability of the individual drugs. The drugs were incubated using a thermocycler (Labnet International, MultiGene Gradient) in high temperature for 15 mins then to a lower temperature for 45 minutes, as shown in the table below.

TABLE 10
Parameters of temperature fluctuations.

| Parameters | R1/T1 | | R2/T2 | | R3/T3 | | R4/T4 | |
|---|---|---|---|---|---|---|---|---|
| Temperature (° C.) | 60 | 50 | 55 | 45 | 50 | 40 | 70 | 55 |
| Time (mins) | 15 | 45 | 15 | 45 | 15 | 45 | 15 | 45 |

The fusion peptide solutions to be tested were loaded using a micropipette into 0.2 ml PCR tubes that were then placed into a thermocycler (Labnet International, MultiGene Gradient) which was then programmed to run at various temperature regimes as mentioned in Table 10. Each regime was made up of a short high temperature phase of 15 minutes followed by a longer medium temperature phase of 45 minutes. In these thermocycler trials, the harshest condition was a 15 minute 70° C. exposure followed by a 45 minute 55° C. exposure. Samples were then run on SDS-PAGE with the lanes as follows:—Lanes: M, marker; 1, negative control treated with 2×β-mercaptoethanol positive loading dye; 2, negative control treated with 2×β-mercaptoethanol negative loading dye; 3, Sample subjected to the temperature regime and treated with 2×β-mercaptoethanol positive loading dye; 4, sample subjected to the temperature regime and treated with 2×β-mercaptoethanol negative loading dye; 5, sample subjected to the temperature regime and treated with 2×β-mercaptoethanol positive loading dye; 6, Sample subjected to the temperature regime and treated with 2×β-mercaptoethanol negative loading dye. Comparison of the gel bands against the control gave a physical evidence as to whether the protein was damaged by the heat treatment or not.

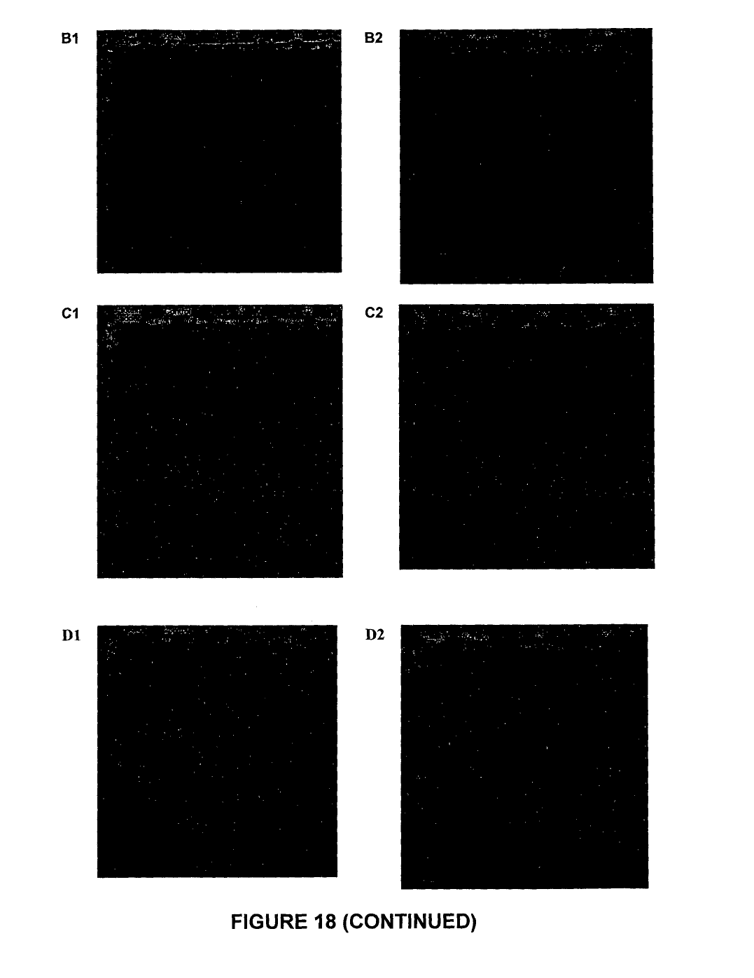

As can be seen in FIG. 18, all four drugs, RetroMAD1 (A1 and A2), RetroGAD1 (B1 and B2), Amatillin (C1 and C2) and Tamapal1 (D1 and D2) were intact under all treatments despite the presence of BME. This indicated that all four drugs were stable and were not affected by short-term temperature exposure of up to 70° C.

### Example 11

### Effects of Temperature on the Stability of Various Drugs Evaluated Via Antiviral Activity

Amatilin, RetroGAD1 and Tamapal1 were placed in −20, 4, 26 and 37° C. for up to 30 days. The peptides were also placed in 50° C. for up to 7 days. To further investigate the thermostability of the peptides, they were also exposed to four sets of various temperature fluctuations. Subsequently, the peptides were analyzed for their antiviral activity against HSV-2 via simultaneous treatment.

### Cytotoxicity of Tested Peptides on Vero Cells

The effect of Amatilin, RetroGAD1 and Tamapal1 on the growth of Vero cells was examined to rule out any direct cytotoxicity. Monolayer cultures of Vero cells were exposed to increasing concentrations of all the three peptides and after 24, 48 and 72 hours of incubation, cell viability was determined using MTS assay. Results are shown in Table 11 which indicate that the accepted maximal nontoxic concentrations (MNTD) of the three peptides on Vero cells were less than 20 μg/ml. At the chosen MNTD, the peptides did not impair the cell viability with respect to the untreated control group.

TABLE 11
Maximal non-toxic dose of the peptides on Vero cells

| | MNTD, ug/ml | | |
|---|---|---|---|
| Peptide | 24 h | 48 h | 72 h |
| Amatilin | 15 | 15 | 15 |
| RetroGAD1 | 10 | 10 | 10 |
| Tamapal1 | 15 | 15 | 15 |

### The Antiviral Activity of Peptides (Subjected to Various Temperatures) Against HSV-2

The antiviral activity of Amatilin, RetroGAD1 and Tamapal1 after incubation at different temperatures (−20, 4, 26, 37 and 50° C.) for 1, 7 and 30 days was evaluated by simultaneous treatment. For simultaneous treatment the mixture of the respective peptide and virus inoculated onto Vero cells and incubated for 24, 48 and 72 hours at 37° C. under 5% CO2 atmosphere. At the end of the time period the samples were harvested and viral DNA was extracted. The eluted DNA was then subjected to RT-PCR.

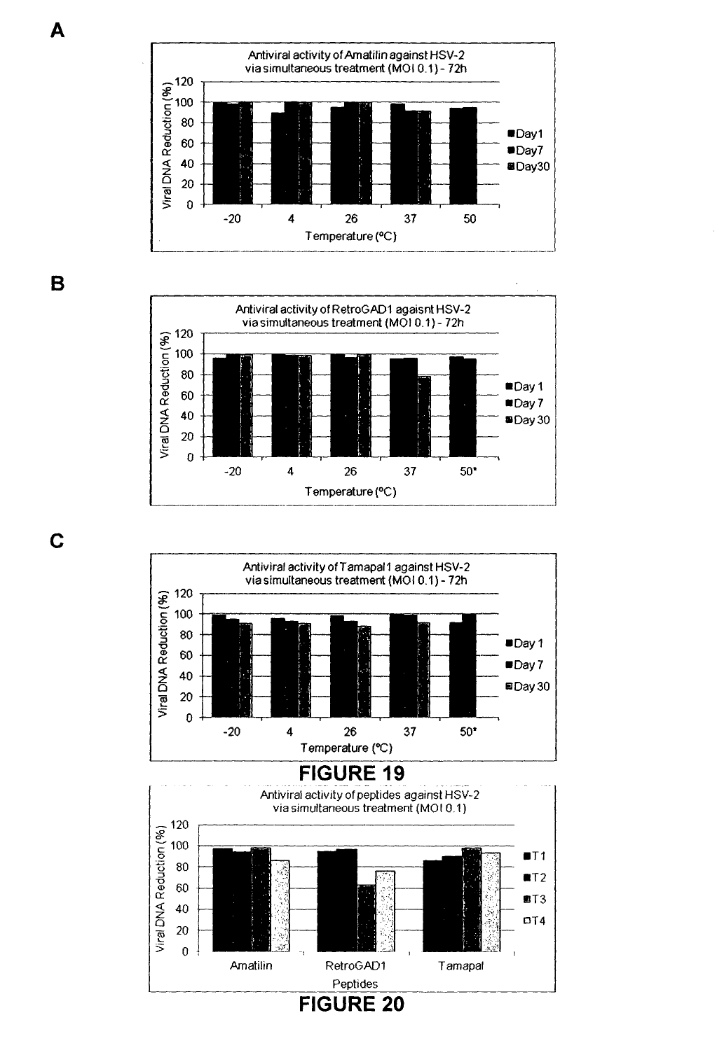

The results obtained suggested that all the three peptides were thermal stable. The peptides exposed to various temperatures for 1, 7 and 30 days showed strong inhibitory activity against HSV-2 via simultaneous treatment at the maximal non-toxic dose (MNTD). Amatilin was stable at high temperatures, 26 and 37° C. for up to 30 days giving 99.95 and 91.78% of inhibition, respectively. Amatilin was also stable at 50° C. for up to 7 days with 94.75% inhibitory activity (Table 12 and FIG. 19A). RetroGAD1 exhibited 99.01 and 78.52% inhibitory activity, respectively, after incubation at 26 and 37° C. for up to 30 days. The peptide showed 95.03% of viral reduction after incubation at 50° C. for up to 7 days (Table 12 and FIG. 19B). Tamapal1 was stable for up to 30 days at 26 and 37° C. giving 88.12 and 91.78% inhibitory activity, respectively. The peptide remained stable for 7 days at 50° C. with 99.42% of viral reduction (Table 12 and FIG. 19C).

TABLE 12
Percentage of viral reduction caused by Amatilin, RetroGAD1
and Tamapal1 incubated at different temperatures for 1, 7
and 30 days in simultaneous treatment determined by PCR.

| Tem- | Peptides | | | | | | | | |
|---|---|---|---|---|---|---|---|---|---|
| per- | Amatilin | | | RetroGAD1 | | | Tamapal1 | | |
| ature | Day | Day | Day | Day | Day | Day | Day | Day | Day |
| (° C.) | 1 | 7 | 30 | 1 | 7 | 30 | 1 | 7 | 30 |
| −20 | 99.84 | 98.00 | 99.98 | 95.93 | 98.94 | 98.96 | 98.73 | 94.77 | 91.01 |
| 4 | 89.35 | 99.98 | 99.92 | 99.66 | 98.92 | 98.30 | 95.84 | 92.92 | 90.87 |
| 26 | 94.53 | 99.75 | 99.95 | 99.77 | 96.24 | 99.01 | 98.49 | 92.92 | 88.12 |
| 37 | 98.55 | 91.45 | 91.78 | 95.54 | 95.61 | 78.52 | 99.45 | 99.16 | 91.78 |
| 50 | 94.31 | 94.95 | Not | 97.12 | 95.03 | Not | 91.36 | 99.42 | Not |
| | | | tested | | | tested | | | tested |

### The Antiviral Activity of Peptides (Subjected to Various Temperature Fluctuations Using Thermocycler) Against HSV-2

Amatilin, RetroGAD1 and Tamapal1 were exposed to four set of temperature fluctuations (T1, T2, T3 and T4) using thermocycler (Table 13). After exposure to various temperature fluctuations, the peptides were subjected to antiviral assay against HSV-2. The results obtained suggested that all the three peptides were thermally stable. Amatilin and Tamapal1 showed the strongest inhibitory activity against HSV-2 at all the four set of temperature fluctuations (Table 13 and FIG. 20).

TABLE 13
Percentage of viral reduction of HSV2 caused by Amatilin,
RetroGAD1 and Tamapal1 exposed to various temperature
fluctuations in simultaneous treatment determined by RT-PCR.

| | Peptides | | |
|---|---|---|---|
| Set of temperature fluctuations | Amatilin | RetroGAD1 | Tamapal1 |
| T1 | 97.28 | 94.88 | 86.21 |
| T2 | 94.05 | 96.95 | 90.36 |
| T3 | 97.85 | 63.04 | 97.64 |
| T4 | 86.00 | 75.91 | 93.65 |

### Example 12

NS2B and NS3 are two of seven non-structural proteins which may be translated from the single open reading frame (ORF) in a flavivirus RNA, and forms the serine protease complex NS2B-NS3. It is a crucial molecule in viral replication for processing non-structural regions and therefore is an attractive target for the development of antiviral drugs or compounds. An NS2B-NS3 protease assay using fluorogenic peptides was conducted to investigate the inhibitory characteristics of the drug against the protease at various concentrations and temperatures, using the method established by Rohana et. al. (2000).

Reaction mixtures were prepared with the following reagents: 2 μM isolated NS2B-NS3 protein complex from the DENV-2 viral genome, buffer at pH 8.5 (200 mM Tris-HCl) and different concentrations of the drugs respectively. After incubation at 37° C. for 30 minutes, 100 μM fluorogenic peptide substrate was added to the mixture, which was further incubated for another 30 minutes. Triplicates were performed for each concentration and readings were taken with a Tecan Infinite M200 Pro fluorescence spectrophotometer. Substrate cleavage was optimized at the emission of 440 nm upon excitation at 350 nm.

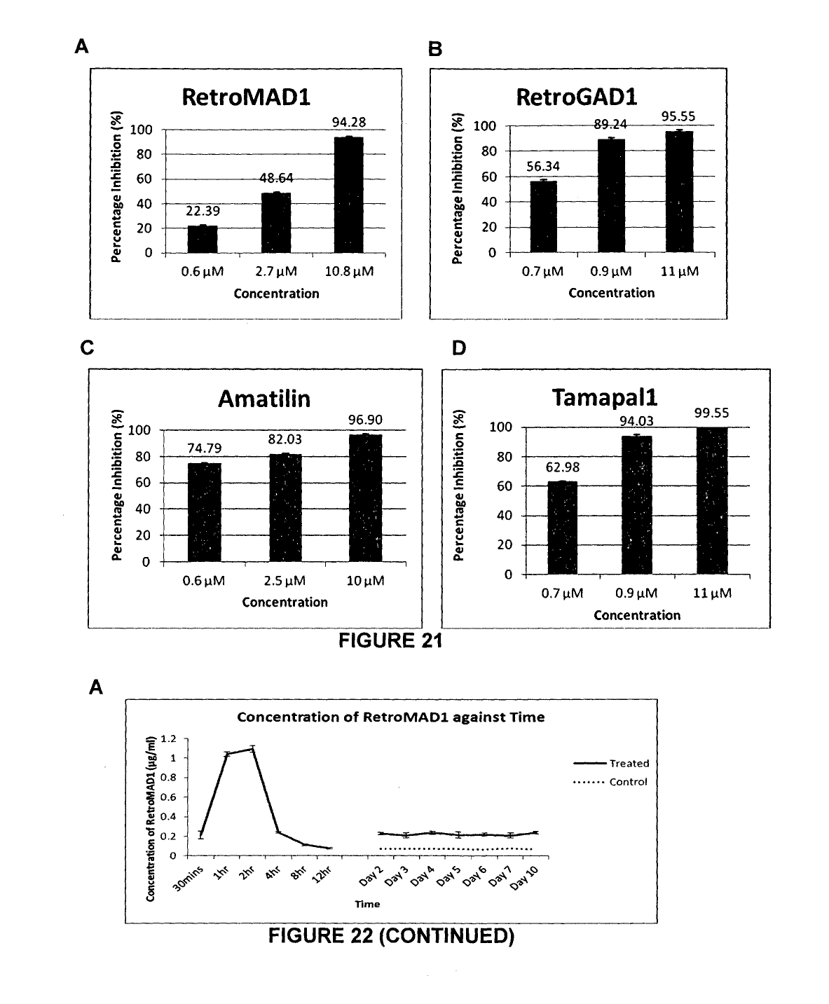

All of the drugs showed strong inhibition against this protease. Although RetroMAD1 has least inhibition activity against NS2B-NS3 compared to the other drugs, it managed to inhibit 94.28% of NS2B-NS3 at the concentration of 10.8 μM (FIG. 21A). RetroGAD1 inhibited 95.55% of NS2B-NS3 at 11 μM (FIG. 21B). Tamapal1 and Amatilin showed the strongest inhibition against NS2B-NS3 where more than 50% of NS2B-NS3 is inhibited by just using concentration of 0.7 μM and 0.6 μM respectively. At 10-11 μM of Tamapal1's and Amatilin's inhibition were nearly 100% of NS2B-NS3 (FIGS. 21C and D).

### Example 13

### Pharmacokinetic Study for Various Drugs of the Present Invention (RetroMAD1, RetroGAD1, Amatilin and Tamapal1)

Mice pK study is the study of the pharmacokinetics of the drug. pK includes study of the absorption, distribution, metabolism and excretion. Pharmacokinetics of RetroMAD1, RetroGAD1, Amatiin and Tamapal1 (as provided in Table 1c) was studied in ICR strain mice aged between 4-6 weeks.

The pharmacokinetic data of RetroMAD1, RetroGAD1, Amatiin and Tamapal1 was derived in 6-8 weeks female ICR mice. For each PK study for RetroMAD1, RetroGAD1, Amatiin and Tamapal1, 81 mice were administered with single dose of 70 ml per mouse which is a 50× dose of 0.2 mg/kg body weight for RetroMAD1, 0.7 ml per mouse for RetroGAD1, 0.6 ml per mouse for Amatiin, and 1 ml per mouse for Tamapal1. These drugs were given orally at time points, 0.5-, 1-, 2-, 4-, 8- and 12-hours on Day 1 and daily for Day 2, 3, 4, 5, 6, 7 and 10. Prior to administering the drug, the mice will be starved for 2 hours. At these time points, 0.5-, 1-, 2-, 4-, 8- and 12-hours on Day 1 and at Day 2, 3, 4, 5, 6, 7 and 10, 3 mice were fed orally with the drug (as treatment) and 3 mice were fed with water (as control). Before bleeding, each mouse was given 0.15 mL of anesthetic drug (Ketamine and Xylazine) via intraperitoneal injection. Each day blood samples were drawn from the heart of three treated mice and three controls at each time point. For the first day after the feed, the blood was collected after 30 min, 1 hour, 2 hours, 4 hours, 8 hours and 12 hours after oral administration and for the following days (up to day 10) the blood was collected just 30 min after administration. The blood samples were centrifuged and the serum was collected for ELISA. This was to determine the concentration of the drug in the blood system upon feeding (drug vs. water). Also, the organs including stomach, small and large intestine, liver and kidney were harvested. Harvested organs were homogenized in PBS and centrifuged to collect the supernatants. These supernatants were filtered and used for ELISA. Direct ELISA was used to determine concentration of RetroGAD1, Amatiin and Tamapal1 in the blood serum, stomach, liver, kidney and intestine, while a capture ELISA was used for RetroMAD1.

A direct ELISA was used for detecting RetroGAD1, Amatiin and Tamapal1 in mice Sera. In direct ELISA, a 96-well U-bottomed was coated with 5 μl of samples of mouse serum, supernatant of stomach, liver, kidney and intestine with 95 μl of coating buffer (0.2 M sodium carbonate-bicarbonate, pH 9.6). The sample coated plate was incubated at 4° C. overnight. Plates were washed six times with 0.05% Tween-20 in PBS 1×. 100 μl/well of rabbit anti-RetroGAD1/Amatiin/Tamapal1 antibody diluted 1:500 in 5% BSA in PBS and were added to the wells. After incubation at 37° C. for 1 hour, plates were washed similarly and 100 μl/well of anti-rabbit IgG diluted 1:10000 in 5% BSA in PBS was added. After incubation at 37° C. for 1 hour, plates were washed and 100 μl/well streptavidin—HRP diluted 1:10000 in 5% BSA in PBS was added. After incubation at 37° C. for 1 hour in the dark, plates were washed and 100 μl/well of OPD added to each well. Plates were incubated in the dark for 30 minutes at room temperature and reaction stopped with 50 μl/well of 4N H2SO4. Optical density (OD) for each sample was measured at 490 nm and 600 nm as background. A standard curve was then generated by doing the direct ELISA as described above with RetroGAD1, Amatiin and Tamapal1 of % dilution, the concentrations of RetroGAD1, Amatiin, and Tamapal1 at 100, 50, 25, 12.5, 6.25, 3.125, 1.6, 0.8, 0.4, 0.2 and 0.1 μg/ml. The equation of the standard curve was used to determine concentration of RetroGAD1, Amatiin and Tamapal1 in serum, stomach, liver, kidney and intestine.

ELISA for detecting RetroMAD1 in mice Sera is an in house Capture ELISA with anti-human-IgG-HRP. To prepare the capture antibody, a cat was fed daily with RetroMAD1 and after 6 months, blood was harvested and serum extracted. This serum was used as the capture antibody. 100 μl/well of this polyclonal cat anti-RetroMAD1 antibody diluted 1:80 in coating buffer (0.2 M sodium carbonate-bicarbonate, ph 9.6) was adsorbed onto 96-well polystyrene ELISA plates. The plates were incubated at 4° C. overnight. Plates were washed three times with 0.05% Tween-20 in PBS 1×. 100 μl/well of mice serum diluted 1:2 in 0.05% BSA in PBS and were added to the wells. After incubation at 37° C. for 1 hour, plates were washed similarly and 100 ul of anti RetroMAD1 positive human serum diluted 1:2000 in 0.05% BSA in PBS was added. After incubation at 37° C. for 1 hour, plates were washed and 100 μl/well Rabbit anti-human IgG HRP conjugate diluted 1:6000 in 0.05% BSA in PBS, was added. After incubation at 37° C. for 1 hour in the dark, plates were washed and 100 μl/well of OPD added to each well. Plates were incubated in the dark for 30 minutes at room temperature and reaction stopped with 50 μl/well of 4N H2SO4. Optical density (OD) for each sample was measured at 490 nm and 600 nm as background. All OD readings were then converted to Log values to obtain concentrations in μg/ml and the standard curves.

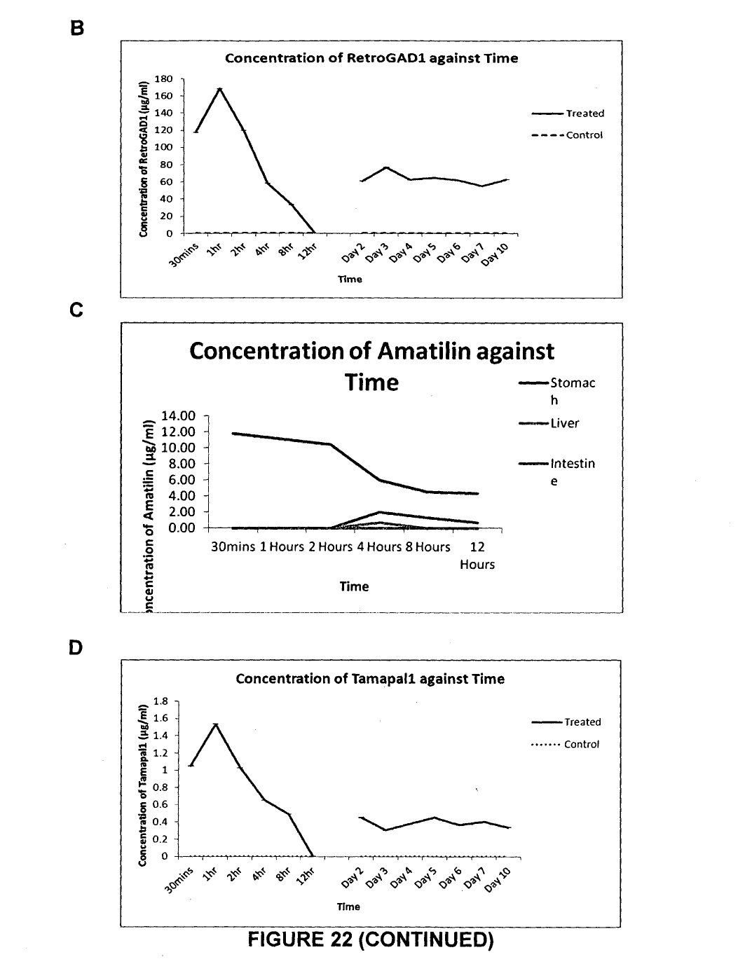

The mice pK results for RetroMAD1 are shown in FIG. 22A. The pK data showed that RetroMAD1 was detected in the serum as early as 30 minutes post feeding at about 0.2 μg/ml that reached a maximum at 1-2 hours at 1-1.1 μg/ml before dropping again to about 0.2 μg/ml at 4 hours. By 12 hours post feeding, levels were almost similar to the unfed controls indicating that the protein had been completely metabolized. Subsequent daily sampling at 30 minutes post feeding indicated levels around 0.2 μg/ml.

The mice pK data for RetroGAD1 are shown in FIG. 22B. The results showed that RetroGAD1 was detected in the serum as early as 30 minutes post feeding at about 118 μg/ml that reached a maximum at 1 hour at 169 μg/ml and 120 μg/ml before dropping again to 58.3 μg/ml at 4 hours and 33.7 μg/ml at 8 hours. By 12 hours post feeding, levels were similar to the unfed controls indicating that the drug had been completely eliminated from the blood. Subsequently daily sampling at 30 minutes post feeding indicated levels around 50 μg/ml.

The mice PK data for Amatilin as shown in FIG. 22C. The results showed that Amatilin was detected in the serum as early as 30 minutes post feeding at about 4.7 μg/ml that reached a maximum at 1 hour at 7.15 μg/ml and 7.52 μg/ml before decreasing again to 6.76 μg/ml at 4 hours and 3.84 μg/ml at 8 hours. By 12 hours post feeding, levels were similar to the unfed controls indicating that the drug had been completely eliminated from the blood. Subsequently daily sampling at 30 minutes post feeding indicated levels around 4 μg/ml.

The mice pK data for Tamapal1 are shown in FIG. 22D The results showed that Tamapal1 was detected in the serum as early as 30 minutes post feeding at about 1.05 μg/ml that reached a maximum at 1 hour at 1.54 μg/ml and 1.03 μg/ml before dropping again to 0.656 μg/ml at 4 hours and 0.493 μg/ml at 8 hours. By 12 hours post feeding, levels were similar to the unfed controls indicating that the drug had been completely eliminated from the blood. Subsequently daily sampling at 30 minutes post feeding indicated levels around 0.45 μg/ml.

Subsequent daily sampling 30 minutes post feeding levels around 0.2 μg/ml for RetroMAD1, 50 μg/ml for RetroMAD1, and 0.45 μg/ml Tamapal1, these data suggest acceptable bioavailability of the drugs.

### Example 14

### Organ Pharmacokinetics for RetroMAD1, RetroGAD1, Tamapal1 and Amatilin in Mice

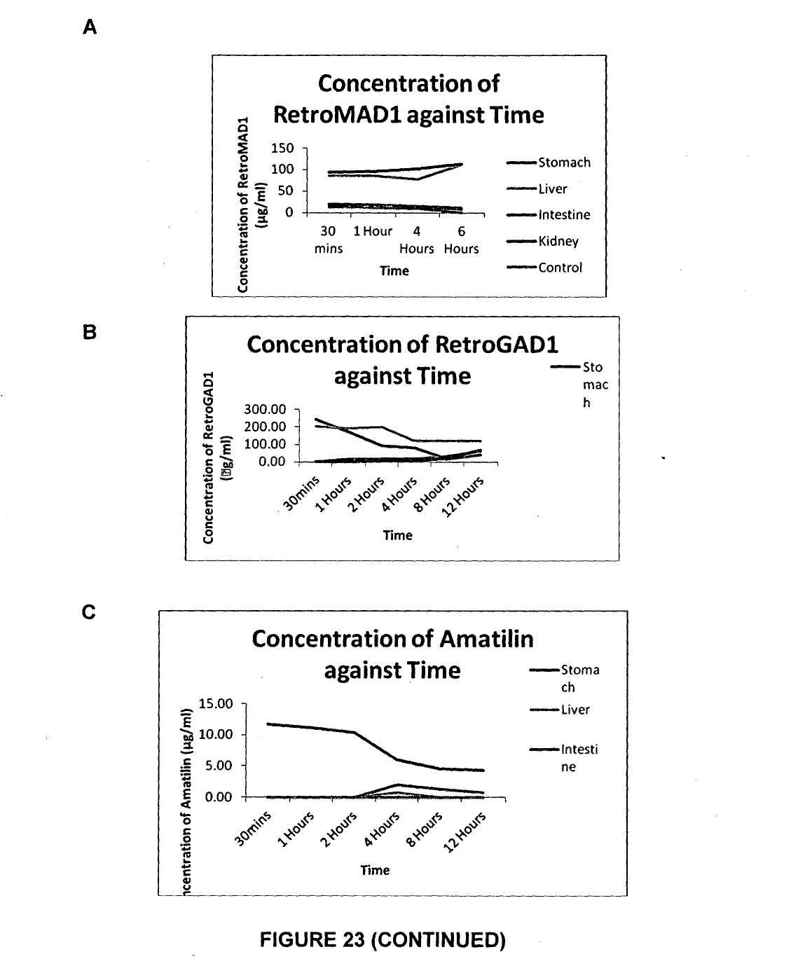

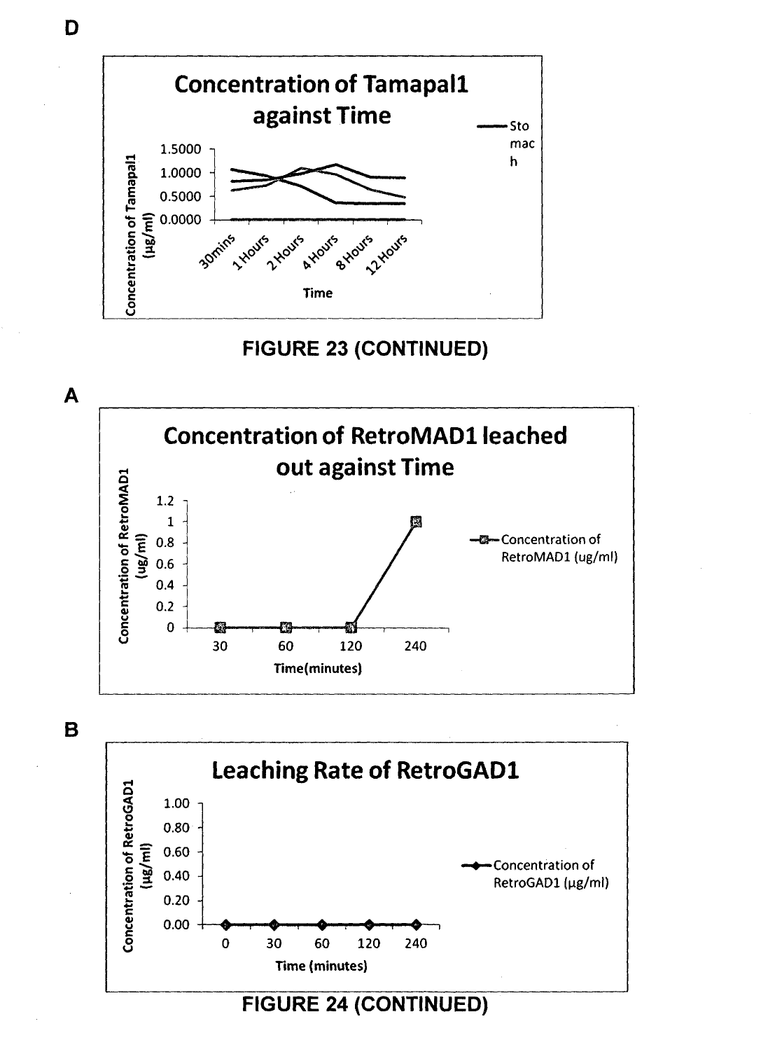

Mice Pk data of stomach, liver, kidney and intestine studies the pharmacokinetics of the drug. From the results as shown in FIGS. 23A-D after RetroGAD1, Amatilin and Tamapal1 are each orally given to mice and RetroMAD1 orally given to guinea pigs; these drugs were absorbed into the stomach and then distributed into the blood. Subsequently, metabolized and excreted in the kidney and intestine.

For RetroMAD1, pK study was carried out in guinea pigs. Data for guinea pigs small intestine supernatant is shown in Table 14 and FIG. 23A. Results showed that the highest concentration of RetroMAD1 was detected at 30 minutes at about 16 μg/ml. The concentration of RetroMAD1 then started to decrease to about 11 μg/ml at 1 hour, and to 9 μg/ml at 4 hours. The protein drug was then released from the small intestine at 6 hours where no RetroMAD1 was detected.

TABLE 14
Concentration of RetroMAD1 in guinea pig stomach, liver,
intestine and kidney after oral administration of RetroMAD1
at 30 mins, 1 hours, 4 hours and 6 hours

| | Concentration of RetroMAD1 (μg/ml) | | | | |
|---|---|---|---|---|---|
| Time | Stomach | Liver | Kidney | Small Intestine | Control |
| 30 mins | 20.33 | 86.28 | 94.59 | 15.91 | 13.58 |
| 1 Hour | 18.58 | 85.86 | 94.91 | 10.85 | 12.62 |
| 4 Hours | 14.86 | 78 | 102.68 | 9.98 | 14.07 |
| 6 Hours | 7.77 | 111.22 | 114.12 | 0 | 11.45 |

As for RetroGAD1, result showed (Table 15 and FIG. 23B) that the drug was absorbed into the stomach and blood system. The concentration of RetroGAD1 was detected at 30 minutes at 241.50 μg/ml in the stomach. Then the concentration in the stomach started to drop to about 170.47 μg/ml at 1 hour, and 92.62 μg/ml at 2 hours. RetroGAD1 was then released into the blood system at 1-2 hours and the concentration peaked at 1-2 hours at 169 μg/ml and 120 μg/ml. RetroGAD1 begun to increase in the liver from 2-4 hours and was detected to be 118.66 μg/ml. in the intestine, RetroGAD1 started to peak from 8 and 12 hours at 31.90 μg/ml and 60.15 μg/ml respectively. RetroGAD1 was also detected in the kidney at 22.02 μg/ml and 68.93 μg/ml at 8 hours and 12 hours respectively.

TABLE 15
Concentration of RetroGAD1 in stomach, liver and intestine at after oral
administration of RetroGAD1 at 0.5, 1, 2, 4, 8, 12 hours

| | Concentration of RetroGAD1 (μg/ml) | | | |
|---|---|---|---|---|
| Time | Stomach | Liver | Intestine | Control |
| 30 mins | 241.50 | 202.61 | 0.00 | 0.00 |
| 1 Hours | 170.47 | 192.71 | 16.63 | 0.00 |
| 2 Hours | 92.62 | 198.53 | 16.73 | 3.50 |
| 4 Hours | 80.10 | 118.66 | 18.60 | 3.50 |
| 8 Hours | 15.82 | 117.89 | 31.90 | 22.02 |
| 12 Hours | 41.13 | 117.89 | 60.15 | 68.93 |

TABLE 16
Concentration of Amatilin in stomach, liver, intestine and kidney at
after oral administration of Amatilin at 0.5, 1, 2, 4, 8, 12 hours

| | Concentration of Amatilin (μg/ml) | | | | |
|---|---|---|---|---|---|
| Time | Stomach | Liver | Intestine | Kidney | Control |
| 30 mins | 11.74 | 0.0000 | 0.0000 | 0.0000 | 0.0000 |
| 1 Hours | 11.11 | 0.0000 | 0.0000 | 0.0028 | 0.0020 |
| 2 Hours | 10.39 | 0.0000 | 0.0000 | 0.0035 | 0.0000 |
| 4 Hours | 5.95 | 0.7206 | 1.9679 | 0.0000 | 0.0000 |
| 8 Hours | 4.52 | 0.0000 | 1.3183 | 0.0056 | 0.0013 |
| 12 Hours | 4.32 | 0.0000 | 0.7139 | 0.0036 | 0.0007 |

TABLE 17
Concentration of Tamapal1 in stomach, liver, intestine and kidney at
after oral administration of Tamapal1 at 0.5, 1, 2, 4, 8, 12 hours

| | Concentration of Tamapal1 (μg/ml) | | | | |
|---|---|---|---|---|---|
| Time | Stomach | Liver | Intestine | Kidney | Control |
| 30 mins | 1.0666 | 0.6305 | 0.8114 | 0.0000 | 0.0000 |
| 1 Hours | 0.9357 | 0.7289 | 0.8514 | 0.0000 | 0.0000 |
| 2 Hours | 0.7156 | 1.0873 | 0.9822 | 0.0000 | 0.0000 |
| 4 Hours | 0.3635 | 0.9620 | 1.1676 | 0.0000 | 0.0001 |
| 8 Hours | 0.3487 | 0.6425 | 0.9097 | 0.0117 | 0.0000 |
| 12 Hours | 0.3480 | 0.4738 | 0.8927 | 0.0000 | 0.0001 |

As for Amatilin, results are shown (Table 16 and FIG. 23C) that it was absorbed into the stomach and blood system. The concentration of Amatilin was detected at 30 minutes at about 11.74 μg/ml in the stomach. The concentration of Amatilin in the stomach then starts to drop to about 11.11 μg/ml at 1 hour, and to 10.39 μg/ml at 2 hours. Amatilin is then release into the blood system at 1-2 hours, as Amatilin concentration peaks at 1-2 hours at 7.15 μg/ml and 7.52 μg/ml. Amatilin begins to increase in liver after 4 hours and was detected to be 0.7206 μg/ml. n the intestine, Amatilin started to increase from 4 to 8 hours at 1.97 μg/ml and 1.32 μg/ml. Amatilin was detected in the kidney at 0.0056 μg/ml and 0.0036 μg/ml at 8 hours and 12 hours respectively. As shown in the result, Amatilin is poorly detected in stomach, liver, intestine and kidney as direct ELISA for Amatilin is not completely optimized yet.

As for Tamapal1, result showed (Table 17 and FIG. 23D) that the drug was absorbed into the stomach and blood system. The concentration of Tamapal1 was detected at 30 minutes at about 0.716 μg/ml in the stomach. Then the concentration in the stomach was about 0.936 μg/ml at 1 hour, and 1.066 μg/ml at 2 hours. Tamapal1 was then released into the blood system at 1-2 hours, and the concentration peaked at 1-2 hours at 1.45 μg/ml and 1.03 μg/ml respectively. Tamapal1 begun to increase in the liver from 2 to 4 hours and was detected to be 1.087 μg/ml and 0.942 μg/ml. In the intestine, Tamapal1 started to peak from 8 and 12 hours at 0.982 μg/ml and 1.17 μg/ml respectively. Tamapal1 was also detected in the kidney at 0.0117 μg/ml at 8 hours and at 12 hours Tamapal1 was not detected.

### Example 15

### Leaching Rate of RetroMAD1, RetroGAD1, Amatilin and Tamapal1 from the Wafer Pellets Produced in a Pilot-Scale Manufacture

The leaching rate study for as the various fusion protein drugs as described in Example 7, was to study the time points when RetroMAD1, RetroGAD1, Amatilin and Tamapal1 were leached out from the wafers. Wafers containing the drugs were placed within in 30 ppt sea salt water in 1:100 weight to volume ratio. Shrimp wafer pellets were formed by extrusion using a Clextral BC45 twin-screw extruder that was sprayed post extrusion with the fusion protein drugs to be tested followed by a spray coating in a vacuum chamber with squid oil to serve as an outer hydrophobic layer to ‘lock-in’ the test drug as well as to serve an a feeding attractant for the shrimp. Addition of RetroMAD1 was added at the rate of 300 mg/kg of wafer pellets. At 0, 30, 60, 120 and 240 minutes, sea salt water was collected to determined the concentration of the fusion protein drugs that was leached out of the wafers into the sea salt water. Capture ELISA (Promega, Glomax Miltidetection System) was used to determine the concentration of RetroMAD1, while Direct ELISA was used for RetroGAD1, Amatilin and Tamapal1 Capture ELISA, a 96 U-bottom well plated was coated with 1:1000 of rabbit anti-RetroMAD1 antibody and was incubated at 4° C. overnight. The plate was then washed with PBS-Tween20 six times before adding the samples collected at time point 0, 30, 60, 120 and 240 minutes and incubated at 37° C. for an hour. Subsequently, 1:2500 human anti-RetroMAD1 antibodies were added to capture RetroMAD1 from the samples bound on the rabbit anti-RetroMAD1 antibody. While in direct ELISA, a 96 well U-bottomed plate was coated with the samples collected at time point 0, 30, 60, 120 and 240 minutes and incubated overnight at 4° C. The plate was then washed with PBS-Tween20 and added with 1:500 rabbit antibodies against RetroGAD1, Amatilin and Tamapal1 to capture the protein drug bound on the plate. Subsequently, 1:10000 anti-rabbit IgG were added to detect rabbit antibodies bind against the protein drugs. Absorbance was read at 490 nm and 600 nm. A standard curve of drug concentration against absorbance was plotted to determine the concentration of the drug in each sample.

Both RetroMAD1 and Tamapal1 began leaching out only after 120 minutes. Both Amatilin and RetroGAD1 did not show any signs of leaching even 240 minutes. This shows that since shrimp usually consume all their feed within 30-60 minutes, this method of oral administration of these fusion protein drugs is viable for the treatment of shrimp viruses. Furthermore, as shrimp digestion is trypsin rather than chymotrypsin dependent, it does not matter that the drug is presented along with the feed.

Antibodies toward RetroMAD1, RetroGAD1, Amatilin and Tamapal1 were raised in 4 rabbits respectively. In each immunization, rabbits were immunized intramuscularly with RetroMAD1, RetroGAD1, Amatilin and Tamapal1 in single dose of 0.6 ml per rabbit which is a dose of 0.2 mg/kg body weight for RetroMAD1, 0.9 ml per rabbit which is a dose of 0.25 mg/kg body weight for RetroGAD1, 0.8 ml per rabbit which is a dose of 0.25 mg/kg body weight for Amatilin and 1 ml per rabbit which is a dose of 0.25 mg/kg body weight for Tamapal1.

Prior to immunization, on Day 0, blood was drawn from the rabbits. Pre-bleed blood collected on Day 0 was used as the base line in determining the antibody titer in rabbit. After pre-bleeding the rabbit, first immunization was given according to the dosage per body weight as mentioned above. Rabbits were bled before giving another immunization on Day 7, Day 14, Day 28 and Day 35. Blood serum of rabbits collected on Day 7, Day 14, Day 28 and Day 35 was used to determine antibody titer against RetroMAD1, RetroGAD1, Amatilin and Tamapal1. On Day 38, antibody towards RetroMAD1, RetroGAD1, Amatilin and Tamapal1 raised in rabbits were harvested. In harvesting the rabbit antibody, before bleeding, each rabbit was given anesthesia (Ketamine and Xylazine) intravenously; the sedative dose was calculated using the following formula

Ketamine=(30×body weight of the rabbit)/(Concentration of Ketamine, 100 mg/ml)

Xylazine=(3×body weight of the rabbit)/(Concentration of Xylazine, 20 mg/ml)

50 ml of blood was collected from each rabbit. Blood was centrifuged at 4000 rpm for 15 minutes; blood serum containing antibody towards RetroMAD1, RetroGAD1, Amatilin and Tamapal1 was collected and kept in −20° C. for further use.

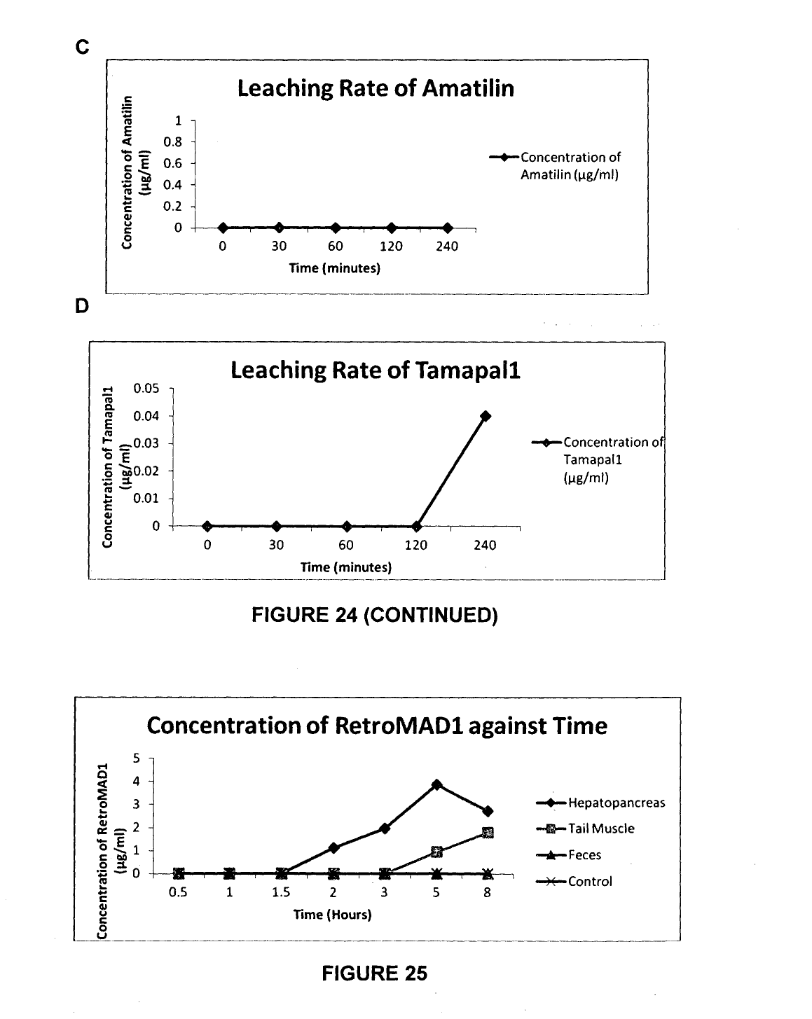

A direct ELISA was used to determine antibody titer in rabbit serum. A 96-well U-bottomed plate was coated with 1 μg/ml of RetroMAD1, RetroGAD1, Amatilin and Tamapal1 in coating buffer (0.2 M sodium carbonate-bicarbonate, ph 9.6). The sample coated plate was incubated at 4° C. overnight. Plates were washed six times with 0.05% Tween-20 in PBS 1×. 100 μl of 1/10 rabbit serum was added to the well, a 1/2 serial dilution of the rabbit serum was made. Rabbit serum was diluted in 1/10, 1/20, 1/40, 1/80, 1/160, 1/320, 1/640, 1/1280, 1/2560, 1/5120 and 1/10240 to determine the antibody titer. After incubation at 37° C. for 1 hour, plates were washed similarly and 100 μl/well of anti-rabbit IgG diluted 1:10000 in 5% BSA in PBS was added. After incubation at 37° C. for 1 hour, plates were washed and 100 μl/well streptavidin-HRP diluted 1:20000 in 5% BSA in PBS was added. After incubation at 37° C. for 1 hour in the dark, plates were washed and 100 μl/well of OPD added to each well. Plates were incubated in the dark for 30 min at room temperature and reaction stopped with 50 μl/well of 4N H₂SO₄. Optical densities (OD) were measured at 490 nm and 600 nm as background. The results are shown in FIGS. 24A-D and tables 18-21.

TABLE 18
Concentration of RetroMAD1 leached out against time

| Time (minutes) | Concentration of RetroMAD1 (μg/ml) |
|---|---|
| 30 | 0 |
| 60 | 0 |
| 120 | 0 |
| 240 | 1 |

TABLE 19
Concentration of RetroGAD1 leached out
at 0, 30, 60, 120 and 240 minutes

| Time (Minutes) | Concentration of RetroGAD1 (μg/ml) |
|---|---|
| 0 | 0.00000 |
| 30 | 0.00000 |
| 60 | 0.00000 |
| 120 | 0.00000 |
| 240 | 0.00000 |
| Control | 0.00000 |

TABLE 20
Concentration of Amatilin leached out
at 0, 30, 60, 120 and 240 minutes

| Time (Minutes) | Concentration of Amatilin (μg/ml) |
|---|---|
| 0 | 0.00000 |
| 30 | 0.00000 |
| 60 | 0.00000 |
| 120 | 0.00000 |
| 240 | 0.00000 |
| Control | 0.00000 |

TABLE 21
Concentration of Tamapal1 leached out
at 0, 30, 60, 120 and 240 minutes

| Time (Minutes) | Concentration of Tamapal1 (μg/ml) |
|---|---|
| 0 | 0.000000 |
| 30 | 0.000000 |
| 60 | 0.000000 |
| 120 | 0.000000 |
| 240 | 0.040167 |
| Control | 0.000000 |

### Example 16

### Short-Term Pharmacokinetics of RetroMAD1 in Shrimp Using Capture ELISA

In the short-term feeding study, shrimps were fed with 0.06 g of shrimp wafer pellets containing RetroMAD1 at an inclusion of 300 mg/kg. This study is to determine the short term kinetics of RetroMAD1 in terms of absorption, retention and excretion. Shrimp wafer pellets were formed by extrusion using a Clextral BC45 twin-screw extruder that was sprayed post extrusion with the fusion protein drugs to be tested followed by a spray coating in a vacuum chamber with squid oil to serve as an outer hydrophobic layer to ‘lock-in’ the test drug as well as to serve an a feeding attractant for the shrimp. Addition of RetroMAD1 was added at the rate of 300 mg/kg of wafer pellets.

Healthy specimens of the commonly cultured Pacific white shrimp Penaeus vannamei were selected from a shrimp farm in Tawau, Sabah, Malaysia and a single specimen ranging from 2.4-5.8 g was placed in each transparent plastic aquarium tank of 10 litres total capacity containing 5 litres of seawater at 32 parts per thousand salinity. Specimens were acclimated for a week prior to the experiment and 50% water was changed daily by siphoning. A single airstone was provided such that aeration was sufficiently provided such that the animal did not display any signs of being stressed. A plastic netting was provided on top to prevent the specimens from jumping out. For each sampling time point, tanks were present in triplicate as in Group 1,2 and 3. As there were 8 sampling time points, 24 tanks were prepared as shown in the Table 22.

TABLE 22
Experiment design for measuring short-term
pharmacokinetics of RetroMAD1 in shrimp

| Sampling Points | Number of Shrimp per tank | | |
|---|---|---|---|
| Time (Hours) | Group 1 | Group 2 | Group 3 |
| Control | 1 | 1 | 1 |
| 0.5 | 1 | 1 | 1 |
| 1 | 1 | 1 | 1 |
| 1.5 | 1 | 1 | 1 |
| 2 | 1 | 1 | 1 |
| 3 | 1 | 1 | 1 |
| 5 | 1 | 1 | 1 |
| 8 | 1 | 1 | 1 |

At each sampling time point, the feces were collected by siphoning, the shrimp dissected removing the hepatopancreas well as the muscle of the last abdominal segment of the tail which was stored in PBS buffer and stored at −40° C. Note that the Control were fed normal shrimp pellets without RetroMAD1. The shrimp were unfed for the duration of the experiment after completely ingesting the test and control feeds. The weights of the feces, hepatopancreas and tail muscle (only the last abdominal segment) collected are presented in the table below.

TABLE 23
Weight of each shrimp, hepatopancreas, tail
muscle (last segment only) and feces

| | | Weight (grams) | | | |
|---|---|---|---|---|---|
| Time | | Whole | | Tail | |
| (Hour) | Tank | Shrimp | Hepatopancreas | Muscle | Feces |
| 0 (Control) | 1 | 3.238 | 0.120 | 0.108 | 0.000 |
| | 2 | 3.227 | 0.160 | 0.127 | 0.038 |
| | 3 | 3.554 | 0.190 | 0.231 | 0.043 |
| 0.5 | 1 | 3.600 | 0.250 | 1.960 | 0.045 |
| | 2 | 2.440 | 0.170 | 0.142 | 0.040 |
| | 3 | 3.720 | 0.187 | 0.136 | 0.020 |
| 1 | 1 | 2.785 | 0.152 | 0.100 | 0.065 |
| | 2 | 3.213 | 0.141 | 0.133 | 0.030 |
| | 3 | 4.236 | 0.211 | 0.208 | 0.015 |
| 1.5 | 1 | 2.130 | 0.126 | 0.095 | 0.070 |
| | 2 | 4.117 | 0.175 | 0.206 | 0.053 |
| | 3 | 1.612 | 0.100 | 0.083 | 0.086 |
| 2 | 1 | 5.500 | 0.236 | 0.222 | 0.041 |
| | 2 | 2.784 | 0.155 | 0.116 | 0.052 |
| | 3 | 2.993 | 0.182 | 0.160 | 0.056 |
| 3 | 1 | 3.538 | 0.190 | 0.142 | 0.024 |
| | 2 | 3.719 | 0.175 | 0.154 | 0.083 |
| | 3 | 3.995 | 0.199 | 0.147 | 0.054 |
| 5 | 1 | 3.508 | 0.154 | 0.188 | 0.060 |
| | 2 | 3.962 | 0.194 | 0.200 | 0.016 |
| | 3 | 2.443 | 0.155 | 0.104 | 0.050 |
| 8 | 1 | 4.995 | 0.245 | 0.245 | 0.035 |
| | 2 | 5.840 | 0.182 | 0.292 | 0.034 |
| | 3 | 3.460 | 0.148 | 0.146 | 0.038 |

Captured ELISA (Promega, Glomax Multidetection System) was used to determine concentration of RetroMAD1 in the samples. The tail muscle sampled was in the last abdominal segment after the anus to ensure any result did not come from the GI tract.

In captured ELISA, a 96 U-bottom well plated was coated with 1:1000 of rabbit anti-RetroMAD1 antibody and was incubated at 4° C. overnight. Plate was then washed with PBS-Tween20 six times before adding the samples of hepatopancreas, tail muscle and feces and incubated at 37° C. for an hour. Subsequently, 1:2500 human anti-RetroMAD1 antibody was added to capture RetroMAD1 from the samples bound on the rabbit anti-RetroMAD1 antibody. Absorbance was read at 490 nm and 600 nm. A standard curve of concentration of RetroMAD1 (μg/ml) against absorbance as shown in Table 23 was plotted to determine the concentration of RetroMAD1 in each sample.

Table 24 and FIG. 25 shows that hepatopancreal absorption of RetroMAD1 was detectable at 1.5 hours post-feeding and peaked at 5 hours post-feeding while RetroMAD1 was detectable in the tail muscle as early as 3 hours post-feeding.

TABLE 24
Concentration of RetroMAD1 against time

| | Treated with RetroMAD1 | | | |
|---|---|---|---|---|
| Time (hours) | Hepatopancreas | Tail muscle | Feces | Control |
| 0.5 | 0 | 0 | 0 | 0 |
| 1 | 0 | 0 | 0 | 0 |
| 1.5 | 0 | 0 | 0 | 0 |
| 2 | 1.133 | 0 | 0 | 0 |
| 3 | 1.983 | 0 | 0 | 0 |
| 5 | 3.867 | 0.9333 | 0 | 0 |
| 8 | 2.717 | 1.783 | 0 | 0 |

### Example 17

### Long-Term Pharmacokinetics of RetroMAD1 in Shrimp Using Capture ELISA

In the long-term feeding study, shrimps were fed with 0.2 g of shrimp pellets containing RetroMAD1 at 300 mg/kg inclusion rate. This study is to further determine the pharmacokinetics of RetroMAD1 in terms of absorption, retention and excretion over 7 days.

In this study, Healthy specimens of the commonly cultured Pacific white shrimp Penaeus vannamei were selected from a shrimp farm in Tawau, Sabah, Malaysia and 8 pcs of 10.0 g+/−0.5 g specimen were placed in a single transparent plastic aquarium tank of 50 litres total capacity containing 40 litres of seawater at 32 parts per thousand salinity. Specimens were acclimated for a week prior to the experiment and 50% water was changed every alternate day by siphoning. A single airstone was provided such that aeration was sufficiently provided such that the animal did not display any signs of being stressed. A plastic netting was provided on top to prevent the specimens from jumping out. Feces were collected daily by siphoning and placed into a 1.5 ml plastic tube that was capped and stored at −40° C. until required.

For each sampling time point, tanks were present in triplicate as in Group 1, 2 and 3. As there were 7 sampling time points as well as one control, 24 tanks were prepared each with 8 specimens as shown in Table 25

TABLE 25
Experiment design for measuring long-term
pharmacokinetics of RetroMAD1 in shrimp

| Sampling Points | Number of Shrimps per tank | | |
|---|---|---|---|
| Time (Days) | Group 1 | Group 2 | Group 3 |
| Control | Control 1 | Control 2 | Control 3 |
| 1 | 8 | 8 | 8 |
| 2 | 8 | 8 | 8 |
| 3 | 8 | 8 | 8 |
| 4 | 8 | 8 | 8 |
| 5 | 8 | 8 | 8 |
| 6 | 8 | 8 | 8 |
| 7 | 8 | 8 | 8 |

Within the first day, there were 6 sampling points at 2, 4, 6, 8, 12 and 16 hours. As there were 8 animals, one was removed at each sampling point and the feces and dissected hepatopancreas as well as the tail muscle from the last abdominal segment pooled and kept in PBS at −40° C. till required. The sampling point at 24 hours was taken at the start of Day 2 while the 36 hours sampling point taken mid-way through Day 2. Thereafter, there were sampling points daily. Captured ELISA (Promega, Glomex Multidetection System) was used to determine concentration of RetroMAD1 in hepatopancreas, tail muscle and faeces, in the method previously described earlier.

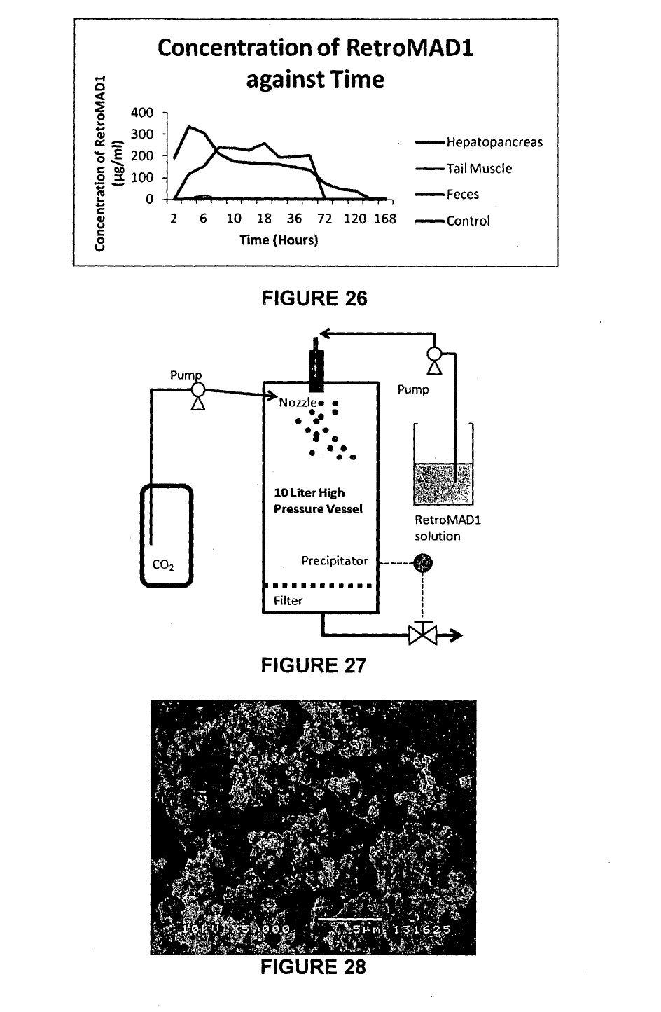

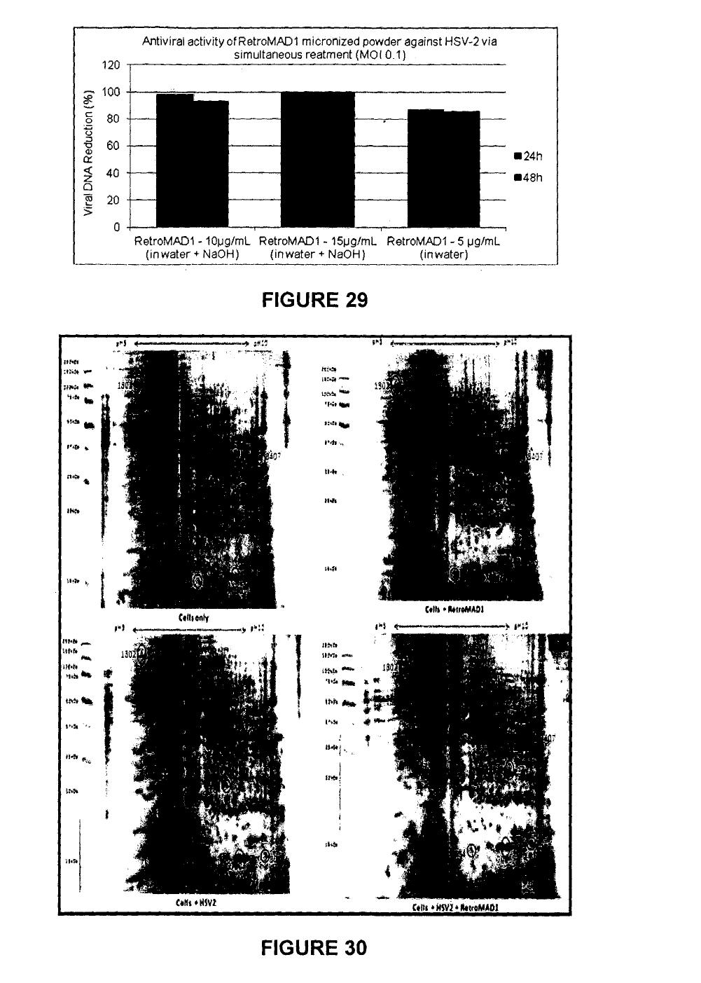

Table 26 and FIG. 26 showed that hepatopancreal RetroMAD1 peaked after 5 hours in agreement with the previous experiment (Example 15). Residual RetroMAD1 could be detected up to 144 hours (day 6) even after faecal RetroMAD1 was no long detectable after 72 hours (day 3). Detectable RetroMAD1 in the tail muscle remained only for 8 hours showing that the drug residue is quickly broken down by active swimming shrimp.

This indicated a safe ‘withdrawal period’ of 14 days for head-on shrimp product (sold with the head containing the hepatopancreas) and 7 days for a headless shrimp product (where the head containing the hepatopancreas is removed at the processing factory prior to freezing.

TABLE 26
Concentration of RetroMAD1 in hepatopancreas,
tail muscle and feces at 2, 4, 6, 8, 10, 12, 18,
24, 36, 48, 72, 96, 120, 144 and 168 hours.

| | Concentration of RetroMAD1 (μg/ml) | | | |
|---|---|---|---|---|
| Time (Hours) | Hepatopancreas | Tail Muscle | Feces | Control |
| 2 | 189.62 | 0 | 0 | 0 |
| 4 | 335.085 | 5.272 | 115.751 | 0 |
| 6 | 304.758 | 16.015 | 151.794 | 0 |
| 8 | 209.824 | 0 | 238.178 | 0 |
| 10 | 174.754 | 0 | 234.733 | 0 |
| 12 | 168.082 | 0 | 224.924 | 0 |
| 18 | 165.378 | 0 | 256.926 | 0 |
| 24 | 160.538 | 0 | 194.13 | 0 |
| 36 | 147.025 | 0 | 195.96 | 0 |
| 48 | 136.772 | 0 | 201.383 | 0 |
| 72 | 75.827 | 0 | 0 | 0 |
| 96 | 49.567 | 0 | 0 | 0 |
| 120 | 40.343 | 0 | 0 | 0 |
| 144 | 3.275 | 0 | 0 | 0 |
| 168 | 3.426 | 0 | 0 | 0 |

### Example 18

### Field Trial Against CPV2 in Puppies in Manila as an Orally Administered Drug

32 confirmed CPV2 client-puppies were recruited by Registered Vets in Manila, using CPV Ag Test Kits and were divided into two groups: group A (11 patients) treated with standard of care (supportive and symptomatic treatments); group B (21 patients) treated with RetroMAD1 and standard of care. This was considered to be a “Death-model” experimental trial as up to 80-90% of untreated puppies would have died of this virus within a week (Table 27).

TABLE 27
54.55% of patients recovered from CPV2 in Group A.
In Group B, 80.95% of patients recovered from CPV.

| | Group A | Group B |
|---|---|---|
| Total number of patients | 11 | 21 |
| Number of patients recovered | 6 | 17 |
| Percentage recovery | 54.55% | 80.95% |

Supportive and symptomatic treatment included the use of crystalloid intravenous fluid for total parenteral nutrition and to address dehydration. The use of anti-emetics such as metoclopramide and ranitidine was allowed. Antibiotics such as amoxicillin, enrofloxacin, and marbofloxacin were used to address secondary bacterial infection. The use of anti-diarrheal drugs was not allowed because it may worsen the effects of disease. The use of other antiviral drugs such as oseltamivir, recombinant human granulocyte stimulating factor (G-CSF), NSAID's such as flunixin meglumine, and anti-TNF was discouraged, however, if the veterinarian believes there was a necessity to do so, it was allowed and was noted.

RetroMAD1 showed promising results in treating CPV2. The percentage of recovery increased from 54.55% to 80.95% when treated with RetroMAD1 in addition to the standard of care compared to the patients treated with standard of care only (Table 27). This showed that oral delivery is proven possible.

### Companion Animal Multicenter Trials

Multicenter trials were carried out by Veterinary Surgeons in Malaysia, Singapore and Philippines namely, Dr. Tan Thiam Khoon (Malaysia), Dr. B. P. M. Mohanakrishnan (Malaysia), Dr. V. C. Vasavan (Malaysia), Dr. Kiew C. X (Malaysia), Dr. Chan Kah Yein (Malaysia), Dr. Frederic Chua (Singapore), Dr. Carlo Peralta (Philippines), and two animal rescuers from Malaysia.

Diagnosis was made by the veterinarian to confirm the pathology of the disease of each highly symptomatic animal before and after the animal was treated with RetroMAD1. A report was done for each animal and signed off by the respective vet. The end point outcome was that the veterinarians acknowledged clearly evident prolonged symptomatic relief in a significant majority of cases which is unusual for these diseases.

This arrangement was a “Data for Drug” scheme where RetroMAD1 was given to the veterinarian in exchange for results obtained from client—animals with owner's consent.

The dosage given to the animal was the efficacy of 0.2 mg/kg of body weight for 1-3 weeks depending on disease severity. From the results obtained, supportive evidence of oral delivery of RetroMAD1 is further provided with a high percentage of symptomatic recoveries except for FIV-FeLV co-infection and FIPV in cats (Table 28).

TABLE 28
The compiled results from all the multicenter trials.

| | Disease | Cases | % Recovery |
|---|---|---|---|
| Cats | Feline Immunodeficiency Virus (FIV) | 20 | 75 |
| | Feline Leukemia Virus (FeLV) | 27 | 74 |
| | FIV FeLV co-infection | 8 | 37.5 |
| | Feline Panleukopenia Virus (FPV) | 10 | 90 |
| | Feline infectious peritonitis virus (FIPV) | 20 | 10 |
| | Feline Viral Rhinotracheitis (FVR) | 6 | 83.3 |
| | Feline Calicivirus (FCV) | 8 | 75 |
| Dogs | Canine Parvovirus type 2 (CPV2) | 140 | 80 |

### Example 19

### Pilot Scale Production of RetroMAD1 Micronized Powder Using Supercritical Fluid Drying (SCFD)

A 10 L high pressure vessel of a configuration conventionally used for Supercritical Fluid Drying (SCFD) was used to dry and produce a micronized form of powdered free-flowing RetroMAD1 under the conditions of 120 bar; 37 C; 300 kg/hr CO₂ flow that gave 88-89% yield in 2 cases and a lower 58% yield in one case due to operator error. The resulting powder was observed to be slightly cubic when viewed under Scanning Electron Microscope (SEM) and about 1 micron in size on the average. This confirms that RetroMAD1 may be efficiently manufactured as a powder for incorporation into tablets, capsules and animal feed pellets whether for terrestrial or aquatic application. The schematics of the process is given in FIG. 27 and a SEM picture as well showing the morphology of the RetroMAD1 crystals is showing in FIG. 28. The parameters used are shown in Table 29.

TABLE 29
Parameters used to manufacture RetroMAD1 crystals

| | Conc | pres- | | CO2 | | | |
|---|---|---|---|---|---|---|---|
| Batch | (mg/ | sure | temp | flow | mass | yield | character |
| (lot no) | ml) | (bar) | (Celcius) | (kg/hr) | (grams) | (%) | (powder) |
| 160113 | 6 | 120 | 37 | 300 | 2.1 | 88 | free |
| B35 | | | | | | | flowing |
| 060213 | 6.2 | 120 | 37 | 300 | 1.7 | 58 | free |
| B37 | | | | | | | flowing |
| 200213 | 6.64 | 120 | 37 | 300 | 2.7 | 89 | free |
| B39 | | | | | | | flowing |

### Bioactivity of RetroMAD1 SCFD Micronized Powder Evaluated Using HSV-2

The bioactivity of the micronized form of powdered free-flowing RetroMAD1 produced using SCFD was tested via antiviral assay against HSV-2. For the antiviral test, RetroMAD1 micronized powder was dissolved in two different solvents: (i) ultra pure water with 5.5 mM NaOH; and (ii) ultra pure water. Ultra pure water was produced using a Sastec ST-WP-UVF machine.

### Cytotoxicity of RetroMAD1 Micronized Powder

Prior to screening RetroMAD1 micronized powder for its antiviral properties, it was subjected to cytotoxicity assay in order to identify the maximal concentration, which could be non-toxic to Vero cells. The cytotoxic activity of the peptides was quantified using MTS-based cell titer 96 non-radioactive cell proliferation assay. Briefly, monolayer cultures of Vero cells were exposed to increasing concentrations of the dissolved RetroMAD1 powder for 24, 48 and 72 h of incubation. After the incubation period, the maximal concentration of the protein that did not exert toxic effect is regarded as the maximal non toxic concentration (MNTD) was determined using MTS assay.

Results as shown in Table 30 indicate that the accepted maximal nontoxic concentrations of RetroMAD1 micronized powder on Vero cells were less than 20 μg/ml. At the chosen MNTD, the peptides did not impair the cell viability with respect to the untreated control group.

TABLE 30
Maximal non-toxic dose of RetroMAD1
micronized powder on Vero cells

MNTD, ug/ml

| Peptide | 24 h | 48 h | 72 h |
|---|---|---|---|
| RetroMAD1 powder (in water + NaOH) | 15 | 15 | 15 |
| RetroMAD1 powder (in water) | 5 | 5 | 5 |

### The Antiviral Activity of RetroMAD1 Micronized Powder Against HSV-2

The antiviral activity of RetroMAD1 micronized powder was evaluated by simultaneous treatment. For simultaneous treatment, the mixture of RetroMAD1 and virus were inoculated onto Vero cells in 24-well culture plates and incubated for 24 and 48 h at 37° C. under 5% CO₂ atmosphere. At the end of the time period the samples were harvested and viral DNA was extracted. The eluted DNA was then subjected to RT-PCR.

The results obtained suggested that RetroMAD1 in powder form exhibited strong inhibitory activity against HSV-2 via simultaneous treatment giving between 85%-100% of inhibition. RetroMAD1 powder was dissolved in ultrapure water with NaOH showed higher percentage of viral reduction compared to the powder dissolved in ultroapure water alone at the MNTD (Table 31 and FIG. 28). SCFD was therefore a viable method of producing RetroMAD1 in a solid dose format good for incorporation into tablets, capsules, medicated chewing gum and aquatic feed pellets.

TABLE 31
Percentage of viral reduction caused by RetroMAD1 micronized
powder in simultaneous determined by PCR.

Time

| RetroMAD1 | 24 h | 48 h |
|---|---|---|
| RetroMAD1 (in water + NaOH) - 10 μg/mL | 98.33 | 93.06 |
| RetroMAD1 (in water + NaOH) - 15 μg/mL | 100.00 | 100.00 |
| RetroMAD1 (in water) - 5 μg/mL | 87.13 | 85.43 |

### Example 20

### Proteomics Study for HSV2 and Dengue Serotype 2 on Vero Cells Using RetroMAD1

A protein profile was obtained from 2D gel electrophoresis and mass spectrometry analysis to study the effect of RetroMAD1 on proteins expression in Herpes Simplex Virus 2 (HSV2) (FIG. 30) and Dengue 2 (DENV2) (FIG. 32) infected cells. 2D gel electrophoresis analysis revealed significantly altered levels of proteins expression, proteins were identified by tandem MS (MS/MS).

Equal amounts of total protein from (i) cells only, (ii) RetroMAD1 treated cells, (iii) HSV2/DENV2-infected cells, and (iv) RetroMAD1 treated-infected cells, were subjected to 2D gel electrophoresis. 250 μg of proteins were rehydrated into 13 cm immobilized pH gradient (IPG) strips (pH 3-11 nonlinear) (GE Healthcare). The first dimension was electrophoresed on the IPGphor III machine (GE Healthcare) at 20° C. with the following settings: step 1 at 500V for 1 hour; step 2 at 500-1000V for 1 hour; step 3 at 1000-8000V for 2.5 hour, and step 4 at 8000V for 0.5 h. After first dimensional separation, the gel was equilibrated as follows; first reduction with 64.8 mM of dithiothreitol-SDS equilibration buffer (50 mM Tris-HCl [pH 8.8], 6 M urea, 30% glycerol, 2% SDS, and 0.002% bromophenol blue) for 15 minutes, followed by alkylation with 135.2 mM of iodoacetamide-SDS equilibration buffer for another 15 minutes. The second dimension electrophoresis was carried out using the SE600 Ruby system (GE Healthcare) at 25° C. in an electrode buffer (25 mM Tris, 192 mM glycine, and 0.1% [wt/vol] SDS) with the following settings: step 1 at 100V/gel for 45 minutes; step 2 at 300V/gel until the run is completed. After electrophoresis, the gels were fixed with destaining solution for 30 minutes, followed by staining with hot Coomasie blue for 10 minutes. The gels were scanned using Ettan DIGE Imager (GE Healthcare). Gel images were analyzed using PDQuest 2-D Analysis Software (Bio-Rad, USA) and only protein spots which showed significant differences (more than 2.0 fold) were selected for mass spectrometry analysis. Identification of proteins was performed by using Mascot sequence matching software [Matrix Science] with Uniprot database.

### Herpes Simplex Virus 2 (HSV2)

The HSV2 replication cycle involves: (1) viral attachment; (2) viral entry; (3) membrane fusion; (4) RNA release; (5) viral protein production; (6) RNA replication; (7) viral assembly; (8) viral transport and maturation and lastly (9) viral release. There are two important HSV viral glycoproteins, namely glycoprotein B (gB) and glycoprotein D (gD) that are essential for facilitating efficient virus entry via the interaction with the host heparan sulphate receptors and associated co-receptors. Glycoprotein B (gB) precursor is transiently associated with calnexin, a membrane-bound chaperone, in the ER that assist in viral entry. Thus, down regulation of calnexin leads to a reduction in virus entry into the cells. Proteins involved in viral RNA release and nuclear transport like Protein disulfide-isomerase (PDI) was upregulated in RetroMAD1 treated cells. PDI has been demonstrated to play a role in redox control at the cell surface. In response to increased extracellular reduction, PDI may help to re-establish redox homeostasis by rearranging and forming disulfide bonds, thereby protecting the cell against this aggression. The viral replication and the increased expression of the viral proteins as well as the introduction of the RetroMAD1 may induce cellular stress to the host cell and trigger the increased expression of the heat shock protein 70 kDa and chaperone proteins including protein disulfide isomerise, superoxide dismutase and peroxiredoxin-6 to respond to the accumulation of unfolded or misfolded viral or host proteins. RetroMAD1 down regulate cofilin1, a key regulator of actin cytoskeleton dynamics that inhibit HSV-induced rearrangements of actin cytoskeleton which is important for infectivity.

Other proteins identified, Glyceraldehyde-3-phosphate dehydrogenase and Triosephosphate isomerase involve in glycolysis pathway were found to be down-regulated. Thus, decrease of energy source needed for variety of cellular processes may lead to the inhibition of replication and amplification of viral DNA and RNA. Proteins involve in viral RNA transcription and translation such as 40S ribosomal protein and Heterogeneous nuclear ribonucleo protein A1 are down regulated and leads to a decrease in viral replication in host cells. Furthermore, nucleolin was found to be down regulated by RetroMAD1. UL12, an alkaline nuclease encoded by herpes simplex virus and has been suggested to be involved in viral DNA maturation and nuclear egress of nucleocapsids. UL12 form a complex with nucleolin, a nucleolus marker, in infected cells. Knockdown of nucleolin in HSV-infected cells reduced capsid accumulation. These results indicate that nucleolin is a cellular factor required for efficient nuclear egress of HSV nucleocapsids in infected cells.

TABLE 32
Fold changes of differential proteins in cells
treated with RetroMAD1, Cell infected by HSV2 and
HSV2 infected cells treated with RetroMAD1.

| | | | | Cells + |
|---|---|---|---|---|
| | | | Cells + | Virus |
| | | Cells + | Virus | (HSV2) + |
| | Cells | RetroMAD1 | (HSV2) | RetroMAD1 |
| Protein Folding | | | | |
| Protein disulfide-isomerase | 0.00 | +1.87 | −5.03 | +2.03 |
| Calnexin | 0.00 | −2.51 | +3.77 | −6.17 |
| Heat shock 70 kDa protein | 0.00 | −1.80 | −9.07 | +1.84 |
| Energy, Transport, | | | | |
| Metabolism | | | | |
| Nucleoside diphosphate | 0.00 | +1.55 | −1.11 | +2.48 |
| kinase | | | | |
| Glyceraldehyde-3- | 0.00 | −1.27 | +2.90 | −1.24 |
| phosphate dehydrogenase | | | | |
| Triosephosphate isomerase | 0.00 | +2.60 | +2.41 | +1.47 |
| Oxidative Proteins | | | | |
| Superoxide dismutase | 0.00 | +1.14 | −3.38 | +3.52 |
| Peroxiredoxin-6 | 0.00 | +1.82 | −1.30 | +1.98 |
| Transcription/Translation | | | | |
| 40S ribosomal protein | 0.00 | +4.7 | +2.78 | −1.73 |
| Heterogeneous nuclear | 0.00 | −1.07 | −2.14 | −1.08 |
| ribonucleo protein A1 | | | | |
| Nucleolin | 0.00 | −1.55 | −10.04 | +17.89 |
| Cytoskeleton | | | | |
| Cofilin-1 | 0.00 | +1.01 | +2.94 | +1.27 |

Symbols “+” indicate upregulation and “−” indicate downregulation.

### Dengue Virus 2 (DENV2)

The DENV replication cycle involves: (1) viral attachment; (2) viral entry; (3) membrane fusion; (4) RNA release; (5) viral protein production; (6) RNA replication; (7) viral assembly; (8) viral transport and maturation and lastly (9) viral release. Differentially expressed proteins identified are proteins involved in DENV reduction. In viral adsorption and entry, Heat shock protein 70 kDa (Hsp70) is an important component of the lipid raft in the DENV entry receptor complex on host cells. The expression of Hsp70 was decreased in the RetroMAD1 treated-DENV infected cells, indicating that viral attachment and entry through this particular receptor complex has been thwarted. The Hsp70 has also been known to interact with LOX1 receptor to initiate cytotoxic T lymphocytes (CTL) response. A reduction of Hsp70 by RetroMAD1 could prevent activation of cross-reactive memory T cells in a secondary infection, a hypothesized mechanism behind dengue immunopathogenesis.

The cytoskeleton is an important part of a cell and one of its main components is the beta-actin which was found to be down-regulated by RetroMAD1 in DENV2 infected cells. Dengue viruses enter cells via receptor-mediated endocytosis, form vesicles, and undergo membrane fusion and their necleocapsid released into the cytoplasm. These movements are mediated by the actin skeleton and in a DENV2 infection study, the actin skeleton has been shown to be an integral part for viral entry, production as well as release. This study shows that RetroMAD1 has shown inhibitory effects before and during DENV2 infection, thus RetroMAD1 may be inhibiting viral entry, viral motility in cells or the synthesis of viral polyproteins. Vimentin is required for DENV2 infection to induce microtubules reorganization for viral replication. Thus, down regulation of vimentin inhibits DENV2 viral replication. Annexins are calcium dependent phospholipid-binding proteins and have been suggested to act as scaffolding proteins at certain membrane domains. Annexin A2 is required for the apical transport of vesicles in polarized cells, specifically vesicles that carry membrane raft-associated proteins. Thus, down regulation of Annexin A2 reduces DENV2 viral replication

During a viral infection, glucose uptake and glycolytic enzyme activity is usually increased for ATP production, a major energy source for cells. The ATPs are necessary in a variety of ATP-dependent cellular processes during viral replication and they are usually catalyzed by viral-encoded enzymes or complexes consisting of viral and host-cell proteins. Enzyme involve in glycolytic pathway identified are glyceraldehyde-3-phosphate dehydrogenase (GAPDH), phosphoglycerate kinase (PGK) and ATP synthase. The reduction of GAPDH, PGK and ATP synthase may therefore lead to the inhibition of replication and amplification of viral RNA. Proteolytic processing of DENV polyprotein is an important step in viral replication and maturation. Following polyprotein processing, viral budding occurs in the endoplasmic reticulum (ER), where they form immature virion and are then transported to the Golgi complex. The human immunoglobulin binding heavy chain protein, a HSP70 member and calreticulin have been found to be involved in DENV protein folding and assembly. A reduction of both Hsp70 and calreticulin in our study after treatment with RetroMAD1 indicates that this RetroMAD1 is able to disrupt production of mature virions. Protein disulfide isomerase (PDI) was down-regulated in DENV2. PDI is an essential component of the endoplasmic reticulum, which is involved in viral translation, replication, and encapsidation. In particular, PDI has been located by in the complex I, the main ribonucleoprotein complex formed with the 3′UTR in dengue virus replication. It is therefore likely that PDI plays a role in viral replication, translation, or encapsidation, and modulation of the expression of this protein would interfere with viral replication.

Other proteins were also detected to be down-regulated by RetroMAD1 in the DENV2 treated Vero cells, including DNA topoisomerase I, 40S ribosomal protein, these proteins' functions have mainly revolved around the DNA replication of host cells. RetroMAD1 may have been suppressing these proteins to inhibit viral replication; however this remains to be addressed. Aldehyde dehydrogenase facilitates the conversion of toxic alcohols to aldehydes and aldo keto reductase is involved in the protection of cells from endogenously formed reactive carbonyl groups. Both of these actions are in favour of cell survival.

TABLE 33
Fold changes of differential proteins in cells
treated with RetroMAD1, Cell infected by DENV2
and DENV2 infected cells treated with RetroMAD1.

| | | | | Cells + |
|---|---|---|---|---|
| | | | Cells + | Virus |
| | | Cells + | Virus | (DENV2) + |
| | Cells | RetroMAD1 | (DENV2) | RetroMAD1 |
| Protein Folding | | | | |
| DNA Topoisomerase 1 | 0.00 | +1.91 | +3.13 | −4.80 |
| Heat shock 70 kDa | 0.00 | −4.49 | +2.11 | −3.93 |
| protein | | | | |
| Calreticulin | 0.00 | +2.21 | −1.50 | +2.01 |
| Protein disulfide | 0.00 | +2.18 | −2.03 | +2.65 |
| isomerase | | | | |
| Endoplasmin | 0.00 | +2.89 | −1.66 | +3.84 |
| Cellular Metabolism and | | | | |
| ATP Synthesis | | | | |
| Phosphoglycerate kinase | 0.00 | +4.36 | −1.61 | −3.19 |
| ATP synthase | 0.00 | −2.05 | +5.30 | −2.19 |
| Calmodulin | 0.00 | −3.09 | +1.20 | −2.06 |
| Aldose reductase | 0.00 | +3.33 | −2.02 | −4.87 |
| Glyceraldehyde-3- | 0.00 | +6.25 | +8.66 | −3.01 |
| phosphate dehydrogenase | | | | |
| Transcription/Translation | | | | |
| Triosephosphate | 0.00 | +2.59 | +3.56 | −1.21 |
| isomerase | | | | |
| nucleolin | 0.00 | +1.03 | −2.22 | +4.62 |
| 40S ribosomal protein | 0.00 | −3.66 | +2.37 | −2.44 |
| Component of | | | | |
| Cytoskeleton/Cytosol | | | | |
| Actin, beta | 0.00 | −3.12 | +4.29 | −3.51 |
| Actin, cytoplasmic 2 | 0.00 | +10.32 | +3.30 | −1.61 |
| non-muscle myosin IIA | 0.00 | −1.89 | +2.41 | −2.44 |
| Vimentin | 0.00 | −3.07 | +11.65 | −8.06 |
| Oxidative Proteins | | | | |
| Aldehyde dehydrogenase | 0.00 | +2.06 | −3.16 | +2.32 |
| Component of | | | | |
| Cytoskeleton/Cytosol | | | | |
| Cofilin-1 | 0.00 | +1.01 | +2.94 | −2.27 |
| Annexin A2 | 0.00 | −3.55 | +2.12 | −2.72 |

Symbols “+” indicate upregulation and “−” indicate downregulation.

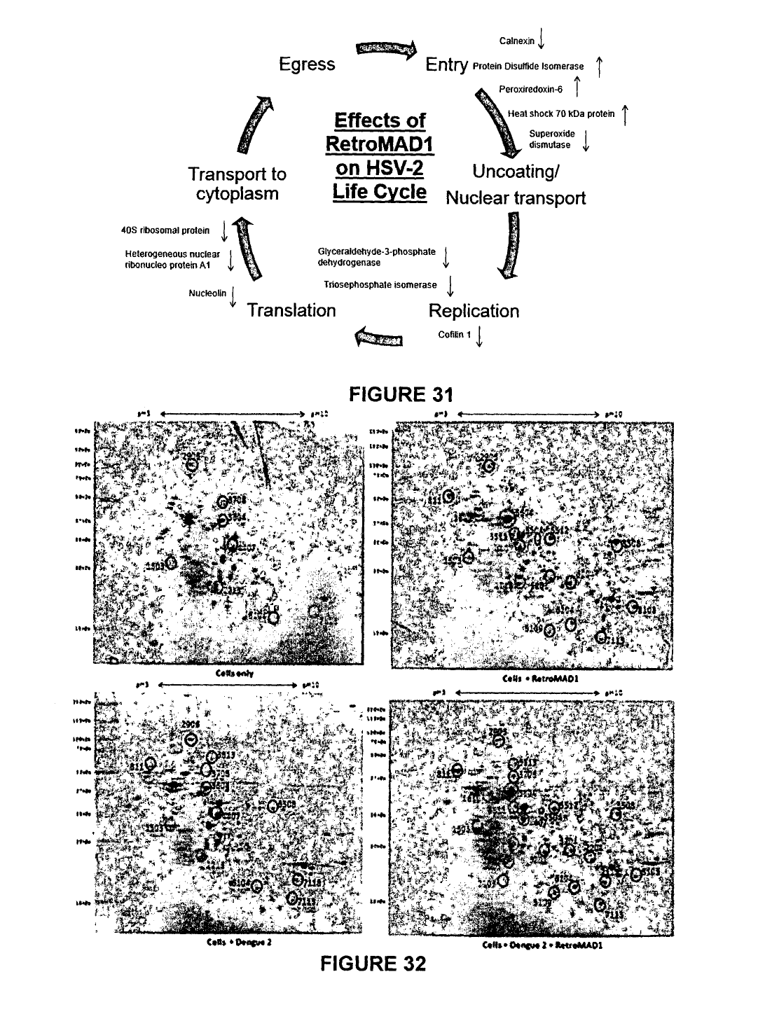

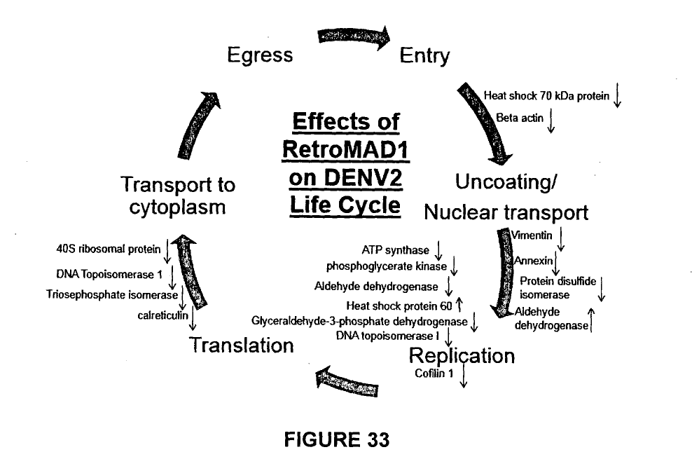

Based on the findings of this study, proteins that are differentially expressed were involved in several biological processes, including viral entry, protein folding, viral transcription and translation regulations, cytoskeletal assembly, and cellular metabolisms. This indicated that antiviral activities of RetroMAD1 could act on various parts of the virus infection pathways, that is via blocking of viral adsorption, replication and also via virucidal effects (Tables 32 and 33 and FIGS. 31 and 33). In conclusion, the inhibitory effect of RetroMAD1 occurred at various stages of viral life cycle and strongly suggests its potential as a broad spectrum antiviral agent.

### Example 21

### Acute Toxicity Testing in ICR Mice for Various Drugs

The acute toxicity study was used to determine a safe dose for RetroMAD1, RetroGAD1 and Tamapal1.

Adult male and female Sprague-Dawley rats (weighing about 200 g±20) were used for the trial. Rats were divided into 3 groups: control, low dose and high dose. Mice were six weeks old. The experimental protocol is provided in Table 34 below.

TABLE 34
Experimental protocol for Example 21

| | RetroMAD1 | RetroGAD1 | Tamapal1 |
|---|---|---|---|
| Groups | Control, Low Dose, High Dose | | |
| Female rats per | 6 | 4 | 4 |
| group | (4 for control) | | |
| Male rats per group | 6 | 4 | 4 |
| Dosing: Control | Distilled water | | |
| Dosing: Low Dose | 20 mg/kg BW | 5 mg/kg BW | 10 mg/kg BW |
| | (4 mg/ml/ | (1 mg/2 ml/ | (2 mg/2 ml/ |
| | 200 g rat) | 200 g rat) | 200 g rat) |
| Dosing: High Dose | 100 mg/kg BW | 15 mg/kg BW | 40 mg/kg BW |
| | (20 mg/4 ml/ | (3 mg/5.1 ml/ | (8 mg/5 ml/ |
| | 200 g rat) | 200 g rat) | 200 g rat) |

*BW = Body Weight

The test animals were fasted overnight (Day 0) prior to dosing on Day 1. The animals were given standard rat pellets and normal saline. Food was withheld for a further 3 to 4 hours after dosing. The animals were observed over a period of 2 weeks for mortality. The animals were fasted on day 14 and sacrificed on day 15 by the use of Ketamine anesthesia. Hematological and serum biochemical parameters were determined following standard methods (Tietz et al., 1983).

The study was approved by the ethics committee for animal experimentation, Faculty of Medicine, university of Malaya, Malaysia. The study was conducted in the Faculty of Medicine, university of Malaya, Malaysia. All animals received human care according to the criteria outlined in the “Guide for the Care and Use of laboratory Animals” prepared by the National Academy of Sciences and published by the National Institute of Health.

RetroMAD1 was fed at much higher doses (4 mg and 20 mg/200 g rat) compared to Tamapal1 (2 mg and 8 mg/200 g rat) while the lowest doses were that of RetroGAD1 (1 mg and 3 mg/200 g rat). The readings obtained for both the male and female fed groups were compared against their respective unfed controls and readings falling outside of the upper and lower limits of the standard deviation of the controls were interpreted as significant to be addressed. All animals survived the trials and no mortalities or abnormal behavior was observed.

TABLE 34
Hematology report for RetroMAD1, RetroGAD1 and Tamapal1 where F is female;
M is male; C is control; LD is low dose; and HD is high dose.

| RBC ×10¹²/ | Hb | PCV | MCV | MCHC | WBC | | B Neut | | S Neut | |
|---|---|---|---|---|---|---|---|---|---|---|
| L | g/L | L/L | fL | g/L | ×10⁹/L | % | ×10⁹/L | % | ×10⁹/L | % |

RetroMAD1

| | | | | | | | | | | | | |
|---|---|---|---|---|---|---|---|---|---|---|---|---|
| F-C | Mean | 7.31 | 148.00 | 0.43 | 59.32 | 341.85 | 8.56 | 1.25 | 0.12 | 10.75 | 0.99 | 80. [symbol] |
| | SD | 0.47 | 10.92 | 0.02 | 3.19 | 13.38 | 3.40 | 0.50 | 0.09 | 3.59 | 0.72 | 4. [symbol] |
| F-LD | Mean | 7.10 | 146.67 | 0.45 | 63.47 | 325.96 | 10.40 | 2.17 | 0.22 | 14.17 | 1.47 | 76. [symbol] |
| | SD | 0.19 | 6.89 | 0.02 | 3.70 | 7.33 | 1.05 | 0.41 | 0.04 | 3.49 | 0.35 | 2. [symbol] |
| F-HD | Mean | 7.43 | 143.50 | 0.44 | 59.47 | 325.00 | 10.42 | 2.50 | 0.27 | 17.17 | 1.84 | 72. [symbol] |
| | SD | 0.33 | 3.89 | 0.01 | 1.58 | 5.67 | 2.33 | 0.55 | 0.11 | 3.49 | 0.71 | 3. [symbol] |
| M-C | Mean | 7.34 | 153.17 | 0.46 | 62.98 | 331.92 | 9.63 | 1.50 | 0.15 | 12.33 | 1.19 | 79. [symbol] |
| | SD | 0.41 | 3.43 | 0.01 | 2.85 | 7.91 | 3.09 | 0.55 | 0.08 | 2.16 | 0.41 | 3. [symbol] |
| M-LD | Mean | 7.39 | 148.50 | 0.48 | 64.64 | 311.45 | 11.16 | 2.17 | 0.24 | 14.83 | 1.59 | 75. [symbol] |
| | SD | 0.33 | 9.27 | 0.01 | 3.18 | 17.08 | 2.69 | 0.41 | 0.08 | 3.31 | 0.23 | 3. [symbol] |
| M-HD | Mean | 7.23 | 146.17 | 0.46 | 63.74 | 317.80 | 9.67 | 2.00 | 0.19 | 15.33 | 1.45 | 74. [symbol] |
| | SD | 0.42 | 7.41 | 0.02 | 2.68 | 5.49 | 3.08 | 0.00 | 0.06 | 4.13 | 0.49 | 3. [symbol] |

RetroGAD1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71. [symbol] |
| | SD | 0.08 | 3.86 | 0.02 | 2.60 | 6.21 | 4.33 | 0.00 | 0.09 | 4.69 | 0.56 | 5. [symbol] |
| F-LD | Mean | 7.46 | 143.75 | 0.46 | 61.72 | 312.74 | 5.00 | 1.50 | 0.07 | 17.75 | 0.85 | 73. [symbol] |
| | SD | 1.06 | 17.95 | 0.06 | 1.03 | 2.58 | 1.43 | 0.58 | 0.03 | 4.86 | 0.14 | 4. [symbol] |
| F-HD | Mean | 7.53 | 142.50 | 0.47 | 62.21 | 305.36 | 5.18 | 1.75 | 0.09 | 18.00 | 0.93 | 72. [symbol] |
| | SD | 0.28 | 4.51 | 0.02 | 3.95 | 19.61 | 0.30 | 0.50 | 0.02 | 3.74 | 0.18 | 4. [symbol] |
| M-C | Mean | 7.54 | 146.25 | 0.48 | 64.19 | 303.31 | 9.98 | 2.00 | 0.20 | 23.00 | 2.28 | 67. [symbol] |
| | SD | 0.64 | 8.18 | 0.03 | 4.21 | 4.32 | 2.35 | 0.82 | 0.08 | 7.26 | 0.81 | 4. [symbol] |
| M-LD | Mean | 7.04 | 137.00 | 0.45 | 63.38 | 307.73 | 6.62 | 2.50 | 0.17 | 22.25 | 1.47 | 67. [symbol] |
| | SD | 0.63 | 10.23 | 0.03 | 2.82 | 5.19 | 0.60 | 0.58 | 0.05 | 3.10 | 0.20 | 2. [symbol] |
| M-HD | Mean | 7.42 | 148.75 | 0.49 | 66.24 | 303.57 | 6.79 | 2.00 | 0.14 | 17.50 | 1.19 | 73. [symbol] |
| | SD | 0.45 | 1.50 | 0.00 | 3.88 | 3.06 | 0.90 | 0.82 | 0.06 | 0.58 | 0.18 | 2. [symbol] |

TAMAPAL1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71. [symbol] |
| | SD | 0.08 | 3.86 | 0.02 | 2.60 | 6.21 | 4.33 | 0.00 | 0.09 | 4.69 | 0.56 | 5. [symbol] |
| F-LD | Mean | 7.23 | 141.00 | 0.46 | 63.02 | 310.48 | 6.74 | 2.00 | 0.15 | 18.50 | 1.28 | 72. [symbol] |
| | SD | 0.38 | 5.03 | 0.02 | 2.80 | 18.49 | 2.10 | 0.82 | 0.10 | 5.32 | 0.58 | 4. [symbol] |
| F-HD | Mean | 7.51 | 141.25 | 0.45 | 59.92 | 316.65 | 5.91 | 2.00 | 0.12 | 18.25 | 1.07 | 72. [symbol] |
| | SD | 0.54 | 11.44 | 0.03 | 6.78 | 34.68 | 0.70 | 0.00 | 0.01 | 0.96 | 0.09 | 2. [symbol] |
| M-C | Mean | 7.54 | 146.25 | 0.48 | 64.19 | 303.31 | 9.98 | 2.00 | 0.20 | 23.00 | 2.28 | 67. [symbol] |
| | SD | 0.64 | 8.18 | 0.03 | 4.21 | 4.32 | 2.35 | 0.82 | 0.08 | 7.26 | 0.81 | 4. [symbol] |
| M-LD | Mean | 7.58 | 151.25 | 0.49 | 64.43 | 310.21 | 9.19 | 2.00 | 0.18 | 25.75 | 2.36 | 66. [symbol] |
| | SD | 0.40 | 7.23 | 0.01 | 2.71 | 10.72 | 0.67 | 0.00 | 0.01 | 6.85 | 0.67 | 7. [symbol] |
| M-HD | Mean | 7.20 | 142.50 | 0.46 | 64.29 | 307.93 | 8.62 | 2.25 | 0.18 | 24.00 | 2.05 | 67. [symbol] |
| | SD | 1.03 | 20.57 | 0.06 | 3.54 | 5.48 | 3.47 | 0.50 | 0.05 | 3.16 | 0.82 | 4. [symbol] |

RetroMAD1

| | | | | | | | | | | | | |
|---|---|---|---|---|---|---|---|---|---|---|---|---|
| F-C | Mean | 7.31 | 148.00 | 0.43 | 59.32 | 341.85 | 8.56 | 1.25 | 0.12 | 10.75 | 0.99 | 80.00 |
| | SD | 0.47 | 10.92 | 0.02 | 3.19 | 13.38 | 3.40 | 0.50 | 0.09 | 3.59 | 0.72 | 4.83 |
| F-LD | Mean | 7.10 | 146.67 | 0.45 | 63.47 | 325.96 | 10.40 | 2.17 | 0.22 | 14.17 | 1.47 | 76.17 |
| | SD | 0.19 | 6.89 | 0.02 | 3.70 | 7.33 | 1.05 | 0.41 | 0.04 | 3.49 | 0.35 | 2.79 |
| F-HD | Mean | 7.43 | 143.50 | 0.44 | 59.47 | 325.00 | 10.42 | 2.50 | 0.27 | 17.17 | 1.84 | 72.33 |
| | SD | 0.33 | 3.89 | 0.01 | 1.58 | 5.67 | 2.33 | 0.55 | 0.11 | 3.49 | 0.71 | 3.72 |
| M-C | Mean | 7.34 | 153.17 | 0.46 | 62.98 | 331.92 | 9.63 | 1.50 | 0.15 | 12.33 | 1.19 | 79.83 |
| | SD | 0.41 | 3.43 | 0.01 | 2.85 | 7.91 | 3.09 | 0.55 | 0.08 | 2.16 | 0.41 | 3.66 |
| M-LD | Mean | 7.39 | 148.50 | 0.48 | 64.64 | 311.45 | 11.16 | 2.17 | 0.24 | 14.83 | 1.59 | 75.83 |
| | SD | 0.33 | 9.27 | 0.01 | 3.18 | 17.08 | 2.69 | 0.41 | 0.08 | 3.31 | 0.23 | 3.06 |
| M-HD | Mean | 7.23 | 146.17 | 0.46 | 63.74 | 317.80 | 9.67 | 2.00 | 0.19 | 15.33 | 1.45 | 74.67 |
| | SD | 0.42 | 7.41 | 0.02 | 2.68 | 5.49 | 3.08 | 0.00 | 0.06 | 4.13 | 0.49 | 3.44 |

RetroGAD1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71.00 |
| | SD | 0.08 | 3.86 | 0.02 | 2.60 | 6.21 | 4.33 | 0.00 | 0.09 | 4.69 | 0.56 | 5.29 |
| F-LD | Mean | 7.46 | 143.75 | 0.46 | 61.72 | 312.74 | 5.00 | 1.50 | 0.07 | 17.75 | 0.85 | 73.75 |
| | SD | 1.06 | 17.95 | 0.06 | 1.03 | 2.58 | 1.43 | 0.58 | 0.03 | 4.86 | 0.14 | 4.72 |
| F-HD | Mean | 7.53 | 142.50 | 0.47 | 62.21 | 305.36 | 5.18 | 1.75 | 0.09 | 18.00 | 0.93 | 72.25 |
| | SD | 0.28 | 4.51 | 0.02 | 3.95 | 19.61 | 0.30 | 0.50 | 0.02 | 3.74 | 0.18 | 4.57 |
| M-C | Mean | 7.54 | 146.25 | 0.48 | 64.19 | 303.31 | 9.98 | 2.00 | 0.20 | 23.00 | 2.28 | 67.00 |
| | SD | 0.64 | 8.18 | 0.03 | 4.21 | 4.32 | 2.35 | 0.82 | 0.08 | 7.26 | 0.81 | 4.69 |
| M-LD | Mean | 7.04 | 137.00 | 0.45 | 63.38 | 307.73 | 6.62 | 2.50 | 0.17 | 22.25 | 1.47 | 67.50 |
| | SD | 0.63 | 10.23 | 0.03 | 2.82 | 5.19 | 0.60 | 0.58 | 0.05 | 3.10 | 0.20 | 2.89 |
| M-HD | Mean | 7.42 | 148.75 | 0.49 | 66.24 | 303.57 | 6.79 | 2.00 | 0.14 | 17.50 | 1.19 | 73.00 |
| | SD | 0.45 | 1.50 | 0.00 | 3.88 | 3.06 | 0.90 | 0.82 | 0.06 | 0.58 | 0.18 | 2.16 |

TAMAPAL1

| | | | | | | | | | | | | |
|---|---|---|---|---|---|---|---|---|---|---|---|---|
| F-C | Mean | 7.56 | 151.25 | 0.49 | 64.87 | 308.81 | 11.71 | 2.00 | 0.23 | 20.00 | 2.22 | 71.00 |
| | SD | 0.08 | 3.86 | 0.02 | 2.60 | 6.21 | 4.33 | 0.00 | 0.09 | 4.69 | 0.56 | 5.29 |
| F-LD | Mean | 7.23 | 141.00 | 0.46 | 63.02 | 310.48 | 6.74 | 2.00 | 0.15 | 18.50 | 1.28 | 72.50 |
| | SD | 0.38 | 5.03 | 0.02 | 2.80 | 18.49 | 2.10 | 0.82 | 0.10 | 5.32 | 0.58 | 4.20 |
| F-HD | Mean | 7.5 | 141.25 | 0.45 | 59.92 | 316.65 | 5.91 | 2.00 | 0.12 | 18.25 | 1.07 | 72.50 |
| | SD | 0.54 | 11.44 | 0.03 | 6.78 | 34.68 | 0.70 | 0.00 | 0.01 | 0.96 | 0.09 | 2.08 |
| M-C | Mean | 7.54 | 146.25 | 0.48 | 64.19 | 303.31 | 9.98 | 2.00 | 0.20 | 23.00 | 2.28 | 67.00 |
| | SD | 0.64 | 8.18 | 0.03 | 4.21 | 4.32 | 2.35 | 0.82 | 0.08 | 7.26 | 0.81 | 4.69 |
| M-LD | Mean | 7.58 | 151.25 | 0.49 | 64.43 | 310.21 | 9.19 | 2.00 | 0.18 | 25.75 | 2.36 | 66.00 |
| | SD | 0.40 | 7.23 | 0.01 | 2.71 | 10.72 | 0.67 | 0.00 | 0.01 | 6.85 | 0.67 | 7.62 |
| M-HD | Mean | 7.20 | 142.50 | 0.46 | 64.29 | 307.93 | 8.62 | 2.25 | 0.18 | 24.00 | 2.05 | 67.00 |
| | SD | 1.03 | 20.57 | 0.06 | 3.54 | 5.48 | 3.47 | 0.50 | 0.05 | 3.16 | 0.82 | 4.24 |

[symbol]

indicates data missing or illegible when filed

RetroMAD1 All data for low and high dose males were unremarkable and comparable against the controls. The females exhibited higher percentages of WBC (White Blood Cells) although the mean numbers were within the standard deviation of the controls. Thrombocyte counts were seen to increase by 40% over the control and there was no significant difference between the low and high doses which will indicate a risk of abnormal blot clots if these data are repeated in primate toxicology trials. Also, it should be noted that these values were not significantly elevated if compared to the other mice from the other control groups for RetroGAD1 and Tamapal1.

In the males, all parameters were within the standard deviation of the mean indicating sex-related metabolic differences may account for the observations in the female group.

### RetroGAD1

In the female population, both low and high doses resulted in a large drop in numbers of WBC, B Neutrophil, S Neutrophil and Lymphocytes. In the male populations, only WBC dropped in the low dose but not in the high dose and S Neutrophils only in the high dose. More parameter deviations were observed in females compared to males.

### Tamapal1

In the female population, only high doses of Tamapal1 consistently caused a drop in WBC, B Neutrophils, S Neutrophils and Lymphocytes in a dose dependent manner. In the males, all parameters were within the standard deviation of the mean indicating sex-related metabolic differences may account for the observations noted only in the female group.

All the drugs tested showed that hematology parameters were very much more affected in female compared to male populations probably due to sex-related metabolic differences. Males were generally unaffected. Nonetheless, as all rats survived and behaved normally, histology data would have to be reviewed in order to get a clearer picture. Nevertheless, it shows that the rats survived 100× and 500× the therapeutic dose given to cats and dogs in multicentre trials for experimentally treating FIV, FeLV and CPV2. The data also shows that RetroGAD1 gave more parameter deviations in females even though the protein concentrations given were the lowest of the three indicating that drug safety from a hematology safety viewpoint was as follows—RetroMAD1>Tamapal1>RetroGAD1.

### REFERENCES

1. Bergmeyer HU, (1980); Clin. Chimica. Acta., 105: 147-154;
2. Brudno M., (2003b); Bioinformatics; 19 Suppl 1:154-162;
3. Cole et. al. (2002); PNAS, V99(4):1813-1818;
4. Cheng, Y.; Prusoff, W. H. Relationship between the inhibition constant (Ki) and the concentration of inhibitor which causes 50 per cent inhibition (150) of an enzymatic reaction. Biochem. Pharmacol. 1973, 22, 3099-3108
5. Karlin and Altschul (Karlin & Altschul (1993) Proc. Natl. Acad. Sci. USA 90: 5873-5877)
6. Sambrook and Green, Molecular Cloning: A Laboratory Manual (Fourth Edition), Cold Springs Harbor Laboratory, New York (2012);
7. Tietz N W, Rinker A D, Shaw L M (1983); J. Clin. Chem. Clin. Biochem., 21:731-748;
8. Wang et. al. (2003); J. Immunol. 170:4708-4716,
9. U.S. Pat. No. 6,652,861
10. Hoskin D. W. and Ramamoorthy A. (2008) Biochemica et Biophysica Acta 1778:357-375;
11. Tang Y Q, Yuan J, Osapay G et al. (October 1999). “A cyclic antimicrobial peptide produced in primate leukocytes by the ligation of two truncated alpha-defensins”. Science 286 (5439): 498-502;
12. Leonova L, Kokryakov V N, Aleshina G et al. (September 2001). “Circular minidefensins and posttranslational generation of molecular diversity”. J. Leukoc. Biol. 70 (3): 461-4.
13. Wang W et at Activity of alpha- and theta-defensins against primary isolates of HIV-1. Journal of Immunology 173(1): 515-520 (2004);
14. U.S. Pat. No. 6,652,861;
15. Tang C et al 2010 Org Biomol Che 8:984-7;
16. Fan, J-M., et al, Mol Biotechnol, 2008, 39, 79-86;
17. Wang et al, Cell, 1999, 99, 433-442.
18. Rohana, Y., Clum, S., Wetzel, M., Krishnamurthy, H. M., Padmanabhan, R. (2000) Purified NS2B/NS3 Serine Protease of Dengue Virus Type 2 Exhibits Cofactor NS2B Dependance for Cleavage of Substrates with Dibasic Amino Acids in Vitro. The Journal of Biological Chemistry 275: 9963-9969.
19. Tietz N W, Rinker A D, Shaw L M (1983). IFCC methods for themeasurement of catalytic concentration of enzymes. Part 5. IFCC method for alkaline phosphatase. J. Clinical. Chem. Clinical. Biochem., 21: 731-748.

1-27. (canceled)

28. A method of improving the oral delivery of at least one peptide to a subject, the method comprising the step of linking the peptide to a MAP30 protein.

29. The method of claim 1, wherein the peptide has antimicrobial and/or anticancer activity.

30-56. (canceled)

57. A method of treating a microbial infection and/or cancer in a subject in need thereof, comprising a step of oral administration before food of a medicament comprising a fusion protein comprising at least one polypeptide B which is a Type 1 Ribosome Inactivating Protein (RIP) or fragment thereof; and
(i) at least one polypeptide A which is an Antimicrobial peptide; and/or
(ii) at least one polypeptide C which is a Cationic Antimicrobial Peptide (CAP) or fragment thereof.

58. The method according to claim 57, wherein the medicament is for administration at least half an hour before food.

59. The method according to claim 57, wherein the medicament is for administration at least an hour before food.

60. The method according to claim 57, wherein the medicament is for administration with a drink and/or medicated chewing gum.

61. The method according to claim 57, wherein the subject is at least one non-aquatic animal.

62. The method according to claim 57, wherein the polypeptide A is a defensin.

63. The method according to claim 62, wherein the defensin is selected from the group consisting of an alpha defensin, beta defensin, gamma defensin, and Big defensin an analogue, or a fragment thereof.

64. The method according to claim 57, wherein the fusion protein comprises the structure A-B-C, A-C-B, C-A-B, C-B-A, B-A-C, B-C-A, A-B-C-C, A-B, B-A, B-C, C-B, C-B-C, or C-C-B-C-C.

65. The method according to claim 57, wherein the fusion protein comprises polypeptides A, B and C.

66. The method according to claim 57, wherein the fusion protein further comprises at least one polypeptide D, which is a synthetic anticancer polypeptide.

67. The method according to claim 66, wherein the polypeptide D is selected from the group consisting of (KLAKLAK)2, SSX2, D-K4R2L9 and p18.

68. The method according to claim 57 further comprising at least one linker peptide between each of the polypeptides A, B, C and/or D.

69. The method according to claim 68, wherein the linker peptide has SEQ ID NO: 3 or 27.

70. The method according to claim 57, wherein polypeptide A is;
(i) a theta defensin selected from the group consisting of Rhesus minidefensin (RTD-1), RTD-2, RTD-3, Retrocyclin-1, Retrocyclin-2, Retrocyclin-3, synthetic retrocyclin congener RC100, RC101, RC102, RC103, RC104, RC105, RC106, RC107, RC108, RC110, RC111, RC112, RC113 and RC114;
(ii) an alpha-defensin selected from the group consisting of human neutrophil protein 1 (HNP-1), HNP-2, HNP-3, HNP-4, Human defensin 5 and Human defensin 6, an analogue, or a fragment thereof; or
(iii) a beta-defensin selected from the group consisting of DEFB 1, DEFB 4A, DEFB 4B, DEFB 103A, DEFB 103B, DEFB 104A, DEFB 104B, DEFB 105A, DEFB 105B, DEFB 106A, DEFB 106B, DEFB 107A, DEFB 107B, DEFB 108B, DEFB108 P1-4, DEFB 109 P1, DEFB 109 P1B, DEFB 109 P2-3, DEFB 110, DEFB 112-119 and DEFB 121-136.

71. (canceled)

72. (canceled)

73. The method according to claim 57, wherein the Type 1 RIP (polypeptide B) is selected from the group consisting of Ebulitins, Nigritins, Amarandins, Amaranthus antiviral/RIP, Amaranthin, Atriplex patens RIP, Beta vulgaris RIP, β-vulgin, Celosia cristata RIP, Chenopodium album RIP, CAP30B, Spinacea oleracea RIP, Quinqueginsin, Asparins, Agrostin, Dianthins, DAPs, Dianthus chinensis′, Lychnin, Petroglaucin, Petrograndin, Saponaria ocymoides RIP, Vacuolas saporin, Saporins, Vaccaria hispanica RIP, Benincasins, Hispin, Byrodins, Colocins, Cucumis figarei RIP, Melonin, C. moschata RIP, Cucurmosin, Moschatins, Pepocin, Gynostemmin, Gynostemma pentaphyllum RIP, Gypsophilin, Lagenin, Luffaculin, Luffangulin, Luffin, MORs, Momordin II, Momorcharins, Momorcochin, Momorcochin-S, Sechiumin, Momorgrosvin, Trichoanguin, Kirilowin, α-trichosanthin, TAP-29, Trichokirin, Trichomislin, Trichosanthin, Karasurin, Trichomaglin, Trichobakin, Crotin, Euserratin Antiviral Protein GAP-31, Gelonin, Hura crepitans RIP, Curcin, Jathropa curcas RIP, Mapalmin, Manutins, α-pisavin, Charibdin, Hyacinthus orientalis RIP, Musarmin, Iris hollandica RIP, Cleroendrum aculeatum RIP, CIPs), Crip-31, Bouganin, Bougainvilla spectbilis RIP, Bougainvillea×buttiana Antiviral protein 1 (BBAP1), Malic enzymes, MAP-S, pokeweed antiviral proteins (PAP), PD-SI, DP-S2, Dodecandrin, PIP, PIP2, Phytolacca octandra anti-viral proteins, Hordeum vulgare RIP's, Hordeum vulgare sub sp. Vulgare Translational inhibitor II, Secale cereale RIP, Tritin, Zea diploperemis RIPs, Malus×domestica RIP, Momordica Anti-HIV Protein, Gelonium multiflorum, Mirabilis expansa 1, phage MU1, betavulgin (Bvg), curcin 2, saporin 6, Maize RIP (B-32), Tobacco RIP (TRIP), Beetins, Mirabilis antiviral protein (MAP), Trichosanthin (TCS), luffins, Momorcharins, Ocymoidin, Bryodin, Pepopsin, 0-trichosanthin, Camphorin, YLP, Insularin, Barley RIP, Tritins, Lamjarin, and Volvariella volvacea RIP.

74. The method according to claim 57, wherein the CAP (polypeptide C) is selected from the group consisting of Cyclotides, Siamycins, NP-06, Gramicidin A, Circulins, Kalatas, Ginkbilobin, Alpha-Basrubin, Lunatusin, Sesquin, Tricyclon A, Cycloviolacins, Polyphemusins, hfl-B5, Protegrins (Pig Cathelicidin), Rat Defensins, Human β-defensins, Temporins, Caerins, Ranatuerins, Reptile Defensin, Piscidins, Lactoferricin B, Rabbit Neutrophils, Rabbit α-Defensin, Retrocyclins, Human α-Defensins, Human β-defensin III (HBD3), Rhesus minidefensin (RTD-1,θ-defensin), rhesus θ-defensins, Human neutrophil peptides, Cecropin As, Melittin, EP5-1, Magainin 2s, hybrid (CE-MA), hepcidin TH1-5, Epinecidin-1, Indolicidin, Cathelicidin-4, LL-37 Cathelicidin, Dermaseptins, Maximins, Brevinins, Ranatuerins, Esculentins, Maculatin 1.3, Maximin H5 and Piscidins, Mundticin KS Enterocin CRL-35, Lunatusin, FK-13 (GI-20 is a derivative), Tachyplesins, Alpha-MSH, Antiviral protein Y3, Palustrin-3AR, Ponericin L2, Spinigerin, Melectin, Clavanin B, Cow cathelicidins, Guinea pig cathelicidin CAP11, Sakacin 5X, Plectasin, Fungal Defensin, GLK-19, lactoferrin (Lf) peptide 2, Alloferon 1, Uperin 3.6, Dahlein 5.6, Ascaphin-8, Human Histatin 5, Guineapig neutrophils, Mytilins, EP5-1, Hexapeptide (synthetic) Corticostatin IV Rabbit Neutrophil 2, Aureins, Latarcin, Plectasin, Cycloviolins, Vary Peptide E, Palicourein, VHL-1, and Buforins.

75. The method according to claim 57, wherein;
(i) the Type 1 RIP is MAP30, the CAP is Dermaseptin 1 and the polypeptide A is Retrocyclin 101;
(ii) the Type 1 RIP is MAP30, the CAP is Alloferon 1 and the polypeptide A is Tachyplesin; or
(iii) the Type 1 RIP is MAP30, the polypeptide D is (KLAKKLAK)2 and the polypeptide A is Gaegurin 5.

76. The method according to claim 75, wherein;
the fusion protein in (i) comprises the amino acid sequence SEQ ID NO: 1;
the fusion protein in (ii) comprises the amino acid sequence SEQ ID NO: 34; and
the fusion protein in (iii) comprises the amino acid sequence SEQ ID NO: 35.

77-80. (canceled)

81. The method according to claim 57, wherein the microbial infection is a bacterium, an archaebacterium, a virus, a bacteriophage, a yeast, a fungus and/or a protist infection or a rogue cell line.

82. The method according to claim 81, wherein the virus is selected from the group consisting of cytomegalovirus (CMV), Epstein-Barr virus (EBV), varicella zoster virus (VZV), HSV-1, HSV-2, HSV-6, BK-virus, influenza viruses, respiratory syncytial virus (RSV); human immunodeficiency virus (HIV), hepatitis A, B or C (HBV), polio viruses, enteroviruses, human coxsackie viruses, rhinoviruses, echoviruses, equine encephalitis viruses, rubella viruses, dengue viruses, encephalitis viruses, yellow fever, coronaviruses, vesicular stomatitis viruses, rabies viruses, ebola viruses, parainfluenza viruses, mumps virus, measles virus, respiratory syncytial virus, Hantaan viruses, bunga viruses, phleboviruses and Nairo viruses, hemorrhagic fever viruses, reoviruses, orbiviurses and rotaviruses, parvoviruses, papilloma viruses, polyoma viruses, adenoviruses, herpes simplex virus (HSV) 1 and HSV-2, varicella zoster virus, variola viruses, vaccinia viruses, pox viruses, African swine fever virus, WSSV, HPV, MBV, IHHNV, YHV, TSV, GAV, LSNV, IMNV, MoV, KHV1, KHV2, KHV3, VNN, pancreatic necrosis virus (IPNV), channel catfish virus (CCV), fish lymphocystis disease virus (FLDV), hematopoietic necrosis virus (IHNV) and viral hemorrhagic septicemia virus (VHSV), AVG, AMAV, swine hepatitis E virus, Circoviruses, Herpesviruses, Porcine cytomegalovirus, pseudorabies virus, Feline Panleukopenia virus (FPV), Feline herpesvirus, Feline calicivirus, Feline Leukemia Virus (FeLV), Feline Immunodeficiency Virus (FIV), Rabies virus, canine parvovirus, canine coronavirus, canine distemper virus, canine influenza, canine hepatitis virus, canine herpesvirus, a virus that causes pseudorabies, and canine minute virus.

83. The method according to claim 57, wherein the cancer is selected from the group consisting of Non-Hodgkin's Lymphoma, brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, pontine glioblastoma, Kaposi's sarcoma, or any other type of solid or liquid cancer.
